SEQUENCE LISTING (65 sequences)

SEQ ID NO: 1 - DNA, 5681 bp, Salmonella enterica
  source 1..5681: mol_type = genomic DNA; serotype = Salmonella enterica Typhi
ttgaaactgaaaacgaatacccatcttttatccgggctacaggcatgtctgttttgtgttaccgctttttttgcccaggcggcgcaaagcagcgccccatggcaggagcctgacgtctcaactgccgccattaacggcacgccgccgctggctgacggcgtgacgatcccggtttatcagggcagcgttcagttgcgatctgatgcagcgaatcccgtcgattattccgcgaaacccagccagttcagcaccagtgatgtcggcagcgcgctgaccttaaccaatccgcgcgatgccgaaggcgatattttcgccgagccgccgctggtctggcagagtgaacggataccctccgtcacgctggtatgggccgacgccgccacgccggagacgccgctgtctccacaatcgtcggcaaacctgacgttctgtgagcaaaatatggcaggccgccatctggtagtctggtcgcaactggatacctcaaccgctatgccgccgctgtggttgcttacccgcaccggcgtgccttataacacggcggtcgaagtgcttgaacaaaagtttgccgtggatatcgcgcccgccgtgggtgacccggtaacccttaccgccgatcatctggatgagtcgcttaacgccgcgaaggtgaaagcgggcgaaagcatcacgctgacggtgaataccaaaggttgtaacggtgagccagcgggcaatatcgcgtttgtgattacccgcggcgatgcgcaaaaccgccagggcgtggtgaacaataccgcgccggtacgcgtcggcaacaccgaactcaccaccactgccaccgaataccacggcacgaccaacgcggaaggggtggcgacggtgaccgtcacccaggctaacgggccgggcgttaaaaccccgctgatggcgcacccgtcgaacgcgcctgcgctgaaagccagcgcggatgtgatttttaccaccctcaccagcccggacagcagcagcgccaacatgtacggccatatggcggacagttccaccgccacggtggacggcgcaagctacacttttgaccgccccaaactggcggcggaaaccgagggcgaagaccgcgtcgccagtatcaataatgaaaactgggcgcagttcacctggggccatgcggataaacactgcgatattctgccggatgcgcgccaactggaagggttaaaaatcgagcgcggcgatttagccaccactctcggctggccggtggggtgacatccggtgacgaagagcactggtcctcctcacagggggcgagcgcaactgaccacatcagcattgatatgcgcagccgcgcccttacgcagatgccggacgccacgcaatcgctggtgagctgtgtggataaagcctcgcctgcggtcacgccgaaattagtgattagcaccgataattttgattcgacggccaacgcggcgaaagtgaaggtgggcgaagagatcaacatgaagatcgccgtcaccgacagcgccaccaataaaccgctgccttatcgctactttaatgtctacctgggcgacgagcaaaatcgccagaatcagaagaacgccgatcttgatgccgcgcatcagtggacggatgaaccggtagttatcgccaatctggacggcggcgacggtcactatcacggcgtcaccgacgccaacggccagttttcgctggcgctgacccaggacaagggcgctggcgtattaaccccggtgcgcgtggtgctgttcgacggcacggaagcgacgcagaatgtgatttttaccgtcgtcaccagcccggacgtgacgcaggcccgtatgtggggccatatgcagggcgtggtggaagcgggcaatatttacaaacgtccgctgctggcggaagaggccgcgcaggataccggttccgaatttgagaataacgagtactgggccacgtttaactcagtgacggcagcgacaaaccagtgcggcgcaggccaggtgccgggacaactgctgctggatacgctgtatgaagcccattccggcaatacgatggaaacgacttacggctggccgacacagaaacatagctatattgcggcggataccgatggttccaccactgcgcacgttaacctggcgaccggcgcggacagcctgttcagcggcgctgaaccgaactatctctcctgctccggtaacgagttggtgaccagcctggacgtgtactttgatggcaacgagtcgttacgtaatgcggtagccaaagtgggcgaaaaaattacgatgaacgtccattccgtcaacgcgctgaacgggttaagcgtgcccaacgccagctttaccgtcaccatgtcgcacggtaaaaatcgcgccaacgccaccacgggctttaccgatcccagcgacggaacgctggtgatgggcggaacgtctttcggctcttccctggcgtcgatgacgtaccagggcatgaccgatgccgcaggcaacgccacgctggtcattgaacaaccgcagggtgtcggcttgctgacgccgctgaccgtactgccggttaactcgctgatcaccacccccgttaaccgcagcgtgaagtttaccgttcccaccagccccgatacgccggatgcgcaaatgtggggccatatgtccgacgccatcacggtgggcgatatgacctttgagcgaccgaaactggcggcggaggttgccgcgacccgcacccaaaccgaggccaacgagagctgggcgcgcgctacccatgccgatgcggtaggcaatacggcggcgggcggctgcgccgctaaccgattgccgcgcgccgatcaactggaggcgctttacgccgctaaccgcgacggggctatcaatagcacgcacggctggccggtgctgattaattactggacctccacctggcaaagcgccacaacgtggaaactgatagcgctttctaacggtagcgaatttccgggcggcgctggcgcatccgattacgtcagttgccttgccagcgataacccgacggcagcctccatcaccattgagccggtgaatacgtcattgtggtatgacgagaacagcgaacacgcggtgaaggtgaaaaagggagatacgctccagcttaaagtgacggtaaaagacgccagcggcaatccgctgccgcaagcgccgttcgtgctcagccgcggcgacggcatcgtctcggcggtagtgattgacggcgattcgctcaacgacaccgccaccaaaatcggcggcatgaccggtgaaaacggcagcaaaatcatcaacgtcacccgcccggatgcccacgggacgaaagtcgcgattacggcggcgctgtacgataacgccagcgcgacggccagcatcgacactatttttactgtggtcaccagccctaatagcgacaaagccaaaatgtggggccatatgccggaaacgaccacggcggccaacggcgtggtgtttaagcgcccgctgttatcggctgaaatcgccagcggctttacccacggcgacaatacggaaaacaacgaagcctggggaatcgtcgattttgaagtggcgaacgacgcctgtggcgcgggatacgtaccgacacttgccgatctgcaatcgctgtacgacgccaggcccggcggcaccatgaatacgcagcagggctggccgctggatggtaaaaactaccagaacagcaccgctgatttgagcaggagtacggaaaaccgctacgttaagtcgattgatctgcgcgatggcggtattagctcgctggcgtggagtgaacaactctatttcgtctgcttgcaaaacgcccatccggcggcgacgcagataacgctcacttcgcctttgtataacgacagcgacggatttgccaaagcgaaagtcggcgagaccatcccggtcatcatcaccacgcgggacgcccagggcaacctggccgccgacacgccggttatctttacgcgcggcgacagcgtcgggcgcgcgaatcaggaagtgaatagcgcgtcggcggcggagatacagataaaccacagcgacgggcgtagcagcggcgttaaatattacaccgccaccggcgctgacggcacgctgacgctgaatatcagccaggacagcggcgcgggctttaaaacgccgctgacggcggtgatagagcacaacggcgtaacgagcgcgctgctaccggtcatttttaccgtcgtcaccagcccggacacgccgaaagccaactactggggccatatggcggaaacgctgaccgacagcagcggcgtggtgtacaagcgcccgttgctggccagcgagttcagcgtcacgcccggcaaatcgctgaccatcgccaacggttactacgacaaaggcgaaacctggggcatgataaccgtcgataaagcctagaacggcgccggcggcggctgcgggcgcaattatctgcccaccgtcagcaacctccagacgctgtacagtacctggcctgataacgccatgcgcagttgtaacggctggccgatgaccagttctggcaacaataacgtcagccgctactggtgggcgggggattacgtgatgtcgtcagacggcgcgcagtcgctctacgcggcggtcaacctgtttaacgacggcaatgatgtcaaaacgacgaccagcaccacgacgtactatatgcagacctgccttacctcgccacgcagcgccgccgccagcctgacgctgacgctggcaggccaggatgcagccaccgggatcgcaaaagcgaaaaagggcgagcagatggccgccaccgtcacggtgaaagacgccgcaggtcagccgatgaaaaacgtgatggtgaaaatcagccggggatcttcgtacaaccgcgtcaacagcgccacatcgtcctcaagcgctgcagacgatattacgctgcgtaatgtgatgccgtccggcccggcgacatacctgctggatacctccgcgaaatatctgtatgcgcaaaccgatgcccagggacaggtgacatttacccttgcgcaggacagtaccgcaggtttaaagaccaccatcagcgccgccacgatggacggcagcaacctgacagacagcaaagacgctatctttaccgttatcaccagcccggatagcgacaaggcctcatactggggccatatgccggaaacctttaccaacagtaagggcgttgagtttgcgcggccgctgctgcgcgccgagctttcttcaacgacggataccacgtccttcctgtcaaataatgagcactggtacacctggaaccgctatccaaacctgtatcaggactccgccagcccgtgcgatcggctgggattgccgacgctggatgatttaaaaacgttgtataacgactatccgaatggtggactaacggccgcctttgggttgcctgtagacgcggggaaatactggggagcaggggattcgaaagtcaacgatacgcactcgacgaataatttccagtatatcaggctaaataacggcgttacgcaggtcacaaatacgaatacctcaacggcgcagctctgtctggcgaagcgcagggtgttggcgatagcgttgacctccagcgccatgaatgccgaaaaatccgccgcgctggcgaaaaaaggggataaaatcccgctgacggtgaccgtcacggacggcgccggcacgccgtag

SEQ ID NO: 2 - DNA, 1188 bp, Salmonella enterica
  source 1..1188: mol_type = genomic DNA; serotype = Salmonella enterica Typhi
atgagtgtgttgagaaaacattacttgaaagggtataccgcgcggcaaattgtacagcgagccatgaaaattattccttactcggtaaacgtcatggatgagcatggcgtcattatcgcgtctggcgaaccttcgcggcttcgccagcgtcacgaaggggccattctggcgctgaaggaaaaccgtattgtagaaattgattccgctaccgccaatcagcttaaaggcgtgcgatccggcattaatcttcccatctcttttcatgaacagcttatcggcgtggtcggcattaccggcgaaccggaggaggttcgcgcgtatgccgagctggttaaaatggcggcggagctggtgatcgagcatatggtgctgatcgaacagtgacaatgggataaacgctatcgcgaagagctgatcaaccaattgattttgcgagaaaattcaacagagtcgttgcgctccatggcggcctatctgggcattgatctggcggttcccagagtggtgctgattattgaactttcccagccggatcgcgaagcgctgcgcaatgtaatggattatttcgagaatcacgcgcgcaaccatttggtgacgtttaccgaatttaatgaattaatcattattaagcctatcacgttaaaagagggaaagtggaatacccgccaggaaatgggcgaattgcagatttttaaatcatgggctgcatcatcgggctttagccgtattttggttgggggctattttgccggcgagacgggattgcaccgatctttgctcacagcgagagccacgcaggcgatggcgaaaagacaaaagctgcgcagccagtacattttttatcatgaccacgcgcttcccgcgctgctaagcgggctgtctgaaagttggcaagtgcaggagttatcgcgtctgtggctgcaactggtgcaacatgacgcgaaaggcgtattgcaacagacgctgcggacttggtttgaacataattgtgacctgacgcaaacggccaaagcattgcatattcatgtgaatacattgcgctatcgcttacagcgctgtgaggatattacgcacataaaaatcaacgagttaaaaagtacgctttggctttatatcggtatggagcttcaggccgaatctgtatcgtccgacaagttaccgctgcctggtcgaatcgaaatttgttga

SEQ ID NO: 3 - DNA, 216 bp, Salmonella enterica
  source 1..216: mol_type = genomic DNA; serotype = Salmonella enterica Typhi
accgataaagtcatccctgaactaaaacagtggcaacagagcccgctggagaaggtttatcccgtcgtctggctggacgctattcattataaaaaccgtgaggatggccgttatcagagcaaggcggtttataccgttctggcactgaatctagaaggcaaaaaagaagttctgggcctatatctgtcggaaagtgaaggtgctaacttttggtta

SEQ ID NO: 4 - DNA, 60 bp, Salmonella enterica
  source 1..60: mol_type = genomic DNA; serotype = Salmonella enterica Typhi
agtgccagaacggtataaaccgccttgctctgataacggccatcctcacggtttttataa

SEQ ID NO: 5 - DNA, 2378 bp, Salmonella enterica
  source 1..2378: mol_type = genomic DNA; serotype = Salmonella enterica Typhi
atgctgaaattctgagcttcattcctgagccttgctctgatgttggccgttccttttgccccgcagccagatcgttcctcaggtgacctcgagtccggcaattaaaaaagcggctaaccacgccgctttttttacgtctgcactcggtaccaaattccagaaaagaggcctcccgaaaggggggccttttttcgttttggtccccgcggtgcgggtgccagggcgtgcccttgggctccccgggcgcgtactccacgaagttcctatactttctagagaataggaacttcggaataggaacttcatgcctttaattaagggcgaccgtcttctatcggtaataacagtccaatctggtgtaacttcggaatcgtccccaattattgaacacccttcggggtgtttttttgtttctggtctaccatctcgttgtgataatagacctgaagtgcctactctggaaaatctttgacagctagctcagtcctaggtataatgctagcagctgtcaccggatgtgctttccggtctgatgagtccgtgaggacgaaacagcctctacaaataattttgtttaaaaagaggagaaatagtccatgcgctgatagtgctagtgtagatcgctactagagccaggcattttatatactggctcgggtaagaactcgcacttcgtggaaacactattatctggtggaagacgtgagcactagtcttggactcctgttgatagatccagtaatgacctcagaactccatctggatttgttcagaacgctcggttgccgccgggcgttttttattggtgagaatccaggggtccccaataattacgatttaaattggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgacttttgtcggctcgacccacgactattgactgctctgagaaagttgattgttacgattagtccggccggccgaagttcctatactttctagagaataggaacttcggaataggaactaccgcggtgcgggtgccagggcgtgccctcgggctccccgggcgcgtactccacgtcagaggagccaaattcaaaaaagcctgctttctagcaggctttttgctttctaatggaagcataaaaaaatggcgccgatgggcgccatttttcactgcggcaagaattacttcagacagagtatcaaaaggcgaaacctccgcaatgcggaggtttctttttaaagactgcagcagcgattgagactcagcgaaccaacaccaataaagtgatctattcgaaccacccggatctggtgcgtccgattgcctcgataaccaaattaatgacagcgatggtcgtgctcgacgcgcgattgccgctggatgaaatactgaaggtcgatatcagccagacgccggaaatgaaaggggtttactcccgcgtgcgcctgaacagcgaaattagccgcaaaaatatgttgctgctggcgttgatgtcatccgaaaaccgtgcggcggcaagcctggcgcactattatcccggcggttataacgcatttattaaagcgatgaatgcgaaggcgaaagcgctgggcatgacgcatacccgctttgttgagccgacggggctgtcgattcataacgtctcgaccgc

SEQ ID NO: 6 - DNA, 1214 bp, Salmonella enterica
  source 1..1214: mol_type = genomic DNA; serotype = Salmonella enterica Typhi
atgtctcgttctatcagaatctgtagctatctgctgctgccgctgatctacctactggtcaatgtcaagattgcccaactgggggaaagctttcccattaccatcgtcacttttttaccgctgttgctgctgttatttgtggaacgcattagcgtaaaaaaattgatgatcgccttaggcatcggcgcggggcttacggcatttaacttcctgttcggccagtcgctgaatgccggtaaatatgtcacgtccacgatgctgtttgtctatattgtggtcattatcgggatggtctggagtatccgattcaaaaccatttccgcgcataaccaccgaaagattttgcgtttttttatctggtggtgggtatagtggtcgcgctcgccgcggttgagatggcgcaaattatccttaccggcggcagcagtatcatggaaggaatttcgaaatatctcatttacagtaatagctacgtactgaacttcataaaatttggcggtaagcgtaccactgcgctttattttgaaccggcatttttcgctttggcactaatctcaatttggctgagcatcaaacagtttggtatcaaaataccgaaaagcgatgctatgattctggcagggataatattatcaggatcattttcaggggtaatgacctttatcctgttttaccttctggagtgggcgttccaatatttgaataaggatgcgataaagaaaaaacttccactggcgctggtatcattaaccctgtttttggttggggtaattattgcatttccttatatcgcgacacgacttggcgatttagggacggaaggattatcttcttattatcgtattgtgggcccgttagtcatggtcggatattccttgacccatattgatggtgtagtcagatttggctcactttatgaatatgtcgcatcattcggaatctttaacggtgcggatgtcgggaaaaccatagacaatggattgtatctgctgattatttatttttcctggttcgcagtgctaatgacgctgtggtatatggggaaagttttaaaaatggcgctaaatgcgtttggcgataatcgcaattttcgggtgcagctctatctttttacgccggtgtcgctgttttttaccggttcaatatttagcccggaatatgcttttttaatcgtctgtccgttcattttgcgcaaggcgttaaaaatttcataa

SEQ ID NO: 7 - DNA, 1869 bp, Salmonella enterica
  source 1..1869: mol_type = genomic DNA; serotype = Salmonella enterica Typhi
atgaaattattaattttaggcaaccacacatgcggcaaccgtggtgatagcgccattatgcgcggtttgcttgatgccatccgtcaacaggcgccagaggcggagatggatgtgatgagccgtttcccggtgagttcctcctggttgcaggggcgaccgattattgccgacccgctgtatcaattaagccagaaacagcaggcggcggcagggcttaacggtcgggtgaaaaagtattacgccgtcgttttcagcataaaattttattgtctaaagtcgcccaggaaggatcgctgcgtaattttgctatcgcgccggaatttgccgaattcgcgcaatttattgcgcagtatgacgccgtcattcaggtgggcggttcaaactatgtcgacttatacccagatcgttcctcaggtgacctcgaggtgaagtgaaaaatggcgcacattgtgcgccattttttttgtctgccgtttaccgcttctctgaaaatcaacgggcaggtcactgacttgcccgtttttttatcccttctccacaccgcggtgcgggtgccagggcgtgcccgtgggctccccgggcgcgtactccacgaagttcctatactttctagagaataggaacttcggaataggaacttcttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaataattaattgagaagttcctatactttctagagaataggaacttcggaataggaactaccggtgcgggtgccagggcgtgccccagggctccccgggcgcgtactccacggaccaaaacgaaaaaaggcccccctttcgggaggcctcttttctggaatttggtaccgagtgcagacgtaaaaaaagcggcgtggttagccgcttttttaattgccggactgcagcagcgattgagactcagcgaacggagcgggcgaaagggttcacgatgatcaaatctgctctcgacagaattcgggaggcgcaatga

SEQ ID NO: 8 - DNA, 362 bp, Salmonella enterica
  source 1..362: mol_type = genomic DNA; serotype = Salmonella enterica Typhi
aaggcgggcaacggcatagacatgagtaacctgtgcatcttcgttctcacccggacggtgtaccagcttctcttccagaccaaactcgaagctgaaatcgtcaccttcacggacaacgcgcgcggaaaggctggcaatctgtccggaacggcgggcaaggtcgatcattccccggtagccaataattaactgaacgtttttttgcctgacttttcgtttttgtttccgaacggcagcagataggcatgaccgagcgcgccgccgggttccagcccaagctgggaacactgaacgatggcgctgacaaaactcatggtgtcacagtcacccagcgccggaacttttcggatttccgttgtggc

SEQ ID NO: 9 - DNA, 1713 bp, Salmonella enterica
  source 1..1713: mol_type = genomic DNA; serotype = Salmonella enterica Typhi
ttactgcgtcgctgcagacggcgtttttgtcggcgttgcgcttaacgatgccggacgagtagacggtacgctatcgcacggtttttcctgcggacaaatcagatcgagcattttcagcgtcacgccgttgctccagccaaagccgtcctgaaggggatattcgccgccgccaccgccggttccggtactgctgacgtcatatttttcgaccagttttttctcgcgatcgtaggtgtgctgcacattggttaaaaagcgccaggtgacttccattgccacgtcatcctgcccataattttgcaatccttcggcagcgacccattgtaacggcgcccagccatttggcgcatcccactgctgtccgcttttaaccgaggtggtagccagcccgccaggctgtagcagatgcgcctgggccgccgctgccactttcgcggcgcgatctttcgcggcggcgtttacatagagcgggaacagcgcggcagcggtgagttggtcacggattttattgttctgcagatcgtagtcggcataccaaccctctttgttattccacagatgcatttcaatggctttttgccgcgcgttggccagcgcgtcatattgcgaggctttggcccgatcgcccgccgcagcgctggcgcgggcgagggttttctccagctgatacagcagagcgttaagatcgacagggacaatcgtggtggtacgaatggtactgagctgctgcggattatccatccagcgggagctgaaatcccagccggaggcggcagcagatcggaggtctcgatagatctccgttgccgggcggctggggttgcttttggcggtagcgatatcttcaacccaggattcagggcggggcgtatcccgatcgtcccagtagcggttgagaacgctgccgtcctccagtttgacgacgcgttggttttgttgccctggctgcaatgtctcaacgccctccatccagtaggcgtactctttttgcagttgcggcaggtattctttcagcgcatcgtcaccttcatgttgcaccagtaattcaaccataaacgcaaagaaaggcggctgtgaacgactcaggtagtaggtacggttgccgttaggaatatacccccaggcgtcaatttcgtaaccaaagttcgctaccatatccgccaccttatcccagtgcccgctttccgccagccccagcatcgtaaagtagctgtcccagtagtaaatctccctgaatcgcccacccggtacgacataggattcaggcaacggcaagagcgagtcccacttttcgacgtttttagttgaacgggtcagcaccggccacaggccatcaatatgttcacgcaatgattgcccggcaggcgggacatatttttcacccgctttcggcagggtgaagttaacatcaacaaaatgacgcaaatcgaagccggactagttccgctgcatacgataatccgcaagaatcatgagcggatcgctattaggtatggcgtcagcaaaggttttctgatcggggaagagttttgcattctggacatcattaaagagcgggccaagcaaaatatccggcggctgcggagtttcagtgtcggaagacgggtctgcggcagtcgccgaaaacgatgcaaacgtcagcagcgtccctgccagcgccagttttatggctttctgtagtagaacagaacggcgaatctctgggggtatcat

SEQ ID NO: 10 - DNA, 1119 bp, Salmonella enterica
  source 1..1119: mol_type = genomic DNA; serotype = Salmonella enterica Typhi
tcatgcctgtttatcctcattgtccggctgttgttcggcttgcgctaactgttgtttgtggcgtttacgttggttgagcgcgctggcgacagcgtgaccgccaacgcccgcagcgaccacgcccagcgcggtcagccccaccgtatcggcggttgaatgggtacccatctggggaatatccaccacgcggctataaaacgagccgcgatcccagaaaccattttctgaacatcccagacatccgtgaccggactggataggaaaggagacgccgtcattccagcgtgtggaggagcaggcgttataggtggttggccctttacagcccatcttgtacaggcagtatcccttgcgggcggcgtcatcatcccagctctcgacaaattcaccggcatcaaaatgggcgcgacggtagcatttatcgtggatacgctgaccatagaacatcagtggacggcccatgcgatcgagttccggcagacgatcaaacgtcaccatataggtgataatggcgctcatgacatccgggattggtggacatccagggactttcacgatcggcttgtcggtgatcactttatcgataggcgttgcctgggtcggattggggcgggcggcctagacgcaaccccaggaggcgcagtttccccaggcgataatagcgctggcgcccgcggcggctttcttcagtttttcaataaacgggcggccgccgctgatacagaacattccttgctctcctaacggcggattgccttccaccgccagaatgtatttcccggcgtagcgagtggtaatatcgtcaaagacttcttcggcctgtgcgccggcggcggccatcagggtgtcgtcataatcgagggaaatcagcgagaggatcacatctttggctagcgggtgcgaggaacggataaaggattcggtacagcaggtgcattccagtccatgaatccagaccaccggaatccgcggtttattctccagcgcccaggcgatctttggcgtcattccggcgcccagtcccagcgatgtggcggcaaggctacagaatttgagaaagctgcgtcgggtcactcccttacgacgcatggcttgataaaaggtctcctcgttattcat

SEQ ID NO: 11 - DNA, 1034 bp, Salmonella enterica
  source 1..1034: mol_type = genomic DNA; serotype = Salmonella enterica Typhi
atgcgggtgattcgtcctgtcgaacatgcggatatcgccgcgctgatgcagcttgcaggcaaaacgggcggcggcttaacctcgctactggcaaatgaagcgacgctggcggcccgcattgaacgcgcgctaaaaacctggtccggcgagctgccaaaaggcgaacaaggatatgtatttgttctcgaggacagtgaaacgggtgaggtcggcgggatctgcgccattgaggtcgcggtcggccttaacgacccttggtataactatcgtgtcggtacgctggttcatgcttccaaagagctgaatgtatataacgcgttgccaacattattcctgagtaatgatcataccggcagcagtgagctttgcacgctgtttctcgatcccgaatggcgtaaagagggtaacggctacttgttgtcaaaatcgcgctttatgtttatggccgcgtttcgcgataaattcaatgagaaagtggtggcggaaatgcgcggtgtcattgacgagcatggttattctcccttctggcaaagtctcggcaaacgcttttttctatggatttcagccgggcggattttttatgcggtaccgggcaaaaagcgtttatcgccgagctgatgcccaaacatcctatttatacccacttcttatctgaagaggcacaggccgtgattggcgaagtccacccgcaaacggcgccagcccgcgtggtgctggaaaaggaaggctttcgctatcgccactatatcgacatcttcgacggtgggccgacgctggagtgtgatattgaccgcgtgcgggctattcgtaaaagccgactggtggaagtcgcggaagggcaacccgcgcttggcgactatcctgcatgtctggtcgccaatgaaaactatcaccactttcgggccgcgctggtgcgtgccgatccgcagacttcacgacttgtacttaccgccgcgcagttggatgcgctgaaatgtcgcgcgggcgaccacgttcggctggtacgcctttgcgctgaggagaaaaccgtatga

SEQ ID NO: 12 - DNA, 971 bp, Salmonella enterica
  source 1..971: mol_type = genomic DNA; serotype = Salmonella enterica Typhi
atgctgtacagggcgcggccacgccccgtacagcgggtaccacagataaaacgagcctcagtgaggaagagaatatgttactcaaaaaacaggtgttatttccagcaaccaaaaaagcgtttggtcttttccgtgacccgttcgccgacgaagccatgcagggttctgatgatgtgttcaccacaccagacattcgctacgtgcgtgaggcgttgtaccagacagcccgtcatggtgggtttatggccgtcatcggtgagtccggtgcgggtaaatccacgctgcgccgtgacctgactgaacgtatcaaccgcgagaatgcgccagtgattgttatcgagccatacatcatcgctatggaagacaacgatgtgaaagggaaaactctgaaggcagcagcgattgccgaagccattatcagtaccatcgcaccactggaaagcatcagacgcagtcaggacgcccgctttcgccagttgcatcgcgtcctgaaagacagcaaccaggcggggttcagccacgttctggtgattgaggaggctcacagtttgcccattccgacactgaaacacctcaaacgcttttttgagctggagtccggtttcaaaaagctgctgtccatcgtgctgattggccagccggaactggcgacaaaactctctgaacgcaacatggaagtccgcgaagtcgttcagcgctgtgaggtggtcgaacttctgcctctggacaatcaccttgaagaatttctgacgttcaaactgcaacgggccggtaaacaactgacggacattatggacgccagcgcagtggatgccatacgtacccgcctgagcaatccgggaagtcatcgtaaaaatatggtcagcctgctgtatccgctggccgtcagtaacctggtaatagccgccatgaatctggccgctgaaatcggggttccacaggtcaacgctgacgttgtcaaaggggtttaa

SEQ ID NO: 13 - DNA, 426 bp, Salmonella enterica
  source 1..426: mol_type = genomic DNA; serotype = Salmonella enterica Typhi
atgaaaaaagtagtggtgttatctgcggtagccgcagccgtgatgatggctggagccgccaacgcagcagaaatctataacaaggatggcaataaactggatttgtatggcaaggtcgacggcctgcattatttttccagtaatcatagtacggatggcgatcaatcttatatccgtatgggtattaaaggcgagactcaaattaccgatcaactaaccggttttggacagtgggagtatcaggtcaacgccaatcgtccggaagatggtgactccagcggttccccgcaaagctggacgcgtctcggttttgctggtttggcatttgccgatatggggtctgttgattatggtcgtaactatggcgtgttatacgacattggttcatggactgacgtactgcctgaatttggtaatattacctga

SEQ ID NO: 14 - DNA, 1372 bp, Salmonella enterica
  source 1..1372: mol_type = genomic DNA; serotype = Salmonella enterica Typhi
ttttaatagccgtacccggcatgacttgcctttgatcttcccgttttgcaactgcttccaggctttttgcgctactgcttgacgtacggcgacgtaaacgtgcatgggatgcacgttaattttgccaatatccgccccgtccaatccaatatcgccggtcagcgcgcctaaaatatctcccggacgcattttcgcttttttgccgccgtcaatgcatagggtagccatctctgcggccagagggagtaacggctgccgggcgggcgcattcagccagttcagcttgagttgcagcatttctgaaagaatattcgcccgctgcgcctcttccggcgcacagaaactgatcgccaggccgctgcttcccgcgcgcgccgtacggccaatacgatggacatgcacctccgggtcccaggccagctcatagttaaccaccagttcgagcgatttaatgtctaaccctcgcgcggcaacgtcggtggcaaccagaatgcgcgcgctgccatttgcaaaacgcaccaacgtctggtcgcggtcgcgttgttccagatcgccgtggagcgccaacgcgctttgtcctaccgcattaagcgcatcacaaacggcctgacaatcttttttggtattgcaaaacaccacgcaggacgctggctgatgctggctaagcaacgtttgtagcagcgaaattttttcatgcgccgacgtttcgaagaactgctgttcgatagccggtagcgcatctaccgtatcgatttcaatacgtattggctgctgctgtacacgaccgctaatcgccgcaatggcctcaggccaggttgctgaaaacaacaacgtctggcgcgtcgcaggcgcaaagcggatcacctcatcaatggcgtcactgaatcccatgtccagcattcggtctgcttcatccattaccagaatgtgcagcgcatccagcgatacggtttctttttgtaaatgatccagcaggcgccccggcgtcgcgacaatgatatgcggagcgtgctgaagcgagtcgcgctgtgcgccaaagggctgcccgccacacaaggtcagaattttggtatttggcagaaaacgggccaggcgacgtaactccccggctacctgatccgccagctcccgcgtcgggcacagcactaatgcctgtgtctggaacagagtgacatcaattcgatgcaagagcccaagaccaaacgccgccgttttaccgctaccggtcctggccttgcacacgcacatcattacccgccagaatgacgggtaatgctgcggcctggacaggcgtcatctcaagatagcccagctcagtaaggttattgagctgggcggcgggcaaaacattcagggttgaaaaagcggtcac

SEQ ID NO: 15 - DNA, 345 bp, Salmonella enterica
  source 1..345: mol_type = genomic DNA; serotype = Salmonella enterica Typhi
ttcttttacggttaacgcttctgtttttttcttcttctccagcaccatctggcgacgaggatcgagtccctcagttaaccatgatctaaagtactgacggcgttccctggcatgagatagtgatatggttggataatcgccaatcgtcagttgagcagcgttcccgttccatctgtatcggtaaaagaatgttatactgccggatgtagagagcctgacattgagaccatgagcgtctgagatgaccttgatttggtctctttttttgccaggagcctttcttaattttgtatcggcaagcaatgtgtacactccataagaggatatacacatctgtgtatacat

SEQ ID NO: 16 - DNA, 459 bp, Salmonella enterica
  source 1..459: mol_type = genomic DNA; serotype = Salmonella enterica Typhi
gtgtctcagccttgtccctgtggtagcgctgacgagtatagcctatgttgtggtcgtattgtgtccggagaacgagtagcacccgatccgtcacatctcatgcgctctcgttactgcgcttttgtgatgaaagacgcagattacttaattaagagctggcatccaacttgcaatgcggccgcgtttcgtgatgatatcatcgccggatttgccaataccaggtggctcggcctgactatttttgaacatacctagtcagaagcagaaaatacagggtatgttagttttatcgcgcgtttttccgaacaagggaaaaacggggcgattatcgaacgttctcgttttatcaaagaaaacggtcagtggtattatattgacggtacccgcccgcagttgggtcgaaatgatccctgcccgtgcggttcaggcaaaaaatttaaaaagtgctgcggccagtga

SEQ ID NO: 17 - DNA, 3625 bp, Salmonella enterica
  source 1..3625: mol_type = genomic DNA; serotype = Salmonella enterica Typhi
atgtctttcattcaataccgccgggacaaacaccttccgtccaccgccgcaccgtcgctgttagctatgggtatggcgatggcatttatgcctgccgccttcgcggctgaggataccgttattgtcgaaggcgcgacgaccgctgacgccataaaccgtgaagaacaggattacagcgtgaaaacgaccgcagcgggcaccaaaatgccgatgactcagcgcgatatcccgcagtcggtcagtattgtcagccaacagcgtatggaagaccagcagttgcaaaccctgggcgaggtgatgaccaatacgctggggatcagcggaagccaggctgactccgatcgcatcagctattactcgcgcgggtttgaaattgacaactatccagatcgttcctcaggtgacctcgagagttaaccaaaaaggggggattttatctcccctttaatttttcctcgcagatagcaaaaaagcgcctttagggcgcttttttacattggtggcggtgcgggtgccagggcgtgcccctgggctccccgggcgcgtactccacgaagttcctatactttctagagaataggaacttcggaataggaacttctagtagcccgcctaatgagcgggcttttttttaattcccctatttgtttatttttctaaatacattcaaatatgtatccgctcatgagacaataaccctgataaatgcttcaataatattgaaaaaggaagagtatgagcattcagcattttcgtgtggcgctgattccgttttttgcggcgttttgcctgccggtgtttgcgcatccggaaaccctggtgaaagtgaaagatgcggaagatcaactgggtgcgcgcgtgggctatattgaactggatctgaacagcggcaaaattctggaatcttttcgtccggaagaacgttttccgatgatgagcacctttaaagtgctgctgtgcggtgcggttctgagccgtgtggatgcgggccaggaacaactgggccgtcgtattcattatagccagaacgatctggtggaatatagcccggtgaccgaaaaacatctgaccgatggcatgaccgtgcgtgaactgtgcagcgcggcgattaccatgagcgataacaccgcggcgaacctgctgctgacgaccattggcggtccgaaagaactgaccgcgtttctgcataacatgggcgatcatgtgacccgtctggatcgttgggaaccggaactgaacgaagcgattccgaacgatgaacgtgataccaccatgccggcagcaatggcgaccaccctgcgtaaactgctgacgggtgagctgctgaccctggcaagccgccagcaactgattgattggatggaagcggataaagtggcgggtccgctgctgcgtagcgcgctgccggctggctggtttattgcggataaaagcggtgcgggcgaacgtggcagccgtggcattattgcggcgctgggcccggatggtaaaccgagccgtattgtggtgatttataccaccggcagccaggcgacgatggatgaacgtaaccgtcagattgcggaaattggcgcgagcctgattaaacattggtaaagaagttcctatactttctagagaataggaacttcggaataggaactaccggtgcgggtgccagggcgtgcccccgggctccccgggcgcgtactccacggaccaaaacgaaaaaacaccctttcgggtgtcttttctggaatttggtaccgagcctttggtcgaaaaaaaaagcccgcactgtcaggtgcgggcttttttctgtgtttccctgcagcagcgattgagactcagcgaacggttgacggtattccaacgtattttgagtcccgctggaatctgggcgatgcgctaacggataccgcgctgtatgagcgcgtggaggtggttcgcggcgcgaatgggctgatgaccggaaccggcaatccttctgcctcaattaatatgatccgtaaacacgccaccagtcgggagttcaaaggtaacgtctccacagaatacggtagctggaataagcagcgttacgtcatggatctgcaaagtccgctcaccgcagacggtaatgtccgtgggcgcatcgtggcgggttatcagaataatgattcctggctggaccgctataacagtgagaaagcgttcttttccggaattatcgatgccgatctgggtgcaactaccaacctttcagccggttatgaatatcaaaaaattgatgtcaacagcccgacctggggcggtctgccgcgctggaataccgatggcagcaaaaatagctatgaccgcgcgcgtagtaccgccccggactgggcttataacaataaagagataaataaattcttcgtcacgcttaagcagcgttttgccgaaagctggcaggcgaccctgaatgcgacccatactgagggtcaaattcgacagtaaaatgatgtatatcgatgctttagtggataaagagacaggtacgctggtgagtccttatggcgcaagctaccccgtggtcggcggtactggctggaacagcggcaagcgcaaagtagatgccatagatctctttgctgatggcgcttatgagctgtttggccgtcagcacaatatgatgtttggcggcagttatagtaaacagaataaccgctattttagcgcctgggcgaacgtctttccggacgacattggcaacttcggcgcctttaacggtaatttcccccaaacccactgggcgccacaaaatctggcgcaggacgataccacgcatatgaaatcgctgtatgccgctacgtgcatttcgctggccgacccgctgcatctgattcttggcgctcgttataccaactggcgtgttgatacactgacctacagcatggagaaaaaccataccacgccgtatgccgggctgatttatgatatcaacgacaactggtccgcttatgccagttatacctctatctttcagccgcagaataaacgcgacaaagcgggtcaatatctggctccgattaccggtaataactatgaagcgggcctgaagtccgactggatgaacagccgcctcaccactacgttgtccgtgttccgcattgagcaggataatgtcgctcaagcaaccactattcccatccctggcagtaatggggaatttgcctggaagtctactgacggaacggtgagcaaaggcgtcgaatttgaagtgaacggcgcaattaccgataactggcagatgactttcggggctacccggtatgttgccgaggacaatgaaggtaacgccgtgaaccctaatctcccgcgcaccagcgtgaagttattcacccgctatcgtctgccggcgataccggagctgacggtcggcggcggcgtgaactggcagaatcgggtttacaaagataccacgacgccatacggcaccttccgcgccgaacagggcagctatgcgctggtcgatctgtttacccgctatcaggtgacgaaaaatttctccgtacagggaaacatcaacaacctgttcgataaaacctacgacaccaatattgatagttccattgtttacggcgcgccgcgtaacgtcagcctcaccgccaactatcagttctga

SEQ ID NO: 18 - DNA, 573 bp, Salmonella enterica
  source 1..573: mol_type = genomic DNA; serotype = Salmonella enterica Typhi
atgcaatttaaaaatactccacaacgttatggcgtagtttccgccgccctccactggctgaccgccctagtggtctatggcatgtttgcgttgggtttatggatggtcacgctcagttattacgacggctggtatcactaggcgccggaaatacataaaagtattggcatgttactgatgatggcgctgatcgtgcgtattatctggcggctttattctccgccgcccgttgcgttgaccagctattcccgtttaacgcgcattggcgccgccgcgggtcatatccttctgtatctcctgctctttgcgataatcattagcggctacctgatttccaccgccgacggtaaaccgattagcgtctttggctggtttgagattccggccacgcttacggacgcgggcgcgcaggctgacatcgccggaacactgcatctgtggtttgcctggtcgctggtcattatctcgctctcgcatggggttatggcgctaaaacaccatttcatcgataaagacgacacactgaaacgtatgacaggaatgtcgtcatctgactatggagctcaaaaatga

SEQ ID NO: 19 - DNA, 1466 bp, Salmonella enterica
  source 1..1466: mol_type = genomic DNA; serotype = Salmonella enterica Typhi
tcaaactccccattttgggataggatggtcgcccattgagatgcagacgtttttcatttccgcaaacacttcgaagctatattcgccgccttcgcgcccggtaccggaggctttcacgccgccgaacggctggcgcaggtcgcggacgttctgggtgttgacgaagaccatgccggcttcaatcccacgcgccaggcgcaacactttgctcacgtcctgggtccagatataagaggccagaccgtattccacatcgttcgccaaacgtaacccttccgcttcgtctttgaatggcagcaggcaggcgaccggcccaaagatctcttcctgcgcaacgcgcatacggttgtcgacatcggccagcacggttgggcgcaggaagttaccgcctttcagatgcgcaggcaggtcagtgggtttttccgcaccgcccgccagcagcgttgccccctcttcaatgccgaggcggatataaccggagactttctcccagtgctgttggctaatcagcgcgccgacctgggtgttcgggtcggtcggatcgccgacacgcaggcgattcgcgcgttcggcaaagcgcttcacgaactcagggtaaatgctctgctggataaagatgcgcgacccagcggtgcagcgttcgccgttgatcgagaagatggtgaacagcgcggcgtccagcgcgcgctcaatgtcggcgtcttcaaaaatcagcaccggcgatttgccgcccagctccatcgagtattttttcagcccggcatttttcatgatattgcgaccggtggcggtaccgccggtaaacgacaccgcacgcacgtcatggtggcgtaccagcgcatcgcccgccgtcgcgccgtagccctgcaccacgttcagcacgcctgccggaattcctgcctccagtgccagctcgcccagcctgtcggcagtcagcggcgacagctcggacattttgagcaccgcggtgttacccagcgccaggcacggcgcaactttccaagtcgcggtcataaacggcacgttccacggcgacaccagcgcgcagacgccgacgggctgcaccagcgtataattgagcattttatcgtcaaccggataggtcttgccgttcatctgctggcacacttcggcgaagaattcgaagttatgcgaggcgcgcgggatcagcacgttttagtctggtgaataggcaggccggtgtcggcggtttccatcgccgcgatttccggcacatgctggtcaatcaggtcgccaaggcggcgcatcaggcgcgcgcgctctttcatcggcaggttggcccatttcgggaacgcctcttttgccgccgcgacagcctggttcacttctgcttcaccgccggaggctacttccgccagcacatcaccggtcgccgggttagtggtctggaagtagtcgttacctgcaacgtttttgccgttaatccaatgatttattttcttcat

SEQ ID NO: 20 - DNA, 1491 bp, Salmonella enterica
  source 1..1491: mol_type = genomic DNA; serotype = Salmonella enterica Typhi
ttaacgtacctgactgggcagacacccataatgttggcggaatctggccgtaaatcgcgaccccgacaggtagccgcaccgtagcgctatttcgccgatcggcagcgtcgtcgattgcagttgagaaagcgcgcaggacatgcgtacctcttcaataatttgccgataactctgcgattcgcgctgcaaacgccgacgcagcgtcgatgttcccatagcaaggcggcgggctatctcctgggctgtccatagcttagcaggtgagagcataatcagttgccgcacctgctccgtgagggtgtaacgccgttcaataagcagcggtcccgccgcgccatcatgtaaaagcgctaacagtaaccccatcgcctgatgttcctgtagcccaacgggcaacccctggcgcacagcatccaacacgttctcccacataaatgtcaggctacggctcatcggcgtacacagcgatgtcaggtttgccggtggataatcctgaacatacatcgttttaaagcgagcaataatctccggtgaaagtaacaacaggtcggagcgaaaaccgttctgcgcaggctgattaataaatatcctccggcatagccggaggtttttctgatgcgcctgtaaggctctcttaccagccgcgccctaacaggcgcatacgatctgacatttgcatcaaacttcgttacttacggcccgtaaacaggctgcccggatacgggatcgataattgctcacccattttatcctcttcaagctggtgctttatgtagtcctgtatcttcgccgtattcttacccaccgtatcgacatagtaccctctgcaccagaactccctgttcctgtatttgaattttagatccccaaactgctcgtaaagcatcagactacttttacccttcagatatcccatgaaactcgacacactcatcttcggcgggatctccagaagcatgtgaatatgatctgcacaacattccgcttccagaattcgtacgtttttccattcacacaattttcttaatatgctgcctactgccctacgcttctctccatagaacgcttgtcttcggtatttgggcgcgaaaactatgtgatatttacagttccatcgggtgtgcgctaagctcttttcgtcccccattgggacccccttttgatttcttgttgaacttttgcagttgccagaccgcaagatgttttaacaaatcaaaaggggttttaataactggcttaaagctgaaagctttccggaacccccagcctagctgggggttttccatagacaataatctccagcggcgtgttcgccggaataataattaactcgccaggcccggcgacaaggcggctatcatcctgaatgatgactttactgccctgcgtaatatggcagatcgcggcggaaaaaagcgtaacacgatgcaaacggtgcagatgactggaacgcacctctgccgtcgtaagatcctgatgtcgaatatgcat

SEQ ID NO: 21 - DNA, 788 bp, Salmonella enterica
  source 1..788: mol_type = genomic DNA; serotype = Salmonella enterica Typhi
tcataataattgcggtggcgcaaatcgtcgttgctgcaaaagaggaagttgctcccgcacctggtctattcgttctctgaatatttccgcgacgatcagcgccggacgatcggctgccgcggctatcgttacccccaacggtcgataatacgactttgaccaatattacgattaccacactccccggccgcaatcatatagcaggtggtatccagcgctctcgccgccagcagcgtcgaccactgctgctctttcaacgggccgcgaacccagcccgtcggcagcgccaatacgtcagccccctgtaaagccagcgctaacgccatatccggaaagcgcaggtcataacaggtcataagacccaccttaaccccctccacgtccagcacaggcgcgataacggttccggcatcaatactttgggattcttgcatcgaaaacgcatcatagagatgcagcttcgcgtaacgcgcgacaatgtgacccgcccgtagcgccaccagcatattaaccgcccgtccaggcgttgacgggacaaggatagtgaatatcgtcgtcatattattatgagcgctttcttccagaagccgcgtcataaacgcgccatccagcggctgcgcggcgcgaatcggcaggtcaaggtcgatgtcatctcgcgctaatatcccctcaggcaacaccagaagcgatacgccgcggcccgccgcctgagccattaacgaaacgcaaacttgcacattctcttcccatacagaactcaccacaaactgcccggctgcaacgaacat

SEQ ID NO: 22 - DNA, 1137 bp, Salmonella enterica
  source 1..1137: mol_type = genomic DNA; serotype = Salmonella enterica Typhi
tcagactaaccgttcatctatcgccagcgcgttttccattttacgcgagaccatcgcgtgacgcagtaatacgccgccgcaggccatcataccgccaatcaacgccgccaggataataatgatcgctttacccgggccatcttttttcactggcagagacggcgacagttgatatttaaacggggtaaacttcacgtcgctcacattcattgccgccagttgttcaacatagtattgacggttacgcaaatcaccgtcgatctcggccacgtccgttacccctttttcaatttccagtttgcgggaaataccatccgcgccgagggaaatagaaaaatccggatcatcttttaccgcctgaccattactgtaaaccggtctcttaatgccagcggcgttggcgatttccagcgaataatgaagacgttgaatattggcatcaagctgatttttgagacgcacccgatccatcgccagcttttcctgctcgtagcgggttttgatttccagctggttacgaatattttccagcgtctctttcacgacgatatcggagatgtactgaatatagccagccaacactttttgcgcttcttcccgcgtcggcgcggtaaaactcagcgtccacgacgtgaataacgacgtttcatttttcttgccggcattgctgtccaccgctttcattttttcgctcagcacgacaatcgcccggtgaagatcctgctcgtctatttgcgcgccttttaattgatccatgacatacggagaagaacgaagatattcttccagcagcgagggcgagctaaactttttaataaacagattaaatacgctggcccgatcaacgcttacctccatatccaacacgcgcagcgcggtcaacgttctctccagcccctgctactgtaccgactccgccggtgtgacaatcgcctggctggtccatttttgcggcagcagaaaggacagaagcaaccccacgcacgcaaaggcgaaaacggtagcaagaatacgacgtttcgcctgccataacacctctataaggctaaacaaatcgatttcatgactgttggcgggcggcagtgagtaacctgcaaatgactgatttttctcttgttttacattaagagatggcat

SEQ ID NO: 23 - DNA, 1455 bp, Salmonella enterica
  source 1..1455: mol_type = genomic DNA; serotype = Salmonella enterica Typhi
ttaacctgcgactttttgctctcctgtattttcaaaacccgtgcgtttcttcagtagcgcataagcaacgaacgcaactattacgcctacaaaccaggacacgcgggataaaggctccataaatggtataaacttgccgcctaatgacagaatgactgcgaccagggtgactgaaaatgcagtcaggttaaatccattatcataatatttgtaatcaccgctggcggtgtatagctcatcaagattaatttttccacgcattaccacaaaataatgagctaacattacgccgattaccgggccaagcataccgccgataatatcgaggaacagataaatactgtcctgattctccattaatttccatggacaaatcagtagactgataatactggcaatcattacgccatttttatagttaagctttgtcggggtaagcgccgcaatttgataccccgcaggtataatattaccggtggcgttggttgaaattgtcgtcatcagaatcaccagcactgcaaagaatgaagcaaacaggctgtcccagcgctgcacaatatccagcacgttccaggtatccataccataatgaatactggctccggcaataatgcacacgctggctacggcgaataatatatacgctacgataagccccaatgtttgtcccaatgcctgagcgcgaaatgaatgcgcgttttgcgtgaaatcggacgcgctcaccgctggcgcagcccagacggcgactacggcgttaatcaccactaggaacagaaagccgctgtgctctgctttttgcacgcctgaaggcagatagtccaaaatcgggccaatgccgaccagcgatattgcccaaatagccatgccgccaaagacaatataaatacatggattgaggatagcggtaaatttatttaatactttaccaccgccaaaaccgatgccaacgttaatgatccaaaaaattagaaaagtaattagccctggcagtgaaaaacccagcagcttgaaatctccgcctaatgtctaaaatcctggccagatcttcccaattaaaataagaaatgccagcgatcccgcgtaacactgtaagccgaaccacataattgccgcgattccccctcgtaataatccagggaatagcgcgccgcggacgccgtaagaacctcgcaatatcatagcaaaaggaacgccatatttgctgcctgccgcgccattcattaccatcgccgccgcaataaataatgcgctgataataatggccaacatgatattaaatgttgatagccccagtataaaaaaaccgccgaccataacgtaatttggcacgttatgtacagatcccatccataaggtgaaatagttaaatgctttccagtttcgttgggtttctgttttaggtaataagtcttcgctataaccgcgttgctgatatagctctctttgatgttccat

SEQ ID NO: 24 - DNA, 1652 bp, Salmonella enterica
  source 1..1652: mol_type = genomic DNA; serotype = Salmonella enterica Typhi
ctacagccagttcacctcgccatttggcgtaaacgccgccgcatccaacggtgaattctgctgaataaactctttcagcacttccgcgtcaataaacccggtattcacgtagcccggtttgttatcgatgcgcggataaccatcgccgcccgtggcgttgaaactcagcgtcgccatgcggtaggttttggccggatcaacaggctcgcctttgattttcagatcggtgagcttgccctctttggcgacaaagctcacattggcgaactgtggataggcgccggagtccggtttcatctgcgctacggcggtgagataatcaaccacctctttgccgctcatatcggcatacaccacaatgttgccgaacggctgtaccttgagcacgcttttataggtaatatctcccgcctcaatcgagtcgcgaataccgccgccgctcatcacgccaaaatcggcaccggtgcgcgcgatctgcgcagccagaatcacccgtcccatattggtctggacaaatctgaccttactgcgatcgccttcaagcaggccattcacgctaccaattttcacctccagttgcgctttacctttattctggaacggcgttaataacgagagcatttgcggattttcggcgatttccggcgtgtaaagtacacgctcgcttttcccgttatcccaggtcacttttttcttgagatttaccggaataagctggtagttaaccattttcatctcgccgttacggaattcgaaatccgcgtggcccacatatttaccccactcatgcgcctgcacgctccagatgccattttgcttatccggcgcgcagggcgttcccgttacgtaattcacctgtttttattttccgacgccatgcataccgggtcttgtgagtgaccgcccacaatcatcgccagcgaaccggcaggcagactacgcgccatctcaacgtcgcccggcgcattcgaaccgtgatcgccgttgtcataatgtcccatatgcgtggtcgcgataatcacgtccggtttttcattcatattaagttcctgaatcaccacctttgcttcttcagcaggtttacgaaactcaatatcggtgaaatattccgggttgcctattttcgccgtgtcatcggtggttaagccgattaccgcgatttttatatcctggcgtgtaaaaatagcccacggcttaaacagacgctcgccggtacttttttgataaatattggcggaaagaaagggaaacttcgcccacttttcctgctggcgcaatacggtgagcggattatcaaattcatgattaccgacggccatagcgtcgtagccaatcagattcatcccgcggaaatcgggctccacatcctggagatcggattccggcactccggtattaatgtcgccgccggataacaacaggacgcttcccccctcttgcgccacctctttacggatactgtccaccagcgttttttgcgccgccagaccatattcgccatattcgctgcgccagaagtgaccgtggtgatcgttggtatgcaggatagtaattttataggttttatctttttcgtaagcctgtgcaggctgagtcgtcagcgcgaacgccgccagtaacgccagcgccacaccccgtttcaaaaatttcat

SEQ ID NO: 25 - DNA, 516 bp, Salmonella enterica
  source 1..516: mol_type = genomic DNA; serotype = Salmonella enterica Typhi
atgctgttacagacgctggaagagaagctggcgacgctacgccaacgctgcgcgccgctggcgtaacatgcgacgttaagcgcgcgctttgaccgacaccttttccgtacccgcagtactctgttgcagggatatctggaagaggcagacgccaatctcgccgcgttgcgtcaggcggtaaaacatgagcaactgccgcaagtcgcctggctggcggaacatctggcttcgcaactggaggcgatatcgcgtgaaaccgccgcctggtcgctgcgccagtgggatgccgccgcaccagggcttggccgttggcagcgcagacgaatacagcatcaggagtttgaatgtcgactgctggcgatgacacaggagcgcaaaattcgtctggcgcaggcgaccggccttgtcgaacaacaaacgctgcaaaaggaagtcgagatctatgaaggacggctggcgcgctgccgacatgcgctggagaaaatagaaaacgtactggcgcgtttaccccgttaa

SEQ ID NO: 26 - DNA, 1202 bp, Salmonella enterica
  source 1..1202: mol_type = genomic DNA; serotype = Salmonella enterica Typhi
atggcaattaaattagaagtgaagaatctgtataaaatatttggagagcatccgcagcgtgccttcaaatatattgaaaagggactatcgaaagagcaaatactggaaaaaacggggctatcgcttggcgttaaagacgccagtctggccattgaagaaggcgagatatttgtcatcatgggattatccggctcgggtaaatccacaatggtacgccttctcaatcgcctgattgaacccacccgcgggcaggtactgattgacggtgttgatattgccaaaatatcagacgctgagcttcgcgaggtgcgcaggaaaaagattgcgatggtcttccagtcatttgcgctcatgccgcatatgaccgtgctggataatacggcattcggtatggaattagcgggcatcgcggcgcaagagcgtcgcgaaaagcgctggacgccttgcgtcaggtggggcttgagaattacgctcacgcctatccggatgaactttccggtgggatgcgtcagcgtgttgggcttgcccgcgcgctggcaatcaaccctgatatcttattaatggatgaagcgttttccgccctcgatccattaattcgtaccgaaatgcaggatgagctggtgaaattacaggcgaaacatcagcgcaccattgtctttatttcccacgatcttgatgaggctatgcgtattggcgacaggattgccattatgcaaaatggcgaggtcgtacaggttggtacgccggatgagatcctgaataatccggcaaatgattatgtccgcacgttcttccgtggcgtggatattagtcaggtctttagcgccaaagatattgcccgtcgcagtccggtcggcttaattcgtaaaacgccaggttttggtccccgttcggcactgaaattattacaggatgaagaccgtgaatatggttacgtcattgagcgtggcaataaattcgtgggcgtcgtgtccatcgactcattaaaagcggcattaagccaggcgcaagggattgaagcggcgcttatcgacgaccctttagtcgttgatgcgcaaaccccactcagcgagttgctctctcacgtcggccaggcgccctgcgcggtgccggttgtcgatgaagaacaccagtatgttggcattatttcaaaacgtatgttgctacaggctttagatcgcgaggggggtaacaatggctga

SEQ ID NO: 27 - DNA, 588 bp, Salmonella enterica
  source 1..588: mol_type = genomic DNA; serotype = Salmonella enterica Typhi
atgaagtcatctcatttttgtaaactggcagtaactgcatctttagttatgggaattgtctccggcgctcaagccgcgggtagcaacacagcaaaggttactttccttggtaatattgttgattccccctgctctgtcacattggatacggaagatcaaacagtcaatatgggctcaagtatcggtaatggcacgctgagtaatggtaaaacgaccatcaacaatgcccgtacctttcatatcgatcttgagggttgtacctaggctaccgagaaaaatatgaatgtggtattcactacaggtagtggaaccacagcggctacaggcgccacggataatctcgcgctgatgaagactgacggcactggcgctattagcaacataagcctggcaatcggcgatgcaggcaaaaacaatatcaaactgggcgatacctatacacaggccattgcggacctggacagagataccatccttgatgagaagcaaagcctgaatttcaccgcctggctggttggcgcagcaaccggcaccgtaggcacaggtgaattcagcagcgccgccaacgtcactatctcttacctgtaa

SEQ ID NO: 28 - DNA, 873 bp, Salmonella enterica
  source 1..873: mol_type = genomic DNA; serotype = Salmonella enterica Typhi
atgggtgaattttcgacacttcttcagcaaggaaacggctggttcttcattcccagcgccattttattaggtattttgcacgggcttgaaccagggcactccaaaaccatgatggcggcttttatcattgccattaaaggtacggttaaacaggctgtcatgctcggtctggcagcaacgctttctcataccgcgatcgtctggttaatcgcgctgggtgggatgtatcttagccgggcattcaccgcacaatcagtggaaccatggctgcagttaatttctgcgatcattattctgagcaccgcgtgctggatgttctggcggacatgacgaggcgagcagcagtggctggcgggaaaccaccatcacgaccaccatcacgaccaccattacgaccatgaccatgaccatgaccatgaccatgaccatgaccatgaccatgaccatgaccatcatggtcacatacatccggaaggcgcaacgtcaaaagcgtatcaggatgcccatgaacgcgcccatgctgccgatattcaacgccgttttgatggtcaaacagtgaataatggacagatcctgctgttcggcctgaccggagggcttatcccctgtccggctgcgatcaccgttttactgatttgtatccagcttaaagcgtttacgctgggtgccacgatggtgctgagctttagtcttggcctggcattaacgctggtgacggtaggcgttggcgcggcgataagcgttcaacaggcagcaaagcgctggagtggtttttcgacgcttgcccggcgggcgccctatttttcgagcattctgattggtctggtcggcgtgtatatgggaattcatggctataccgggatcatgcagtaa

SEQ ID NO: 29 - DNA, 141 bp, Salmonella enterica
  source 1..141: mol_type = genomic DNA; serotype = Salmonella enterica Typhi
atgccgcactgccaggacaacaccaaacgtgagttcacacatctggttagagtttctctggcttaccgcaaaattgagtgggaacacgtttcaacaggcacttcaggtgctgatgactgacgtgcgccgctggaagcataa

SEQ ID NO: 30 - DNA, 695 bp, Salmonella enterica
  source 1..695: mol_type = genomic DNA; serotype = Salmonella enterica Typhi
tcaatacacctccgaggctaaaaagtagccttcgccatgctgtgtgaccagcagttccggcgtaattttatgacgtaagcggcgaaccaggacatcaatcgtgcggagatccggggtctctacgcggcgggcggaaagcattcgcagcagacgttcgcgatgcagcactttgcctgggttggtcacaaaggccagcagcagctcgtactccgcgcgcgtaagcttaatggcttcaccgttgtgctccagcgtgtgattcatcacgttcaggcagtagccggaaaacatatagcagttttcactggcattttgcggcgtggggcgggccagatcgatacgccacaaaagatttttcacccgcactaccagctcgcgcagttccagcggcttggtgacgtagtcgtccgcgcccatctccagcccgacgatgcggtcgatttggtcgcaacgccccgtcaccagaataatgcccaccgtggagcgttcgcgtagcgccctggtcagcatcaacccgttttcatcggggaggttgatatccagcaggatcagcgaaacgtgctcatgctccatgatgtcacgcgcaggcccgcgccgctgtcggtcaccgaaacgcgatacccctcctgctcaaaataggcctgtaacctggcctgagtaacaggttcatcctcaacaataacaatgtgatgtgacat

SEQ ID NO: 31 - DNA, 53 bp, synthetic construct
  source 1..53: mol_type = other DNA
ccgcggtgcgggtgccagggcgtgcccttgggctccccgggcgcgtactccac

SEQ ID NO: 32 - DNA, 53 bp, synthetic construct
  source 1..53: mol_type = other DNA
ccgcggtgcgggtgccagggcgtgcccgcgggctccccgggcgcgtactccac

SEQ ID NO: 33 - DNA, 53 bp, synthetic construct
  source 1..53: mol_type = other DNA
ccgcggtgcgggtgccagggcgtgcccctgggctccccgggcgcgtactccac

SEQ ID NO: 34 - DNA, 53 bp, synthetic construct
  source 1..53: mol_type = other DNA
ccgcggtgcgggtgccagggcgtgccctagggctccccgggcgcgtactccac

SEQ ID NO: 35 - DNA, 53 bp, synthetic construct
  source 1..53: mol_type = other DNA
ccgcggtgcgggtgccagggcgtgcccatgggctccccgggcgcgtactccac

SEQ ID NO: 36 - DNA, 53 bp, synthetic construct
  source 1..53: mol_type = other DNA
ccgcggtgcgggtgccagggcgtgcccccgggctccccgggcgcgtactccac

SEQ ID NO: 37 - DNA, 42 bp, synthetic construct
  source 1..42: mol_type = other DNA
ggccggcttgtcgacgacggcgttctccgtcgtcaggatcat

SEQ ID NO: 38 - DNA, 42 bp, synthetic construct
  source 1..42: mol_type = other DNA
ggccggcttgtcgacgacggcggcctccgtcgtcaggatcat

SEQ ID NO: 39 - DNA, 42 bp, synthetic construct
  source 1..42: mol_type = other DNA
ggccggcttgtcgacgacggcgctctccgtcgtcaggatcat

SEQ ID NO: 40 - DNA, 42 bp, synthetic construct
  source 1..42: mol_type = other DNA
ggccggcttgtcgacgacggcgtactccgtcgtcaggatcat

SEQ ID NO: 41 - DNA, 42 bp, synthetic construct
  source 1..42: mol_type = other DNA
ggccggcttgtcgacgacggcgatctccgtcgtcaggatcat

SEQ ID NO: 42 - DNA, 42 bp, synthetic construct
  source 1..42: mol_type = other DNA
ggccggcttgtcgacgacggcgccctccgtcgtcaggatcat

SEQ ID NO: 43 - DNA, 152 bp, synthetic construct
  source 1..152: mol_type = other DNA
ccggcttatcggtcagtttcacctgatttacgtaaaaacccgcttcggcgggtttttgcttttggaggggcagaaagatgaatgactgtccacgacgctatacccaaaagaaaaaaaaaaaaccccgcccctgacagggcggggtttttttt

SEQ ID NO: 44 - DNA, 110 bp, synthetic construct
  source 1..110: mol_type = other DNA
tccggcaattaaaaaagcggctaaccacgccgctttttttacgtctgcactcggtaccaaattccagaaaagaggcctcccgaaaggggggccttttttcgttttggtcc

SEQ ID NO: 45 - DNA, 147 bp, synthetic construct
  source 1..147: mol_type = other DNA
ttcagccaaaaaacttaagaccgccggtcttgtccactaccttgcagtaatgcggtggacaggatcggcggttttcttttctcttctcaactcggtaccaaagacgaacaataagacgctgaaaagcgtcttttttcgttttggtcc

SEQ ID NO: 46 - DNA, 109 bp, synthetic construct
  source 1..109: mol_type = other DNA
gctgatgccagaaagggtcctgaatttcagggccctttttttacatggattgctcggtaccaaattccagaaaagagacgctttcgagcgtcttttttcgttttggtcc

SEQ ID NO: 47 - DNA, 86 bp, synthetic construct
  source 1..86: mol_type = other DNA
gatctaactaaaaaggccgctctgcggccttttttcttttcactgtaacaacggaaaccggccattgcgccggttttttttggcct

SEQ ID NO: 48 - DNA, 92 bp, synthetic construct
  source 1..92: mol_type = other DNA
agttaaccaaaaaggggggattttatctcccctttaatttttcctcgcagatagcaaaaaagcgcctttagggcgcttttttacattggtgg

SEQ ID NO: 49 - DNA, 112 bp, synthetic construct
  source 1..112: mol_type = other DNA
ggaaacacagaaaaaagcccgcacctgacagtgcgggctttttttttcgaccaaaggctcggtaccaaattccagaaaagacacccgaaagggtgttttttcgttttggtcc

SEQ ID NO: 50 - DNA, 133 bp, synthetic construct
  source 1..133: mol_type = other DNA
taccaccgtcaaaaaaaacggcgctttttagcgccgtttttatttttcaaccttccaggcatcaaataaaacgaaaggctcagtcgaagactgggcctttcgttttatctgttgtttgtcggtgaacgctctc

SEQ ID NO: 51 - DNA, 90 bp, synthetic construct
  source 1..90: mol_type = other DNA
acatttaataaaaaaagggcggtcgcaagatcgcccttttttacgtatgacacagtgaaaaatggcgcccatcggcgccatttttttatg

SEQ ID NO: 52 - DNA, 85 bp, synthetic construct
  source 1..85: mol_type = other DNA
tgctcgtaccaggcccctgcaatttcaacaggggcctttttttatccaattccatcgggtccgaattttcggaccttttctccgc

SEQ ID NO: 53 - DNA, 90 bp, synthetic construct
  source 1..90: mol_type = other DNA
cttattccataacaaagccgggtaattcccggctttgttgtatctgaacaataaatggatgccctgcgtaagcggggcatttttcttcct

SEQ ID NO: 54 - DNA, 77 bp, synthetic construct
  source 1..77: mol_type = other DNA
agcgtcaaaaggccggattttccggccttttttattaggcagcatgctgccaggtgatccccctggccacctctttt

SEQ ID NO: 55 - DNA, 103 bp, synthetic construct
  source 1..103: mol_type = other DNA
taatcattcttagcgtgaccgggaagtcggtcacgctacctcttctgaagaaacagcaaacaatccaaaacgccgcgttcagcggcgttttttctgcttttct

SEQ ID NO: 56 - DNA, 118 bp, synthetic construct
  source 1..118: mol_type = other DNA
gtgaagtgaaaaatggcgcacattgtgcgccattttttttgtctgccgtttaccgcttctctgaaaatcaacgggcaggtcactgacttgcccgtttttttatcccttctccacaccg

SEQ ID NO: 57 - DNA, 170 bp, synthetic construct
  source 1..170: mol_type = other DNA
tctttaaaaagaaacctccgcattgcggaggtttcgccttttgatactctgtctgaagtaattcttgccgcagtgaaaaatggcgcccatcggcgccatttttttatgcttccattagaaagcaaaaagcctgctagaaagcaggcttttttgaatttggctcctctgac

SEQ ID NO: 58 - DNA, 113 bp, synthetic construct
  source 1..113: mol_type = other DNA
aaagttctgaaaaagggtcacttcggtggcccttttttatcgccacggtttgagcagtgcacttgcttaaaatcccgccagcggcgggattttttattgtccggtttaagaca

SEQ ID NO: 59 - DNA, 166 bp, synthetic construct
  source 1..166: mol_type = other DNA
gcagacaaaaaaaatggcgcacaatgtgcgccatttttcacttcacaggtactattgttttgaattgaaaagggcgcttcggcgccctttttgcatttgttgacggcatatatttgtatatcgaagcgccctgatgggcgctttttttatttaatcgataaccaga

SEQ ID NO: 60 - DNA, 488 bp, synthetic construct
  source 1..488: mol_type = other DNA
ccagatcgttcctcaggtgacctcgagtccggcaattaaaaaagcggctaaccacgccgctttttttacgtctgcactcggtaccaaattccagaaaagaggcctcccgaaaggggggccttttttcgttttggtccccgcggtgcgggtgccagggcgtgcccttgggctccccgggcgcgtactccacgaagttcctatactttctagagaataggaacttcggaataggaactaccgcggtgcgggtgccagggcgtgccctcgggctccccgggcgcgtactccacgtcagaggagccaaattcaaaaaagcctgctttctagcaggctttttgctttctaatggaagcataaaaaaatggcgccgatgggcgccatttttcactgcggcaagaattacttcagacagagtatcaaaaggcgaaacctccgcaatgcggaggtttctttttaaagactgcagcagcgattgagactcagcgaac

SEQ ID NO: 61 - DNA, 431 bp, synthetic construct
  source 1..431: mol_type = other DNA
ccagatcgttcctcaggtgacctcgaggtgaagtgaaaaatggcgcacattgtgcgccattttttttgtctgccgtttaccgcttctctgaaaatcaacgggcaggtcactgacttgcccgtttttttatcccttctccacaccgcggtgcgggtgccagggcgtgcccgtgggctccccgggcgcgtactccacgaagttcctatactttctagagaataggaacttcggaataggaactaccggtgcgggtgccagggcgtgccccagggctccccgggcgcgtactccacggaccaaaacgaaaaaaggcccccctttcgggaggcctcttttctggaatttggtaccgagtgcagacgtaaaaaaagcggcgtggttagccgcttttttaattgccggactgcagcagcgattgagactcagcgaac

SEQ ID NO: 62 - DNA, 407 bp, synthetic construct
  source 1..407: mol_type = other DNA
ccagatcgttcctcaggtgacctcgagagttaaccaaaaaggggggattttatctcccctttaatttttcctcgcagatagcaaaaaagcgcctttagggcgcttttttacattggtggcggtgcgggtgccagggcgtgcccctgggctccccgggcgcgtactccacgaagttcctatactttctagagaataggaacttcggaataggaacttccggtgcgggtgccagggcgtgcccccgggctccccgggcgcgtactccacggaccaaaacgaaaaaacaccctttcgggtgtcttttctggaatttggtaccgagcctttggtcgaaaaaaaaagcccgcactgtcaggtgcgggcttttttctgtgtttccctgcagcagcgattgagactcagcgaac

SEQ ID NO: 63 - DNA, 2051 bp, synthetic construct
  source 1..2051: mol_type = other DNA
aaattctgagcttcattcctgagccttgctctgatgttggccgttccttttgccccgcagccagatcgttcctcaggtgacctcgagtccggcaattaaaaaagcggctaaccacgccgctttttttacgtctgcactcggtaccaaattccagaaaagaggcctcccgaaaggggggccttttttcgttttggtccccgcggtgcgggtgccagggcgtgcccttgggctccccgggcgcgtactccacgaagttcctatactttctagagaataggaacttcggaataggaacttcatgcctttaattaagggcgaccgtcttctatcggtaataacagtccaatctggtgtaacttcggaatcgtccccaattattgaacacccttcggggtgtttttttgtttctggtctaccatctcgttgtgataatagacctgaagtgcctactctggaaaatctttgacagctagctcagtcctaggtataatgctagcagctgtcaccggatgtgctttccggtctgatgagtccgtgaggacgaaacagcctctacaaataattttgtttaaaaagaggagaaatagtccatgcgctgatagtgctagtgtagatcgctactagagccaggcattttatatactggctcgggtaagaactcgcacttcgtggaaacactattatctggtggaagacgtgagcactagtcttggactcctgttgatagatccagtaatgacctcagaactccatctggatttgttcagaacgctcggttgccgccgggcgttttttattggtgagaatccaggggtccccaataattacgatttaaattggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgacttttgtcggctcgacccacgactattgactgctctgagaaagttgattgttacgattagtccggccggccgaagttcctatactttctagagaataggaacttcggaataggaactaccgcggtgcgggtgccagggcgtgccctcgggctccccgggcgcgtactccacgtcagaggagccaaattcaaaaaagcctgctttctagcaggctttttgctttctaatggaagcataaaaaaatggcgccgatgggcgccatttttcactgcggcaagaattacttcagacagagtatcaaaaggcgaaacctccgcaatgcggaggtttctttttaaagactgcagcagcgattgagactcagcgaaccaacaccaataaagtgatctattcgaaccacccggatctggtgcgtccgattgcctcgat

SEQ ID NO: 64 - DNA, 1527 bp, synthetic construct
  source 1..1527: mol_type = other DNA
tttattgcgcagtatgacgccgtcattcaggtgggcggttcaaactatgtcgacttatacccagatcgttcctcaggtgacctcgaggtgaagtgaaaaatggcgcacattgtgcgccattttttttgtctgccgtttaccgcttctctgaaaatcaacgggcaggtcactgacttgcccgtttttttatcccttctccacaccgcggtgcgggtgccagggcgtgcccgtgggctccccgggcgcgtactccacgaagttcctatactttctagagaataggaacttcggaataggaacttcttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaataattaattgagaagttcctatactttctagagaataggaacttcggaataggaactaccggtgcgggtgccagggcgtgccccagggctccccgggcgcgtactccacggaccaaaacgaaaaaaggcccccctttcgggaggcctcttttctggaatttggtaccgagtgcagacgtaaaaaaagcggcgtggttagccgcttttttaattgccggactgcagcagcgattgagactcagcgaacggagcgggcgaaagggttcacgatgatcaaatctgctctcgacagaattcgggaggcgca

SEQ ID NO: 65 - DNA, 1571 bp, synthetic construct
  source 1..1571: mol_type = other DNA
agccaggctgactccgatcgcatcagctattactcgcgcgggtttgaaattgacaactatccagatcgttcctcaggtgacctcgagagttaaccaaaaaggggggattttatctcccctttaatttttcctcgcagatagcaaaaaagcgcctttagggcgcttttttacattggtggcggtgcgggtgccagggcgtgcccctgggctccccgggcgcgtactccacgaagttcctatactttctagagaataggaacttcggaataggaacttctagtagcccgcctaatgagcgggcttttttttaattcccctatttgtttatttttctaaatacattcaaatatgtatccgctcatgagacaataaccctgataaatgcttcaataatattgaaaaaggaagagtatgagcattcagcattttcgtgtggcgctgattccgttttttgcggcgttttgcctgccggtgtttgcgcatccggaaaccctggtgaaagtgaaagatgcggaagatcaactgggtgcgcgcgtgggctatattgaactggatctgaacagcggcaaaattctggaatcttttcgtccggaagaacgttttccgatgatgagcacctttaaagtgctgctgtgcggtgcggttctgagccgtgtggatgcgggccaggaacaactgggccgtcgtattcattatagccagaacgatctggtggaatatagcccggtgaccgaaaaacatctgaccgatggcatgaccgtgcgtgaactgtgcagcgcggcgattaccatgagcgataacaccgcggcgaacctgctgctgacgaccattggcggtccgaaagaactgaccgcgtttctgcataacatgggcgatcatgtgacccgtctggatcgttgggaaccggaactgaacgaagcgattccgaacgatgaacgtgataccaccatgccggcagcaatggcgaccaccctgcgtaaactgctgacgggtgagctgctgaccctggcaagccgccagcaactgattgattggatggaagcggataaagtggcgggtccgctgctgcgtagcgcgctgccggctggctggtttattgcggataaaagcggtgcgggcgaacgtggcagccgtggcattattgcggcgctgggcccggatggtaaaccgagccgtattgtggtgatttataccaccggcagccaggcgacgatggatgaacgtaaccgtcagattgcggaaattggcgcgagcctgattaaacattggtaaagaagttcctatactttctagagaataggaacttcggaataggaactaccggtgcgggtgccagggcgtgcccccgggctccccgggcgcgtactccacggaccaaaacgaaaaaacaccctttcgggtgtcttttctggaatttggtaccgagcctttggtcgaaaaaaaaagcccgcactgtcaggtgcgggcttttttctgtgtttccctgcagcagcgattgagactcagcgaacggttgacggtattccaacgtattttgagtcccgctggaatctgggcgatgcgctaacgga

(19) United States
(12) Patent Application Publication
(10) Pub. No.: US 2026/0357191 A1
(43) Pub. Date: Nov. 12, 2026

(54) SALMONELLA STRAIN WITH CHROMOSOMALLY INTEGRATED LANDING PAD

(71) Applicant: PROKARIUM LIMITED, London (GB)

(72) Inventors: Marc Biarnes Carrera, London (GB); Paola Salerno, London (GB)

(21) Appl. No.: 19/112,206

(22) PCT Filed: Sep. 14, 2023
(86) PCT No.: PCT/EP2023/075364

§ 371 Date: Mar. 14, 2025

(30) Foreign Application Priority Data
Sep. 21, 2022 (EP) 22196895.1

(51) Int. Cl. C12N 1/20; A61K 35/00; A61K 35/74; A61K 39/00; A61K 39/112; A61K 45/06; C12N 15/74

(52) U.S. Cl. CPC C12N 1/20; A61K 35/74; A61K 39/0275; A61K 45/06; C12N 15/74; A61K 2035/11; A61K 2039/522; A61K 2039/523

(57) ABSTRACT

The present invention relates to a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus as defined by any one of SEQ ID NOs: 1-30, or a sequence comprising at least 70% identity to any one of SEQ ID NOs: 1-30. The present invention also relates to a vaccine composition comprising the modified strain and various uses and methods thereof.

### FIELD OF THE INVENTION

The present invention relates to the use of modified Salmonella to deliver heterologous cargo and the methods thereof.

### BACKGROUND

Microorganisms, such as bacteria, can be genetically engineered to carry and deliver heterologous cargo. Many of the current techniques involved in modulation of gene expression are plasmid-based. However, plasmid-based systems have numerous disadvantages associated with them, including introducing cell-to-cell variability, enhanced metabolic burden, reliance on antibiotic resistance genes and removal from the resulting therapeutic strains.

One way in which these problems may be overcome is via the introduction of the desired cargo into the chromosome of the host organism. Kuhlman and Cox (Nucleic Acids Research, 2010, 38:6, e92) disclose a system in which the helper plasmid TKRED catalyses the introduction of a “landing pad” into arbitrary positions in the Escherichia coli chromosome to enable the insertion of heterologous cargo. Such an approach has also been used by Snoeck et al., (Biotechnology and Bioengineering, 2019, 116:364-374), who used serine recombinases to integrate cargo into the Escherichia coli chromosome with landing pads with cognate att sites located at random locations across the genome. Additionally, US 2014/127816 A1 discloses the introduction of recombinase recognition sites in the intergenic regions upstream and downstream of the genes to effectively excise the gene.

However, genome integration also poses numerous challenges. For example, integration of cargo into the chromosome is laborious and challenging for very large constructs. Additionally, any genomic modification/integration can affect strain physiology in an unpredictable manner and thus alter its effectiveness as a potential therapeutic strain, especially where genes are modified at intergenic sites (as in US 2014/127816 A1) which has the potential side-effect of disrupting or otherwise impacting the surrounding active genes. Finally, the amount of transcript an introduced circuit can produce is dependent on its location in the genome. As such, random integration can lead to uncontrollable and unreliable levels of the desired cargo being produced. Accordingly, there is a need in the field for improved ways in which cargo can be introduced into the chromosome of a host organism.

### SUMMARY OF INVENTION

The inventors of the present invention have surprisingly found that the generation of a Salmonella strain comprising at least one chromosomally integrated synthetic polynucleotide sequence in a pre-determined genomic location not only enables for the effective and rapid introduction of any desired cargo into said strain, but also allows for the cargo to be introduced into genomic locations of known transcriptional strength, allowing for more controlled delivery of various cargo. Further, the present inventors have identified specific pseudogenomic genomic locations, which comprise regions of inactive DNA, for the insertion of these chromosomally integrated sequences that allow for normal bacterial cell physiology, and its capability as a therapeutic strain, to be minimally affected. By introducing “landing pads” into pseudogenes within the bacterial chromosome (inactive DNA regions), the present inventors have found a way in which new genes and functionalities can be introduced into a Salmonella strain without affecting surrounding genes, thus allowing for normal bacterial cell physiology, and any therapeutic capability of the bacterial strain to be minimally affected.

Accordingly, in a first aspect, the present invention discloses a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

In a second aspect, the present invention discloses a vaccine composition comprising the modified live attenuated strain herein disclosed.

In a third aspect, the present invention discloses the modified live attenuated strain herein disclosed for use in the treatment of cancer.

In a fourth aspect, the present invention discloses the modified live attenuated strain herein disclosed for use in the treatment of infectious disease.

In a fifth aspect, the present invention discloses the modified live attenuated strain herein disclosed for use in the treatment of an autoimmune disease or disorder.

In a sixth aspect, the present invention discloses a method of treating, inhibiting or controlling a neoplastic disease, or an infectious disease, in a subject, wherein the method comprises administering to the subject a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

In a seventh aspect, the present invention discloses a method for modifying a live attenuated strain of Salmonella, said method comprising inserting a synthetic polynucleotide sequence into a pre-determined pseudogenomic location of the live attenuated strain of Salmonella, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

### BRIEF DESCRIPTION OF DRAWINGS

FIG. 1 shows the alignment of the Salmonella ZH9 strain sequence compared to the reference genome of the Salmonella Ty21a strain.

FIG. 2 shows the location of pseudo-genes within the genome of the ZH9 strain.

FIG. 3 shows a possible design of the synthetic sequence herein disclosed to be chromosomally integrated into the Salmonella strain.

FIG. 4 shows the reproducibility of small-scale integration rounds in the Salmonella ZH9 strain.

FIG. 5 shows the optimisation of the conditions required for successful integration of the synthetic sequence into the chromosome of the Salmonella ZH9 strain.

FIG. 6 shows the number of successful integrands in each integration round, validation of integration via ColonyPCT and the final genomic location of the synthetic sequence in the Salmonella ZH9 genome.

FIG. 7 shows the optimisation of the auxiliary plasmid curing conditions.

FIG. 8 shows the growth curve and doubling time of strains with the chromosomally integrated synthetic sequence grown in either complex media (vegan Lysogeny Broth) or minimal media (M9 media) for 12 h at 37 °C.

FIG. 9 shows the design of a possible shuttle vector used to introduce cargo into the chromosomally integrated synthetic sequence.

FIG. 10 shows the induction of protein production in the Salmonella ZH9 strain using the shuttle vector design of FIG. 9.

FIG. 11 shows the induction effect on strain growth in the Salmonella ZH9 strain using the shuttle vector design of FIG. 9.

FIG. 12 shows the expression of the introduced cargo as a function of its genomic location being determined via two assays: the chloramphenicol acetyltransferase (CAT) assay and the mScarlet assay. The correlation between these two independent assays is also shown.

FIG. 13 shows the mScarlet fluorescence at three thresholds (OD600~0.3, 0.5, 1.0) of a Salmonella ZH9 strain successfully integrated with the synthetic sequence herein disclosed.

FIG. 14 shows the ranking of various strains integrated with the synthetic sequence herein disclosed at different genomic locations by their relative transcriptional strength.

FIGS. 15 and 16 show the procedure followed when subculturing strains A-C, containing up to three landing pads, during four consecutive days and the genome sequencing results at each of the landing pad positions on day 4. No alterations were observed in any of these.

FIGS. 17 and 18 show the plasmids used to integrate a fluorescent protein (mScarlet) into the multi-landing pad Strain C (three landing pads) and the curing of each of the auxiliary plasmids. The remaining strain had no antibiotic resistance and retained the cargo.

### DETAILED DESCRIPTION

In order that the present invention may be more readily understood, certain terms are first defined. Additional definitions are set forth throughout the detailed description.

As used herein, the term “attenuated” refers to a bacterium that has been genetically modified so as not to cause illness in a human or animal subject/model. Accordingly, in the context of the present invention, the term refers to the alteration of a Salmonella bacterium to reduce its pathogenicity, rendering it harmless to the host, whilst maintaining its viability. This method is commonly used in the development of vaccines due to its ability to elicit a highly specific immune response whilst maintaining an acceptable safety profile. Attenuation can be achieved via a number of methods, examples include, but are not limited to, passing the pathogens under in vitro conditions until virulence is lost, chemical mutagenesis and genetic engineering techniques. The attenuated Salmonella strain herein disclosed is a live attenuated Salmonella strain.

As used herein, the terms “chromosomally integrated” and “chromosomal integration” are used interchangeably and refers to the stable incorporation of a nucleic acid sequence into the chromosome of a host cell i.e., a Salmonella bacterium. The nucleic acid sequence may be incorporated into the chromosome via a number of methods. In the context of the present invention, the nucleic acid sequence to be incorporated into the chromosome of the Salmonella bacterium is the “chromosomally integrated synthetic polynucleotide sequence” or “landing pad” sequence. Preferably, the nucleic acid sequence encoding the “chromosomally integrated synthetic polynucleotide sequence” or “landing pad” sequence is incorporated into the chromosome using the classical recombineering protocol disclosed in Datsenko and Wanner, 2000 (PNAS, 97(12): 6640-6645). Briefly, the cells are first transformed with an auxiliary plasmid, then with a dsDNA cassette, and then the auxiliary plasmid is cured and removed. The antibiotic introduced in the recombination is removed by using the protein FLP that excised it upon recognition of the FRT elements. Integration can subsequently be validated via PCR reaction designed to yield amplicon (~500 bp) only in the case of successful recombination.

As used herein, the terms “pseudogenomic”, “pseudogenic” and “pseudogenomic location” are used interchangeably and refers to the location of an inactive gene or DNA sequence within the host genome i.e., the genome of a Salmonella strain. Such inactive genes are referred to as “pseudogenes”. Pseudogenes may further be referred to as “non-functional” or “inactive” regions or segments of DNA. The inventors of the present invention have identified pseudogenes. Pseudogenes are regions of DNA that may or may not have once encoded functional genes but have since been deactivated through mutations, such as premature stop codons. Pseudogenic regions of DNA contrast to intergenic regions of DNA. Intergenic regions of DNA are locations of DNA which comprise active or functional genes. Intergenic regions are likely to be active with promoters, terminators, or anti-sense RNA. The present invention introduces new genes and functionalities into pseudogenic regions of DNA, as opposed to integrating new genes into intergenic regions, which has the potential unpredictable side-effect of disrupting or otherwise affecting or interfering with the surrounding active/functional genes. For the avoidance of doubt, the landing pads disclosed herein are to be inserted into pseudogenomic regions, i.e., in between stop and start codons within the pseudogene.

As used herein, the terms “polynucleotide” and “polynucleotide sequence” are used interchangeably and refer to a polymeric compound comprising covalently linked nucleic acids. The term “nucleic acid” includes both DNA and RNA.

As used herein, the terms “polypeptide”, “peptide” and “protein” are used interchangeably and refer to a polymeric form of amino acids of any length, which can include coded and non-coded amino acids, chemically or biochemically modified and/or derivatized amino acids.

As used herein, the term “defined recombination site” refers to the Salmonella strains herein disclosed comprising short, specific DNA sequences (sites) at which genetic recombination can take place, thus allowing the introduction of a heterologous polynucleotide sequence.

As used herein, the term “genomic locus” refers to the location of a specific gene or synthetic sequence within the genome of the Salmonella bacterium.

As used herein, the term “pre-determined genomic location” refers to the introduction of the synthetic polynucleotide sequence into pre-planned, specific genomic locations of the Salmonella strain chromosome. The synthetic polynucleotide sequences of the present invention are not integrated randomly into the bacterial chromosome, as has previously been done in the art.

As used herein, the terms “sequence homology” and “sequence identity” are used interchangeably and refer to the percentage of residues in the compared sequences that are the same when the sequences are aligned. To calculate % sequence homology/identity of any of the sequences herein disclosed, sequence comparison software can be used, for example, using the default settings on the BLAST software package (V2.10.1).

As used herein, the term “genetically engineered” refers to a bacterium, such as a Salmonella bacterium, that has been genetically modified or “engineered” such that it is altered with respect to the naturally occurring cell. Such genetic modification may for example be the incorporation of additional genetic information into the cell, modification of existing genetic information or indeed deletion of existing genetic information. This may be achieved, for example, by way of transfection of a recombinant plasmid into the cell or modifications directly to the bacterial genome.

By “inactivating mutations”, we mean modifications of the natural genetic code of a particular gene or gene promoter associated with that gene, such as modification by changing the nucleotide code or deleting sections of nucleotide or adding non-coding nucleotides or non-natural nucleotides, such that the particular gene is either not transcribed or translated appropriately or is expressed into a non-active protein such that the gene's natural function is abolished or reduced to such an extent that it is not measurable. Thus, the mutation of the gene inactivates that gene's function or the function of the protein which that gene encodes.

As used herein, the term “transcriptional strength” refers to the extent at which a gene at a specific genomic location is transcribed from DNA to RNA. In the context of the present invention, the “gene” is the newly introduced heterologous polynucleotide sequence introduced into the chromosomally integrated sequence. The transcriptional strength may be described as having a low or high transcriptional strength. The skilled person will readily recognise that the desired transcriptional strength will depend on the cargo to be delivered to the subject. For example, there may be situations where tight regulation of the cargo to be delivered would be highly desirable, i.e., when the cargo to be delivered is of a toxic nature.

The modified Salmonella strains herein disclosed are designed such that heterologous genetic material can be easily introduced into the Salmonella strain and subsequently delivered to the target subject in a reliable and efficient manner. As such, the strains herein disclosed may act as a carrier strain for the delivery of a variety of different “cargo”. As such, when the strains herein disclosed comprise the heterologous genetic material, said strains are said to be “recombinant”. Accordingly, in one embodiment of the present invention, the modified live attenuated strain of Salmonella herein disclosed is a recombinant strain of Salmonella.

As used herein, the term “immunogenic” refers to a substance which elicits an immune response within the subject. The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of unwanted cells, for example, cancerous cells and/or microbes associated with infectious disease.

The terms “tumour,” “cancer” and “neoplasia” are used interchangeably and refer to a cell or population of cells whose growth, proliferation or survival is greater than growth, proliferation, or survival of a normal counterpart cell, e.g., a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations, or regions within the subject, in which the sites, locations or regions are distinct from the primary tumour or cancer.

The terms “effective amount” or “pharmaceutically effective amount” refer to a sufficient amount of an agent to provide the desired biological or therapeutic result i.e., the amount of the Salmonella bacterium herein described to achieve the desired effect. That result can be reduction, amelioration, palliation, lessening, delaying, and/or alleviation of one or more of the signs, symptoms, or causes of a disease, or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development or prolong survival or induce stabilisation of the cancer or tumour. In reference to infectious disease, an effective amount may comprise an amount sufficient to reduce the viral or bacterial load of the subject.

In some embodiments, a therapeutically effective amount is an amount sufficient to prevent or delay recurrence. This may be particularly advantageous in cancer patients. A therapeutically effective amount can be administered in one or more administrations. In the context of cancer, the therapeutically effective amount of the drug or combination may result in one or more of the following: (i) reduction in the number of cancer cells; (ii) reduction in tumour size; (iii) inhibition, retardation, or slowing to some extent and preferably stopping cancer cell infiltration into peripheral organs; (iv) inhibition (i.e., slowing to some extent and preferably stopping) tumour metastasis; (v) inhibiting tumour growth; (vi) preventing or delaying occurrence and/or recurrence of tumour; and/or (vii) relieving to some extent one or more of the symptoms associated with the cancer.

For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects.

A therapeutically effective amount of a therapeutic compound can decrease symptoms, or otherwise ameliorate symptoms completely in a subject. One of ordinary skill in the art would be able to determine such amounts based on such factors as the subject's size, the severity of the subject's symptoms, and the particular composition or route of administration selected.

The term “vaccine composition” and “vaccine” are used interchangeably and refers to a biological preparation that provides active acquired immunity to a particular infectious disease. Typically, the vaccine contains an agent, or “foreign” agent, that resembles the infection-causing microorganism. Such a foreign agent would be recognised by a vaccine-receiver's immune system, which in turn would destroy said agent and develop “memory” against the virus, inducing a level of lasting protection against future viral infections from the same or similar sub-species. Through the route of vaccination, including those vaccine compositions of the present invention, it is envisaged that once the vaccinated subject again encounters the same microorganism of which said subject was vaccinated against, the individual's immune system may thereby recognise said microorganism isolate and elicit a more effective defence against infection. The active acquired immunity that is induced in the subject as a result of the vaccine may be humoral and/or cellular in nature. In the context of the present invention, the “foreign” agent may be part of a virus, a bacterium, a fungi, or a parasite. Alternatively, the “foreign” agent may be a cancer antigen, for example, a tumour-associated antigen.

As used herein, the term “subject” is intended to include human and non-human animals. The methods are particularly suitable for treating human patients having a disorder that can be treated by augmenting the immune response. In a particular embodiment, the methods are particularly suitable for treatment of cancer in vivo.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%. When particular values are provided in the application and claims, unless otherwise stated, the meaning of “about” should be assumed to be within an acceptable error range for that particular value.

The inventors of the present invention have developed a standardised methodology in which a variety of cargo can be introduced into specific chromosomal regions of different Salmonella strains, said chromosomal regions having known transcriptional strength whilst at the same time having no or minimal effect on strain viability and/or therapeutic effect. Accordingly, the modified strains herein disclosed allow for cargo of differing transcriptional requirements to be introduced, therefore allowing the strains herein disclosed to be suitable for use in a range of therapeutic applications. Such an approach has multiple advantages over plasmid-based expression systems, for example, reduced cell-to-cell variability, reduced metabolic burden, reduced reliance on antibiotic resistance genes and permanent expression within the strains.

Accordingly, in a first aspect, the present invention discloses a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30. These loci are detailed in Table 1.

The pseudogenomic locations of the present invention are provided for in Table 1. In one embodiment, the pseudogenomic locations of the present invention comprise 70% sequence identity with the genomic locations provided for in Table 1. In one embodiment, the pseudogenomic locations of the present invention comprise 75% sequence identity with the genomic locations provided for in Table 1. In one embodiment, the pseudogenomic locations of the present invention comprise 80% sequence identity with the genomic locations provided for in Table 1. In one embodiment, the pseudogenomic locations of the present invention comprise 85% sequence identity with the genomic locations provided for in Table 1. In one embodiment, the pseudogenomic locations of the present invention comprise 90% sequence identity with the genomic locations provided for in Table 1. In one embodiment, the pseudogenomic locations of the present invention comprise 95% sequence identity with the genomic locations provided for in Table 1. In one embodiment, the pseudogenomic locations of the present invention comprise 96% sequence identity with the genomic locations provided for in Table 1. In one embodiment, the pseudogenomic locations of the present invention comprise 97% sequence identity with the genomic locations provided for in Table 1. In one embodiment, the pseudogenomic locations of the present invention comprise 98% sequence identity with the genomic locations provided for in Table 1. In one embodiment, the pseudogenomic locations of the present invention comprise 99% sequence identity with the genomic locations provided for in Table 1.

Accordingly, in one embodiment, the chromosomally integrated synthetic polynucleotide sequence is located within at least one genomic locus defined by any one of SEQ ID NOs: 1-30, or a sequence comprising at least 75% identity to any one of SEQ ID NOs: 1-30; at least 80% identity to any one of SEQ ID NOs: 1-30; at least 85% identity to any one of SEQ ID NOs: 1-30; at least 90% identity to any one of SEQ ID NOs: 1-30; at least 91% identity to any one of SEQ ID NOs: 1-30; at least 92% identity to any one of SEQ ID NOs: 1-30; at least 93% identity to any one of SEQ ID NOs: 1-30; at least 94% identity to any one of SEQ ID NOs: 1-30; at least 95% identity to any one of SEQ ID NOs: 1-30; at least 96% identity to any one of SEQ ID NOs: 1-30; at least 97% identity to any one of SEQ ID NOs: 1-30; at least 98% identity to any one of SEQ ID NOs: 1-30; or at least 99% identity to any one of SEQ ID NOs: 1-30. It will be understood that the chromosomally integrated synthetic polynucleotide sequence may be located within at least one genomic locus defined by any one of one of SEQ ID NOs: 1-30 (i.e., one, more than one, or any combination thereof).

As used herein, the terms “sequence homology” and “sequence identity” are used interchangeably and refer to the number of identical residues over a defined length in a given alignment of a DNA sequence, RNA sequence or amino acid sequence. To calculate % sequence identity of any of the sequences herein disclosed, sequence comparison software can be used, for example, using the default settings on the BLAST software package (V2.10.1).

The “chromosomally integrated synthetic polynucleotide sequence” may also be referred to as a “landing pad” (LP), allowing for the fast and efficient integration of any desired cargo into a region of known transcriptional strength in the Salmonella genome, thus allowing for controlled delivery of said cargo. The synthetic polynucleotide sequence is inserted into a pre-determined pseudogenomic location. As described above, knowing where the sequence will be inserted enables a high level of control over the subsequent level of transcription of the introduced cargo, avoiding the unpredictable nature of previous methods of chromosomal integration. Previous issues with methods of chromosomal integration include that random integration may interfere with the host physiology in an unpredictable manner and therefore alter its effectiveness as a potential therapeutic strain. The sequence of the present invention is inserted into at least one pseudogenomic location within the Salmonella genome. Pseudogenomic locations are genomic locations of the Salmonella genome in which there are stretches of DNA that used to contain coding regions that were lost due to mutations and therefore stopped expression of that particular gene.

The “chromosomally integrated synthetic polynucleotide sequence” or “landing pad” may be incorporated into the Salmonella chromosome via any appropriate method known to the skilled person. In one specific embodiment, the “chromosomally integrated synthetic polynucleotide sequence” or “landing pad” sequence is incorporated into the Salmonella chromosome using the classical recombineering protocol disclosed in Datsenko and Wanner, 2000 (PNAS, 97(12): 6640-6645), where the cells are first transformed with an auxiliary plasmid and then with dsDNA cassette. The auxiliary plasmid is subsequently cured and removed. The antibiotic introduced in the recombination is removed by using the protein FLP that excised it upon recognition of the FRT elements. The plasmid expressing FLP is subsequently cured and removed.

For example, the “chromosomally integrated synthetic polynucleotide sequence” or “landing pad” sequence may be introduced into the Salmonella chromosome via the following method. An auxiliary plasmid containing genes beta, gam, and exo may be introduced into the Salmonella strain ZH9 by electroporation. A single clone may be grown overnight in LB media supplemented with appropriate antibiotic and aromatic amino acids. The sample may be diluted 1:500 in fresh LB with antibiotic and amino acids as well as Arabinose to induce the auxiliary gene expression. Samples may be grown to mid-log exponential phase and then made electrocompetent. Samples may be electroporated with a double-stranded DNA cassette. Integration may be confirmed by colony PCR using primers outside of the integration region. Successful recombinants were then grown at 43 °C without antibiotic selection to induce plasmid curing. This may be validated by replica plating of single clones in media with and without antibiotics. Final antibiotic resistance within the chromosome may be removed by transforming with plasmid pCP20 (which contains FLP) and then the plasmid may be cured.

It is noted that in the context of the present invention, plasmids are needed both for the initial introduction of the “chromosomally integrated synthetic polynucleotide sequence” or “landing pad” sequence into the genome, and for the initial introduction of the desired cargo into the chromosome. However, the present invention avoids the need to use plasmids for the Salmonella strain to contain the final therapeutic cargo (e.g., expressing a therapeutic protein). In this later context, expression is influenced by plasmid number (which is variable within a population), can introduce cell burden, and typically requires antibiotic resistance markers). The strains disclosed herein overcome the above disadvantages by having the cargo in the chromosome, as all auxiliary plasmids are cured from them.

In one embodiment, the chromosomally integrated synthetic polynucleotide sequence may be located within the sequence of the known genes presented in Table 1. Said genes may be selected from the list comprising ratB, mglA. pbpG, wcaD, wcak, treA, hvaA, astA, eha, dbpA, Fhue, yceJ, hpcC, fepE, ybbW, ushA, riC, proV, steA or torA.

Whilst the genomic loci of Table 1 are loci of a Salmonella enterica serovar strain, specifically the ZH9 Salmonella strain, it is understood that the present invention is not limited by the specific pseudogenes present in these genomic locations in the ZH9 strain. As such, the present invention also provides for the chromosomally synthetic polynucleotide sequence being located within at least one genomic locus defined by any one of SEQ ID NOs: 1-30, or a sequence comprising at least 70%, at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs: 1-30. The skilled person would readily understand that whilst the specific pseudogenes present in Salmonella strains may differ, the genomic regions associated with these regions of inactive DNA are conserved between different strains. Accordingly, in each Salmonella strain, there will be a genomic region, which includes any one of the specific loci of Table 1, as well as inactive regions of DNA on either side of said loci.

Jacobsen et al., 2011 (Microb Ecol, 62:487-504) define a Pan-genome sequence for Salmonella enterica strains, demonstrating that not only is there a high degree of homology between Salmonella strains, but that variation between different strains was primarily seen in Salmonella Pathogenicity Islands. Furthermore, the same study went further and showed genes unique to each serotype (see S2 supplementary materials). None of these identified genes are present in Table 1, demonstrating the applicability of the invention herein disclosed across various Salmonella strains.

TABLE 1
(Genomic Loci derived from Salmonella enterica
Typhi Ty2 genome; GenBank ID: AE014613):

| Locus | Name of Gene | SEQ ID NO |
|---|---|---|
| t0342 | ratB | 1 |
| t0365 | Unknown | 2 |
| t0469 | Unknown | 3 |
| t0666 | mglA | 4 |
| t0687 | pbpG | 5 |
| t0760 | wcaD | 6 |
| t0771 | wcaK | 7 |
| t1007 | Unknown | 8 |
| t1081 | treA | 9 |
| t1090 | hyaA | 10 |
| t1183 | astA | 11 |
| t1385 | eha | 12 |
| t1450 | Unknown | 13 |
| t1559 | dpbA | 14 |
| t1607 | Unknown | 15 |
| t1666 | Unknown | 16 |
| t1716 | Fhue | 17 |
| t1762 | yceJ | 18 |
| t1817 | hpcC | 19 |
| t1838 | Unknown | 20 |
| t2236 | Unknown | 21 |
| t2279 | fepE | 22 |
| t2339 | ybbW | 23 |
| t2366 | ushA | 24 |
| t2379 | priC | 25 |
| t2706 | proV | 26 |
| t2856 | steA | 27 |
| t2934 | Unknown | 28 |
| t3031 | Unknown | 29 |
| t3694 | torR | 30 |

The chromosomally integrated synthetic sequence comprises at least one defined recombination site to allow the introduction of cargo. The methods by which this may be achieved are well known to the skilled person, for example, via a shuttle plasmid or via split plasmid (shuttle and auxiliary) methodology. Recombination sites are well known in the art and in the context of the present invention comprise defined sequences on two different molecules (i.e., the chromosomally integrated synthetic sequence in the Salmonella genome and the vector carrying the cargo) to enable the integration of heterologous cargo into the modified Salmonella strain herein described. The “cargo” may be heterologous DNA or heterologous RNA.

The defined recombination site may be any suitable recombination site for the purpose herein disclosed. However, preferably the defined recombination site is an attB recombination site. Each attB site comprises a cleavage region, typically comprising 2 bp, which confers the specificity of said site. For example, the cleavage site may be TT, GC, CT, TA, AT, or CC, all of which are orthogonal in nature. The shuttle vector containing the heterologous cargo to be introduced into the Salmonella genome further contains cognate attP sites flanking the cargo, which allows for its insertion into the chromosomally inserted synthetic sequence within the Salmonella genome in a polar fashion (i.e., attB_TT will only react with attP_TT).

In order to mediate the recognition of the sites on the chromosomally integrated synthetic sequence and the shuttle vector carrying the cargo, enzymes known as recombinases are required. The skilled person will readily understand that the chosen recombinase will depend on the chosen recombination sites. The chosen recombinase may be inducible or constitutively expressed. In a preferred embodiment, the at least one defined recombination site is an attB recombination site and the recombinase is a serine recombinase. In one embodiment, the serine recombinase is selected from the list comprising PhiC31, Bxbl, TG1, TP901, A118, SPBc, WB, PhiBT1, Phi370.1, BL3, FC1, K38, R4, PhiRV or MR11. In a preferred embodiment, the serine recombinase is PhiC31 or Bxb1. In a more preferred embodiment, the serine recombinase is PhiC31. Accordingly, in one preferred embodiment, the attB recombination site is a PhiC31 attB recombination site according to any one of SEQ ID NOs: 31 to 36. In another preferred embodiment, the attB recombination site is a Bxbl attB recombination site according to any one of SEQ ID NOs: 37 to 42. As outlined above, the shuttle vector containing the heterologous cargo to be introduced into the Salmonella genome further contains cognate attP sites flanking the cargo, which allows for its insertion into the chromosomally inserted synthetic sequence within the Salmonella genome in a polar fashion (i.e., attB_TT will only react with attP_TT, attB_GC will only react with attP_GC and so on-said sites are indicated in bold in the sequences provided at the end of the description).

It is desirable for the expression of the serine recombinase to be tightly controlled to prevent leaky expression of the recombinase whilst also robust enough to allow for reproducible, high throughput loading of cargo into strains. Any expression system which is capable of achieving the above can be used in the present invention. For example, the expression system may be selected from the group comprising a Lacl expression system, a TetR expression system, a Betl expression system or a PhIF expression system (Meyer et al., Nature Chemical Biology, 15:196-204, 2019). In a preferred embodiment, the expression system is a TetR expression system.

The inserted chromosomally integrated synthetic polynucleotide sequence may further comprise insulator regions flanking (i.e., positioned either side) the defined recombination site. The insulator regions comprise strong bi-directional terminators and are therefore important to prevent any transcriptional machinery from interfering with the introduced synthetic sequence and vice versa. As used herein, the term “bi-directional terminator” refers to a polynucleotide sequence that can terminate RNA polymerase transcription in either the sense or antisense direction. Any insulator region capable of achieving the above may be used. Preferably, the insulator regions may have the sequences according to any one of SEQ ID NOs: 43-59, or a sequence comprising at least 70% identity to said sequences. Even more preferably, the insulator region may have a sequence according to SEQ ID NO: 44 (DT5), SEQ ID NO: 57 (DT101), SEQ ID NO: 56 (DT100), SEQ ID NO: 48 (DT42) or SEQ ID NO: 49 (DT54). In yet an even more preferred embodiment, the chromosomally integrated synthetic sequence may be flanked by the insulator regions according to SEQ ID NO: 44 (DT5) and SEQ ID NO: 57 (DT101), or a sequence comprising at least 70% identity to said sequences. Alternatively, the chromosomally integrated synthetic sequence may be flanked by the insulator regions according to SEQ ID NO: 56 (DT100) and SEQ ID NO: 44 (DT5), or a sequence comprising at least 70% identity to said sequences. Alternatively, the chromosomally integrated synthetic sequence may be flanked by the insulator regions according to SEQ ID NO: 48 (DT42) and SEQ ID NO: 49 (DT54), or a sequence comprising at least 70% identity to said sequences. The insulator region may have a sequence having at least 70%, at least 75%, at least 80%, at least 85%, at least 90%, at least 91%, at least 92%, at least 93%, at least 94%, at least 95%, at least 96%, at least 97%, at least 98%, at least 99% to any one of the sequences according to SEQ ID NOs: 43-59. It is noted that during any pre-clinical testing and experiments, the chromosomally integrated synthetic sequence may also comprise an antibiotic marker to select clones with successful integration. Accordingly, two FRT sites, positioned either side of the antibiotic marker will also be present as a way to remove the antibiotic marker upon clonal confirmation. In one embodiment, the antibiotic marker is removed using a FLP recombinase that recognises the FRT sites and removes the DNA located in-between the two FRT sites. The remaining sequence is referred to as a “scar” sequence. The antibiotic marker may be selected from the list comprising CmR, CarbR or KanR. Accordingly, in one embodiment, the present invention provides a modified live attenuated strain of Salmonella, said strain comprising at least one chromosomally integrated synthetic polynucleotide sequence inserted into a pre-determined pseudogenomic location, said synthetic polynucleotide sequence comprising a FRT site, a first attB attachment site placed upstream of the FRT site and a second attB attachment site located downstream of the FRT site, wherein the first attB attachment site is positioned adjacent to a 5′ terminator region and the second attB attachment site is positioned adjacent to a 3′ terminator region. The modified live attenuated strain of Salmonella may further contain the insulator regions, as described above.

In order for integration of the synthetic sequence into the Salmonella genome, recombination overhangs may be added to the 5′ and 3′ ends of the sequence specific for each location of interest using two standardised amplification sites.

The modified strain herein disclosed may comprise more than one chromosomally integrated synthetic polynucleotide sequence. The modified strain herein disclosed may comprise two integrated synthetic polynucleotide sequences. The modified strain herein disclosed may comprise three integrated synthetic polynucleotide sequences. The modified strain herein disclosed may comprise four integrated synthetic polynucleotide sequences. The modified strain herein disclosed may comprise five integrated synthetic polynucleotide sequences. The modified strain herein disclosed may comprise six integrated synthetic polynucleotide sequences. In one preferred embodiment, the modified strain herein discloses comprises three integrated synthetic polynucleotide sequences. The different synthetic polynucleotide sequences may be used to introduce cargo of the same type in different genomic locations (for example, genomic locations of differing transcriptional strength), or may be used to introduce multiple types of cargo into the same strain.

The modified strain herein disclosed may comprise a single chromosomally integrated synthetic polynucleotide sequence. In a preferred embodiment, the modified strain herein disclosed may comprise a single chromosomally integrated synthetic polynucleotide according to SEQ ID NO: 60. Alternatively, the modified strain herein disclosed may comprise multiple chromosomally integrated synthetic polynucleotide sequences. In order to minimise internal recombination events, where the modified strain comprises multiple chromosomally integrated synthetic polynucleotide sequences, the synthetic sequences may comprise different attB sites and/or terminator regions. In a preferred embodiment, the modified strain herein disclosed may comprise multiple chromosomally integrated synthetic polynucleotide sequences according to SEQ ID NO: 60, 61, 62, or any combination thereof.

Accordingly, in one embodiment, the chromosomally integrated synthetic polynucleotide sequence may comprise a sequence according to SEQ ID NO: 60, 61, 62, or a sequence comprising at least 70% identity to SEQ ID NO: 60, 61, 62, or any combination thereof. For example, the chromosomally integrated synthetic polynucleotide sequence may comprise a sequence according to SEQ ID NO: 60, 61, 62, or a sequence comprising at least 75% identity to SEQ ID NO: 60, 61, 62; the chromosomally integrated synthetic polynucleotide sequence may comprise a sequence according to SEQ ID NO: 60, 61, 62, or a sequence comprising at least 80% identity to SEQ ID NO: 60, 61, 62; the chromosomally integrated synthetic polynucleotide sequence may comprise a sequence according to SEQ ID NO: 60, 61, 62, or a sequence comprising at least 85% identity to SEQ ID NO: 60, 61, 62; the chromosomally integrated synthetic polynucleotide sequence may comprise a sequence according to SEQ ID NO: 60, 61, 62, or a sequence comprising at least 90% identity to SEQ ID NO: 60, 61, 62; the chromosomally integrated synthetic polynucleotide sequence may comprise a sequence according to SEQ ID NO: 60, 61, 62, or a sequence comprising at least 91% identity to SEQ ID NO: 60, 61, 62; the chromosomally integrated synthetic polynucleotide sequence may comprise a sequence according to SEQ ID NO: 60, 61, 62, or a sequence comprising at least 92% identity to SEQ ID NO: 60, 61, 62; the chromosomally integrated synthetic polynucleotide sequence may comprise a sequence according to SEQ ID NO:60, 61, 62, or a sequence comprising at least 93% identity to SEQ ID NO: 60, 61, 62; the chromosomally integrated synthetic polynucleotide sequence may comprise a sequence according to SEQ ID NO: 60, 61, 62, or a sequence comprising at least 94% identity to SEQ ID NO: 60, 61, 62; the chromosomally integrated synthetic polynucleotide sequence may comprise a sequence according to SEQ ID NO: 60, 61, 62, or a sequence comprising at least 95% identity to SEQ ID NO: 60, 61, 62; the chromosomally integrated synthetic polynucleotide sequence may comprise a sequence according to SEQ ID NO: 60, 61, 62, or a sequence comprising at least 96% identity to SEQ ID NO: 60, 61, 62; the chromosomally integrated synthetic polynucleotide sequence may comprise a sequence according to SEQ ID NO: 60, 61, 62, or a sequence comprising at least 97% identity to SEQ ID NO: 60, 61, 62; the chromosomally integrated synthetic polynucleotide sequence may comprise a sequence according to SEQ ID NO: 60, 61, 62, or a sequence comprising at least 98% identity to SEQ ID NO: 60, 61, 62; or the chromosomally integrated synthetic polynucleotide sequence may comprise a sequence according to SEQ ID NO: 60, 61, 62, or a sequence comprising at least 99% identity to SEQ ID NO: 60, 61, 62.

The modified strain herein disclosed may comprise the synthetic sequence according to SEQ ID NO: 60. The modified strain herein disclosed may comprise the synthetic sequence according to SEQ ID NO: 60, wherein SEQ ID NO: 60 is flanked by the insulator regions according to SEQ ID NOs: 44 and 57. The modified strain herein disclosed may comprise the synthetic sequence according to SEQ ID NO: 61. The modified strain herein disclosed may comprise the synthetic sequence according to SEQ ID NO: 61, wherein SEQ ID NO: 61 flanked by the insulator regions according to SEQ ID NOs: 56 and 44. The modified strain herein disclosed may comprise the synthetic sequence according to SEQ ID NO: 62. The modified strain herein disclosed may comprise the synthetic sequence according to SEQ ID NO: 62, wherein SEQ ID NO: 62 is flanked by the insulator regions according to SEQ ID NOs: 48 and 49.

The modified strain herein disclosed may comprise the synthetic sequence according to SEQ ID NO: 60 and the synthetic sequence according to SEQ ID NO: 61. The modified strain herein disclosed may comprise the synthetic sequence according to SEQ ID NO: 60 and the synthetic sequence according to SEQ ID NO: 62. The modified strain herein disclosed may comprise the synthetic sequence according to SEQ ID NO: 61 and the synthetic sequence according to SEQ ID NO: 62. The modified strain herein disclosed may comprise the synthetic sequence according to SEQ ID NO: 60, the synthetic sequence according to SEQ ID NO: 61 and the synthetic sequence according to SEQ ID NO: 62.

SEQ ID NO: 60, 61 and 62 may further comprise an antibiotic marker, as described above. Accordingly, the modified strain herein disclosed may comprise the synthetic sequence according to SEQ ID NO: 63, 64 or 65.

As described above, the live attenuated strain to be modified may be any Salmonella enterica strain. In a preferred embodiment, the live attenuated strain is a Salmonella enterica serovar Typhi strain. Alternatively, the live attenuated strain may be a Salmonella enterica serovar Typhimurium strain. In one embodiment, the live attenuated strain is selected from the group consisting of Ty21a, CVD 908-htrA, CVD 909, Ty800, M01ZH09 (“ZH9”), x9633, x639, x9640, x8444, ZH9PA, DTY88, MD58, WT05, ZH26, SL7838, SL7207, VNP20009, A1-R, or any combinations thereof. In a preferred embodiment, the live attenuated strain is M01ZH09. These live attenuated strains are readily available and would be easily identifiable and commonly used by those in the art. For example, EP 2 801 364 A1 discloses Ty21a, CVD 908-htrA, CVD 909, Ty800, M01ZH09, X9633, X9640, and X8444. In addition, EP 3 917 565 A1 discloses in detail ZH9 strains and derivatives thereof, including ZH9PA. Further references to these strains can be found in the literature, in particular in Petrovska 2004, Hindle 2002, Lehouritis 2017, and Kimura 2010. Also intended to be included are any derivatives or variants of the strains, including genetically engineered or genetically modified strains.

The Salmonella strain herein disclosed may be a genetically engineered non-natural bacterium. For example, the live attenuated strain may comprise an attenuating mutation in a Salmonella Pathogenicity Island 2 (SPI-2) gene and/or an attenuating mutation in a second gene. Suitable genes and details of such a modified strain are as described in WO 2000/68261, which is hereby incorporated by reference in its entirety.

In one embodiment, the SPI-2 gene is an ssa gene. For example, the invention includes an attenuating mutation in one or more of ssaV, ssaJ, ssaU, ssak, ssaL, ssaM, ssaO, ssaP, ssaQ, ssaR, ssaS, ssaT, ssaD, ssaE, ssaG, ssal, ssaC and ssaH. Preferably, the attenuating mutation is in the ssaV or ssal gene. Even more preferably, the attenuating mutation is in the ssaV gene.

The genetically engineered non-natural bacterium may also comprise an attenuating mutation in a second gene, which may or may not be in the SPI-2 region. The mutation may be outside of the SPI-2 region and involved in the biosynthesis of aromatic compound. For examples, the invention includes an attenuating mutation in an aro gene. In a preferred embodiment, the aro gene is aroA or aroC. Even more preferably, the aro gene is aroC.

In yet another embodiment, the genetically engineered non-natural bacterium may be derived from a Salmonella species and may comprise inactivating mutations in one or more genes selected from pltA, pltB, cdtB and ttsA and further comprises attenuating mutations in one or more genes selected from aroA and/or aroC and/or ssaV. Details of said genes and mutations are as described in WO 2019/110819, which is hereby incorporated by reference in its entirety.

It is envisaged that inactivating mutations (e.g., deletions) in the genes pitA, pltB and cdtB will prevent the Salmonella species from producing the typhoid toxin and that inactivating mutations (e.g., deletions) in ttsA will prevent the Salmonella species from secreting the typhoid toxin. It is envisaged that the non-natural bacterium may be derived from Salmonella enterica, in particular.

As would be understood by a person of skill in the art, genes may be mutated by a number of well-known methods in the art, such as homologous recombination with recombinant plasmids targeted to the gene of interest, in which case an engineered gene with homology to the target gene is incorporated into an appropriate nucleic acid vector (such as a plasmid or a bacteriophage), which is transfected into the target cell. The homologous engineered gene is then recombined with the natural gene to either replace or mutate it to achieve the desired inactivating mutation. Such modification may be in the coding part of the gene or any regulatory portions, such as the promoter region. As would be understood by a person of skill in the art, any appropriate genetic modification technique may be used to mutate the genes of interest, such as the CRISPR/Cas system, e.g., CRISPR/Cas 9, TALENS.

Thus, numerous methods and techniques for genetically engineering bacterial strains will be well known to the person skilled in the art. These techniques include those required for introducing heterologous genes into the bacteria either via chromosomal integration or via the introduction of a stable autosomal self-replicating genetic element. Exemplary methods for genetically modifying (also referred to as “transforming” or “engineering”) bacterial cells include bacteriophage infection, transduction, conjugation, lipofection or electroporation. A general discussion on these and other methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); which are hereby incorporated by reference.

The pre-determined genomic location into which the chromosomally integrated synthetic polynucleotide sequence is to be inserted may be a genomic location of low or high transcriptional strength. In some embodiments, the pre-determined genomic location is a genomic location of low transcriptional strength. In some embodiments, the pre-determined genomic location is a genomic location of high transcriptional strength. The skilled person will readily recognise that the location of transcriptional strength will be dependent on the cargo to be delivered. For example, where the cargo to be delivered may be toxic in nature, insertion into an area of the genome having low transcriptional strength, in order to more tightly control its delivery, may be desirable. On the other hand, where the cargo to be delivered requires high levels of expression to be effective and/or has minimal associated adverse effects, insertion into an area of the genome having high transcriptional length may be more desirable.

The present invention enables for any cargo of interest to be integrated into the genome of a Salmonella strain for delivery to a subject in need thereof. Accordingly, the cargo may be a range of different molecules or compounds, dependent on the need of the subject. The cargo may be therapeutic or non-therapeutic in nature. For example, the heterologous polynucleotide sequence may be a DNA or RNA sequence that encodes for an immunogenic compound or a therapeutic compound, for example, a cancer therapeutic. Alternatively, the heterologous polynucleotide sequence may be a DNA or RNA sequence that encodes for proteins associated with the functioning of the bacteria or the ability of the bacteria to act as a delivery vehicle, for example, the heterologous polynucleotide sequence may encode for proteins required for successful transduction. The heterologous polynucleotide sequence may be an RNA molecule intended for delivery to a target cell where it is subsequently translated into the protein via the cell machinery of the host cell, for example, a mammalian cell. The heterologous polynucleotide sequence may encode for proteins associated with the lysis of the bacteria cell in order to release either an RNA molecule or the desired protein. Where the heterologous polynucleotide sequence is an RNA sequence, the RNA molecule may be a mRNA molecule. Where the heterologous polynucleotide sequence is an RNA sequence, the RNA molecule may be siRNA molecule. Where the heterologous polynucleotide sequence is an RNA sequence, the RNA molecule may be a shRNA molecule. Where the heterologous polynucleotide sequence is an RNA sequence, the RNA molecule may be a miRNA molecule.

In a preferred embodiment, the heterologous polynucleotide sequence may encode for an immunogenic compound or a cancer therapeutic. The term “immunogenic compound” refers to any compound which elicits an immune response in a subject in need thereof. For example, the immunogenic compound may be an antigen derived from a bacterial, viral, fungal or parasitic source, which initiates an immune response in the subject against that specific antigen. Alternatively, the immunogenic compound may be a tumour-associated antigen. Tumour-associated antigens are antigenic substances produced in tumour cells, which trigger an immune response in the subject. As such, the present invention may be a cancer vaccine, said cancer vaccine comprising the modified strain herein disclosed, said modified stain comprising a polynucleotide sequence encoding a tumour-associated antigen. The tumour-associated antigen may be selected from the list comprising CD133, CD138, BCMA (B-cell maturation antigen), EGFR (epidermal growth factor receptor), EpCAM (epithelial cell adhesion molecule), GD2, GPC3, HER2, HerinCAR-PD1, MSLN (mesothelin), MG7, MUC1, LMP1, PSMA (prostate-specific membrane antigen, PSCA (prostate stem cell antigen), alphafetoprotein (AFP), carcinoembryonic antigen (CEA), CA-125, epithelial tumour antigen (ETA), tyrosinase, melanoma-associated antigen (MAGE) and p53. Alternatively, the heterologous polynucleotide sequence may encode for a cancer therapeutic. The skilled person will readily recognise that the specific cancer therapeutic to be encoded will be dependent on the specific needs of the subject to be treated. For example, the cancer therapeutic may encode for a therapeutic antibody. The term “therapeutic antibody” as used herein includes whole antibodies and any antigen-binding fragment(s) (i.e., antigen-binding portion) or single chains thereof that result in a therapeutic effect. In one embodiment, the therapeutic antibody may be a monoclonal antibody. The term “monoclonal antibody” as used herein refer to a preparation of antibody molecules of single molecular composition. A monoclonal antibody composition displays a single binding specificity and affinity for a particular epitope. The monoclonal antibody may be a human antibody or a humanised antibody. Alternatively, the cancer therapeutic may be a cytokine or chemokine intended to modulate the immune system of the subject in need thereof.

The use of chromosomally integrated synthetic polynucleotides sequences in the genome of the Salmonella strain allows for the introduction of larger DNA/RNA constructs into the Salmonella chromosome. The heterologous polynucleotide may have a size in the range of 1 Kbp to 10 Kbp. For example, the heterologous polynucleotide may have a size in the range of 1 Kbp to 2 Kbp, the heterologous polynucleotide may have a size in the range of 1 Kbp to 3 Kbp, the heterologous polynucleotide may have a size in the range of 1 Kbp to 4 Kbp, the heterologous polynucleotide may have a size in the range of 1 Kbp to 5 Kbp, the heterologous polynucleotide may have a size in the range of 1 Kbp to 6 Kbp, the heterologous polynucleotide may have a size in the range of 1 Kbp to 7 Kbp, the heterologous polynucleotide may have a size in the range of 1 Kbp to 8 Kbp, the heterologous polynucleotide may have a size in the range of 1 Kbp to 9 Kbp, the heterologous polynucleotide may have a size in the range of 2 Kbp to 3 Kbp, the heterologous polynucleotide may have a size in the range of 2 Kbp to 4 Kbp, the heterologous polynucleotide may have a size in the range of 2 Kbp to 5 Kbp, the heterologous polynucleotide may have a size in the range of 2 Kbp to 6 Kbp, the heterologous polynucleotide may have a size in the range of 2 Kbp to 7 Kbp, the heterologous polynucleotide may have a size in the range of 2 Kbp to 8 Kbp, the heterologous polynucleotide may have a size in the range of 2 Kbp to 9 Kbp, the heterologous polynucleotide may have a size in the range of 2 Kbp to 10 Kbp, 3 Kbp to 4 Kbp, the heterologous polynucleotide may have a size in the range of 3 Kbp to 5 Kbp, the heterologous polynucleotide may have a size in the range of 3 Kbp to 6 Kbp, the heterologous polynucleotide may have a size in the range of 3 Kbp to 7 Kbp, the heterologous polynucleotide may have a size in the range of 3 Kbp to 8 Kbp, the heterologous polynucleotide may have a size in the range of 3 Kbp to 9 Kbp, the heterologous polynucleotide may have a size in the range of 3 Kbp to 10 Kbp, the heterologous polynucleotide may have a size in the range of 4 Kbp to 5 Kbp, the heterologous polynucleotide may have a size in the range of 4 Kbp to 6 Kbp, the heterologous polynucleotide may have a size in the range of 4 Kbp to 7 Kbp, the heterologous polynucleotide may have a size in the range of 4 Kbp to 8 Kbp, the heterologous polynucleotide may have a size in the range of 4 Kbp to 9 Kbp, the heterologous polynucleotide may have a size in the range of 4 Kbp to 10 Kbp, the heterologous polynucleotide may have a size in the range of 5 Kbp to 6 Kbp, the heterologous polynucleotide may have a size in the range of 5 Kbp to 7 Kbp, the heterologous polynucleotide may have a size in the range of 5 Kbp to 8 Kbp, the heterologous polynucleotide may have a size in the range of 5 Kbp to 9 Kbp, the heterologous polynucleotide may have a size in the range of 5 Kbp to 10 Kbp, the heterologous polynucleotide may have a size in the range of 6 Kbp to 7 Kbp, the heterologous polynucleotide may have a size in the range of 6 Kbp to 8 Kbp, the heterologous polynucleotide may have a size in the range of 6 Kbp to 9 Kbp, the heterologous polynucleotide may have a size in the range of 6 Kbp to 10 Kbp, the heterologous polynucleotide may have a size in the range of 7 Kbp to 8 Kbp, the heterologous polynucleotide may have a size in the range of 7 Kbp to 9 Kbp, the heterologous polynucleotide may have a size in the range of 7 Kbp to 10 Kbp, the heterologous polynucleotide may have a size in the range of 8 Kbp to 9 Kbp, the heterologous polynucleotide may have a size in the range of 8 Kbp to 10 Kbp, or the heterologous polynucleotide may have a size in the range of 9 Kbp to 10 Kbp. In a preferred embodiment, the heterologous polynucleotide may have a size in the range of 1 Kbp to 2 Kbp.

In a second aspect, the present invention provides a vaccine composition comprising the modified live attenuated strain herein disclosed. The vaccine composition may be a cancer vaccine. The vaccine composition may be a viral vaccine or bacterial vaccine.

The vaccine composition may further comprise a pharmaceutically acceptable carrier, excipient, or adjuvant. The phrases “pharmaceutically or pharmacologically acceptable” refers to molecular entities and compositions that do not produce an adverse, allergic, or other untoward reaction when administered to an animal, such as, for example, a human, as appropriate. Such preparations will be known to those skilled in the art. Moreover, for animal (e.g., human) administration, it will be understood that preparations should meet sterility, pyrogenicity, general safety and purity standards, as applicable.

As used herein, “pharmaceutically acceptable carrier, excipient or adjuvant” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Examples include, but are not limited to disodium hydrogen phosphate, soya peptone, potassium dihydrogen phosphate, ammonium chloride, sodium chloride, magnesium sulphate, calcium chloride, sucrose, borate buffer, sterile saline solution (0.9% NaCl) and sterile water.

In a third aspect, the present invention provides the modified live attenuated strain herein disclosed for use in the prevention or in the treatment of cancer.

The modified live attenuated strain herein disclosed may be used to prevent, treat or delay recurrence of a neoplastic disease, which is associated with a solid tumour or haematological malignancy. Such diseases include a sarcoma, carcinoma, adenocarcinoma, melanoma, myeloma, blastoma, glioma, lymphoma, or leukaemia. In a preferred embodiment, the neoplastic disease is associated with a solid tumour. In particular aspects, the neoplastic disease is associated with a cancer selected from prostate cancer, liver cancer, renal cancer, lung cancer, breast cancer, colorectal cancer, bladder cancer, pancreatic cancer, brain cancer, liver cancer, lymphoma, leukaemia, gastric cancer, cervical cancer, ovarian cancer, thyroid cancer, melanoma, carcinoma, head and neck cancer, skin cancer or sarcoma.

Neoplasia, tumours, and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. In one embodiment, the neoplastic disease is benign. In another embodiment, the neoplastic disease is malignant. In another embodiment, the neoplastic disease is metastatic. In another embodiment, the neoplastic disease is non-metastatic.

Cancers that may be treated according to the invention include but are not limited to cells or neoplasms of the bladder, blood, bone, bone marrow, brain, breast, colon, esophagus, gastrointestines, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant; carcinoma; undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumour, malignant; thecoma, malignant; granulosa cell tumour, malignant; androblastoma, malignant; Sertoli cell carcinoma; Leydig cell tumour, malignant; lipid cell tumour, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumour; Mullerian mixed tumour; nephroblastoma; hepatoblastoma; carcinosarcoma; mesenchymoma, malignant; Brenner tumour, malignant; phyllodes tumour, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumour of bone; Ewing's sarcoma; odontogenic tumour, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant; neurofibrosarcoma; neurilemmoma, malignant; granular cell tumour, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a preferred embodiment, the cancer may be selected from prostate cancer, liver cancer, renal cancer, lung cancer, breast cancer, colorectal cancer, bladder cancer, pancreatic cancer, brain cancer, hepatocellular carcinoma (liver cancer), lymphoma, leukaemia, gastric cancer, cervical cancer, ovarian cancer, thyroid cancer, melanoma, carcinoma, head and neck cancer, skin cancer or sarcoma. In a further preferred embodiment, the cancer is selected from lung cancer, renal cancer, bladder cancer, ovarian cancer, liver cancer, gastric cancer, colorectal cancer, head and neck cancer or breast cancer.

In a fourth aspect, the present invention provides for the modified live attenuated strain herein disclosed for use in the treatment of infectious disease. The infectious disease may be caused by a virus, a bacterium, a fungus, or a parasite.

In a fifth aspect, the present invention provides for the modified live attenuated strain herein disclosed for use in the treatment of autoimmune diseases or disorders.

Accordingly, the present invention may be used to achieve a therapeutic benefit in a subject in need thereof. A therapeutic benefit or beneficial effect is any objective or subjective, transient, temporary, or long-term improvement in the condition or pathology, or a reduction in onset, severity, duration, or frequency of an adverse symptom associated with or caused by cell proliferation or a cellular hyperproliferative disorder such as a neoplasia, tumour or cancer, or metastasis, or by an infectious disease, or by an autoimmune disease/disorder. It may lead to improved survival. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumour or cancer, or metastasis, or characteristics of an infectious disease, or of an autoimmune disease/disorder. A therapeutic benefit or improvement in the context of cancer therefore may be, but is not limited to, destruction of target proliferating cells (e.g., neoplasia, tumour or cancer, or metastasis) or ablation of one or more, most or all pathologies, adverse symptoms or complications associated with or caused by cell proliferation or the cellular hyperproliferative disorder such as a neoplasia, tumour or cancer, or metastasis. However, a therapeutic benefit or improvement need not be a cure or complete destruction of all target proliferating cells (e.g., neoplasia, tumour or cancer, or metastasis) or ablation of all pathologies, adverse symptoms or complications associated with or caused by cell proliferation or the cellular hyperproliferative disorder such as a neoplasia, tumour or cancer, or metastasis. For example, partial destruction of a tumour or cancer cell mass, or a stabilization of the tumour or cancer mass, size or cell numbers by inhibiting progression or worsening of the tumour or cancer, can reduce mortality and prolong lifespan even if only for a few days, weeks or months, even though a portion or the bulk of the tumour or cancer mass, size or cells remain. A therapeutic benefit or improvement in the context of infectious disease may be, but is not limited to, a reduced viral, bacterial, fungal or parasitic load. A therapeutic benefit or improvement in the context of an autoimmune disease/disorder may be, but is not limited to, a reduction in inflammatory markers or a reduction in autoantibody titres.

The live attenuated Gram-negative bacteria may be administered orally. As used herein, the terms “oral” or “orally administered” are used interchangeably and refer to the Salmonella bacteria being administered via the mouth of the subject in need thereof. However, it is also contemplated that other methods of administration may be used in some cases. Therefore, in certain instances the modified live attenuated Salmonella bacterium of the present invention may be administered by injection, infusion, continuous infusion, intravenously, intradermally, intraarterially, intralesionally, intravaginally, intrarectally, intramuscularly, intraperitoneally, subcutaneously, subconjunctival, mucosally, intrapericardially, intraumbilically, intraocularally, intracranially, intraarticularly, intraprostaticaly, intrapleurally, intratracheally, intranasally, via a catheter, via a lavage, or by other method or any combination of the forgoing as would be known to one of ordinary skill in the art (see, for example, Remington's Pharmaceutical Sciences, 18th Ed. Mack Printing Company, 1990). Alternatively, in the context of the treatment of neoplastic disease, preferable routes of administration are intratumorally and peritumourally.

In one embodiment, the modified strain herein disclosed may be administered in combination with another therapeutic agent, said therapeutic agent being dependent on the disease to be treated. For example, the modified strain may be administered in combination with an immunotherapy, a chemotherapy, a radiotherapy, an anti-viral therapy, an antibacterial therapy, an antifungal therapy or an antiparasitic therapy. Chemotherapy agents include, but are not limited to, alkylating agents, plant alkaloids, antitumour antibiotics, antimetabolites and/or topoisomerase inhibitors, or any combination thereof.

In a preferred embodiment, the immunotherapy is a checkpoint inhibitor, an antigen specific T-cell, a therapeutic antibody, or a cancer vaccine.

The immunotherapy may be a checkpoint inhibitor, the term “checkpoint inhibitor” herein refers to a blocking agent directed against a checkpoint molecule. The blocking agent may be an antagonist, an inhibitor, or a blocking antibody. Accordingly, the blocking agent may be a small molecule or a biologic drug, in particular instances it is a monoclonal antibody. In a preferred embodiment the checkpoint inhibitor is directed against CTLA-4, PD-1, PD-L1, LAG-3, TIM-3, BTLA, TIGIT, VISTA or any combinations thereof.

The checkpoint inhibitor may be a therapeutic antibody directed at the specific cancer or tumour of the subject in need thereof. In particular embodiments, the therapeutic antibody may be a monoclonal antibody, and even more preferred, a humanised or human monoclonal antibody. Methods of obtaining such monoclonal antibodies are known to those skilled in the art. The therapeutic antibody may block an abnormal protein in a cancer cell, attach to specific proteins on cancer cells or be conjugated to a cytotoxic molecules, such as an anticancer drug. The latter flags the cancer cells to the immune system so that the abnormal cells can subsequently be targeted and destroyed by cellular components of the immune system. Examples of monoclonal antibodies that are checkpoint inhibitors include, but are not limited to, ipilimumab (Yervoy®), nivolumab (Opdivo®) and pembrolizumab (Keytruda®).

In another embodiment, the immunotherapy may be an adoptive cell therapy, wherein immune cells are transferred into a patient/subject, most commonly due to their improved functionality and characteristics. The cells to be transferred may have originated from the subject (autologous) or from another subject (allogeneic). Examples of such adoptive cell therapies include, but are not limited to, engineered or non-engineered macrophages, engineered or non-engineered yδ T cells, engineered or non-engineered natural killer cells. Accordingly, adoptive cell therapies include, but are not limited to, tumour-Infiltrating lymphocyte (TIL) therapy, Engineered T Cell Receptor (TCR) therapy and/or natural killer (NK) cell therapy, the details of which will be well known to those skilled in the art (Adoptive cellular therapies: the current landscape, Rohaan et al. 2019, Virchows Arch. 474(4): 449-461).

The immunotherapy may be a CAR T-cell therapy. The CAR T-cell therapy may be allogeneic or autologous. In some instances, the CAR T-cell therapy may be directed against the antigen CD19, which is present in B-cell derived cancers. Accordingly, such therapy may be particularly suited for B-cell derived cancers, such as acute lymphoblastic leukemia (ALL) and diffuse large B-cell lymphoma (DLBCL). In other instances, the CAR T-cell therapy will be directed against tumour-associated antigens (TAAs) and are accordingly more suited for the treatment of solid tumours. Examples of such antigens include, but are not limited to, CD133, CD138, BCMA, CEA, EGFR, EpCAM, GD2, GPC3, HER2, HerinCAR-PD1, MSLN, MG7, MUC1, LMP1, PSMA and PSCA. Such techniques will be known to those skilled in the art and the reader is directed to the review entitled “Adoptive cellular therapies: the current landscape” for further information (Rohaan et al. 2019, Virchows Arch. 474(4): 449-461).

The additional therapeutic agents described above may be administered before the modified Salmonella strain herein disclosed. The additional therapeutic agents described above may be administered after the modified Salmonella strain herein disclosed. The additional therapeutic agents above may be administered at substantially the same time as the modified Salmonella strain herein disclosed. Where the intention is to administer the modified Salmonella strain herein disclosed for the purpose of activating the subject's immune system, it is preferable for the strain to be administered prior to the administration of the additional therapeutic agent. Accordingly, the modified Salmonella strain may act as a priming agent. The modified Salmonella strain and the additional therapeutic agent may act in an additive or synergistic manner to improve the clinical outcomes of the subject in need thereof.

In a sixth aspect, the present invention provides for a method of treating, inhibiting or controlling a neoplastic disease, or an infectious disease, in a subject, wherein the method comprises administering to the subject a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

in one embodiment, the chromosomally integrated synthetic polynucleotide sequence is located within at least one genomic locus defined by any one of SEQ ID NOs: 1-30, or a sequence comprising at least 75% identity to any one of SEQ ID NOs: 1-30; at least 80% identity to any one of SEQ ID NOs: 1-30; at least 85% identity to any one of SEQ ID NOs: 1-30; at least 90% identity to any one of SEQ ID NOs: 1-30; at least 91% identity to any one of SEQ ID NOs: 1-30; at least 92% identity to any one of SEQ ID NOs: 1-30; at least 93% identity to any one of SEQ ID NOs: 1-30; at least 94% identity to any one of SEQ ID NOs: 1-30; at least 95% identity to any one of SEQ ID NOs: 1-30; at least 96% identity to any one of SEQ ID NOs: 1-30; at least 97% identity to any one of SEQ ID NOs: 1-30; at least 98% identity to any one of SEQ ID NOs: 1-30; or at least 99% identity to any one of SEQ ID NOs: 1-30.

In a seventh aspect, the present invention provides for a method of modifying a live attenuated strain of Salmonella, said method comprising inserting a synthetic polynucleotide sequence into a pre-determined pseudogenomic location of the live attenuated strain of Salmonella, said sequence comprising at least one defined recombination site for the introduction of a heterologous polynucleotide sequence encoding a polypeptide, wherein said chromosomally integrated synthetic polynucleotide sequence is located within at least one genomic locus according to defined by any one of SEQ ID NOs: 1-30, or a sequence comprising at least 70% identity to any one of SEQ ID NOs: 1-30 (Table 1).

in one embodiment, the chromosomally integrated synthetic polynucleotide sequence is located within at least one genomic locus defined by any one of SEQ ID NOs: 1-30, or a sequence comprising at least 75% identity to any one of SEQ ID NOs: 1-30; at least 80% identity to any one of SEQ ID NOs: 1-30; at least 85% identity to any one of SEQ ID NOs: 1-30; at least 90% identity to any one of SEQ ID NOs: 1-30; at least 91% identity to any one of SEQ ID NOs: 1-30; at least 92% identity to any one of SEQ ID NOs: 1-30; at least 93% identity to any one of SEQ ID NOs: 1-30; at least 94% identity to any one of SEQ ID NOs: 1-30; at least 95% identity to any one of SEQ ID NOs: 1-30; at least 96% identity to any one of SEQ ID NOs: 1-30; at least 97% identity to any one of SEQ ID NOs: 1-30; at least 98% identity to any one of SEQ ID NOs: 1-30; or at least 99% identity to any one of SEQ ID NOs: 1-30.

The defined recombination site will be present in the synthetic polynucleotide sequence. Appropriate recombination sites will be present to allow the introduction of any cargo into the landing pad once integrated into the chromosome. The landing pads of the present invention may comprise a specific attB recombination site that has been previously introduced in a pseudo-genomic position in the genome. The payload may be surrounded by attP recombination sites that react uniquely to a particular attB sequence from the genome. If there are two or more attB sequences in the genome, the payload will only be inserted into the matching attB sequence.

The inventors of the present invention have surprisingly found that by introducing at least one chromosomally integrated synthetic polynucleotide inserted into a pre-determined pseudogenomic location of a Salmonella strain, any desired cargo of interest can be efficiently and easily integrated into the chromosome of the Salmonella strain at regions where gene expression has previously been characterized (enabling control over transcriptional strength). Furthermore, the inventors have shown that modifying the Salmonella strain in this way does not affect strain growth, viability, or its potential downstream therapeutic effects.

The invention is further described with reference to the following non-limiting examples:

### EXAMPLES

### Example 1: Validation of ZH9 Strain Genome and Computational Selection of Landing Pad Locations

The ZH9 Salmonella strain, obtained from Glycerol Master Stock, was grown for 8 consecutive days in vLBaro media, with subculturing every 12 h, at 37 °C and 200 rpm. Aliquots were taken at days 1, 4, and 8, and the genome subsequently sequenced. The assay allowed the establishment of a working protocol to assess the genome sequence of the ZH9 strain, with or without the presence of the landing pad sequence.

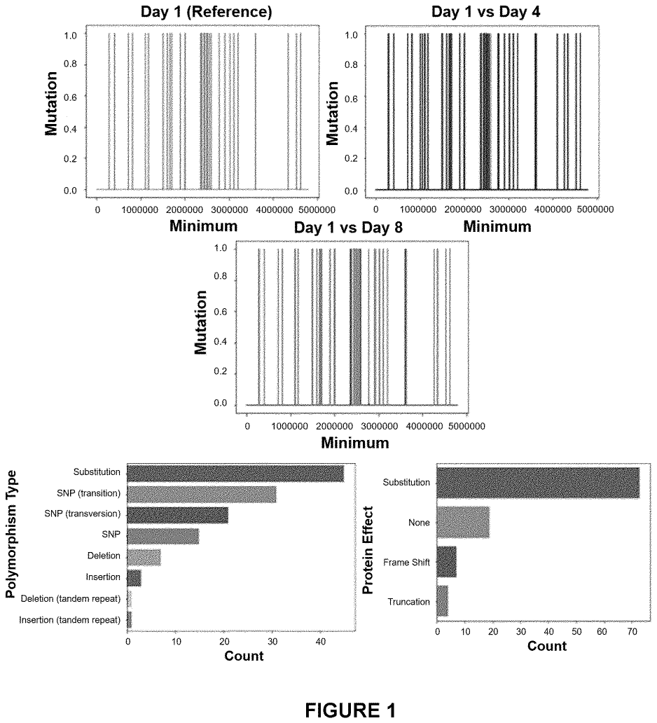

The sequence for the genome of ZH9 was aligned to a reference genome (the Ty21a Salmonella strain) and the differences analysed (see FIG. 1). Major differences were found, as expected in the genes aroC and ssaV. Other small variations were identified, although these did not affect known/characterised genetic elements.

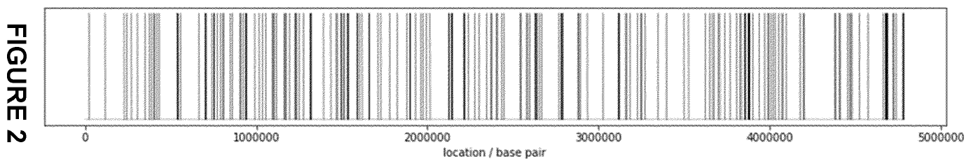

Following the establishment of a reference genome, the location of 218 pseudogenomes in the chromosome were identified (see FIG. 2). Specific pseudogenomes were subsequently identified that had known transcriptional strength and therefore particularly useful for the insertion of heterologous cargo.

### Example 2: Design of Landing Pad Sequences and Integration into Salmonella Chromosome

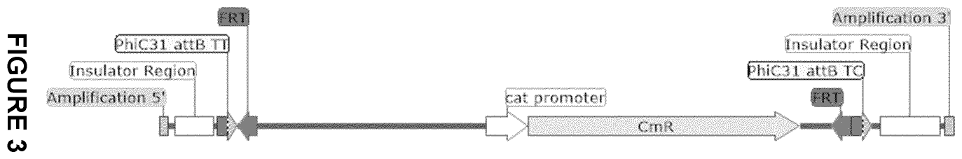

The landing pad DNA sequence comprised an antibiotic marker (CmR), to select clones with successful integration; two FRT sites, to remove the marker upon clonal confirmation; two PhiC31 attB sites (containing unique TT and TC cleavage sites), used to integrate any cargo into the landing pad once integrated into the chromosome; and two insulator regions containing strong terminators, which avoid transcriptional readthrough from the genome to impact on the integrated cargo. Addition of recombination overhangs, specific per each location of interest was added using two standardised amplification sites (5′ and 3′) (see FIG. 3).

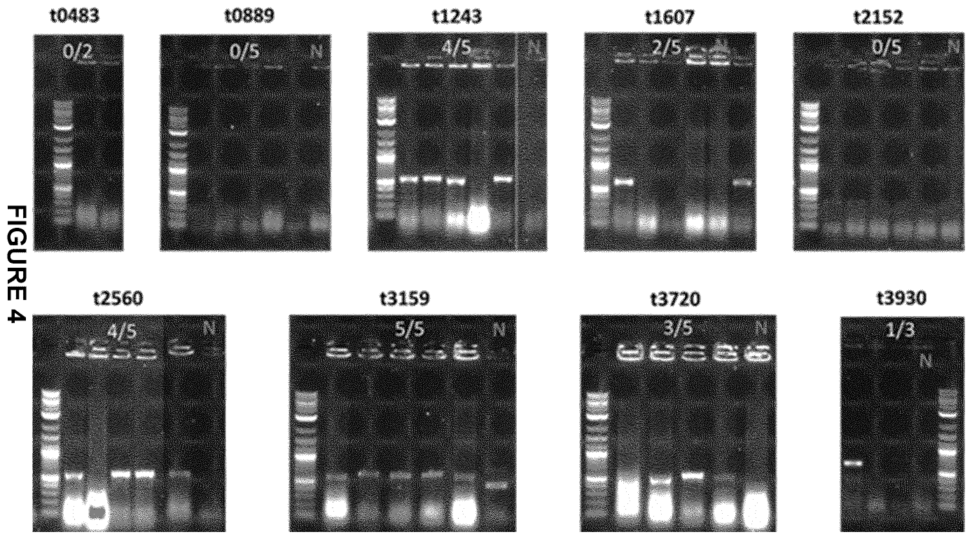

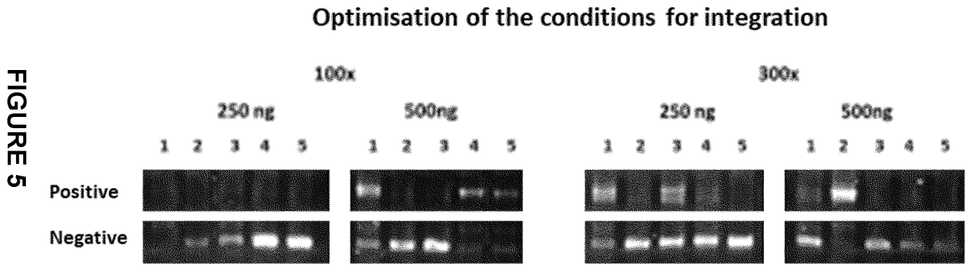

A small-scale trial was performed using landing pad amplicon with overhangs for locations t0483, t0889, t1243, t1607, t2152, t2560, t3159, t3720, and t3930. Integration was validated by a PCR reaction designed to yield amplicon (~500 bp) only in case of successful recombination (see FIG. 4). Optimisation of the conditions for integration using landing pad amplicon with overhangs for location t2560. To assess the optimal integration conditions, cells were concentrated either a 100× or 300× from OD₆₀₀~0.6 during electrocompetent cells preparation and electroporated either with 250 ng or 500 ng of DNA. Successful integration was assessed using primers that anneal outside the chromosomal location. Best conditions were found to be using 500 ng of DNA in 100× cells (see FIG. 5).

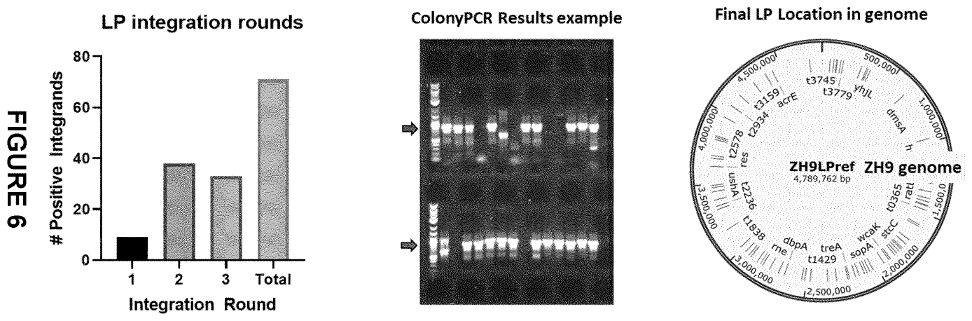

Seventy-two different ZH9 mutant strains, each containing a single landing pad sequence in a pseudo-gene location, as validated by colonyPCR, were generated. The different landing pad sequences were interspaced across the ZH9 chromosome (see FIG. 6).

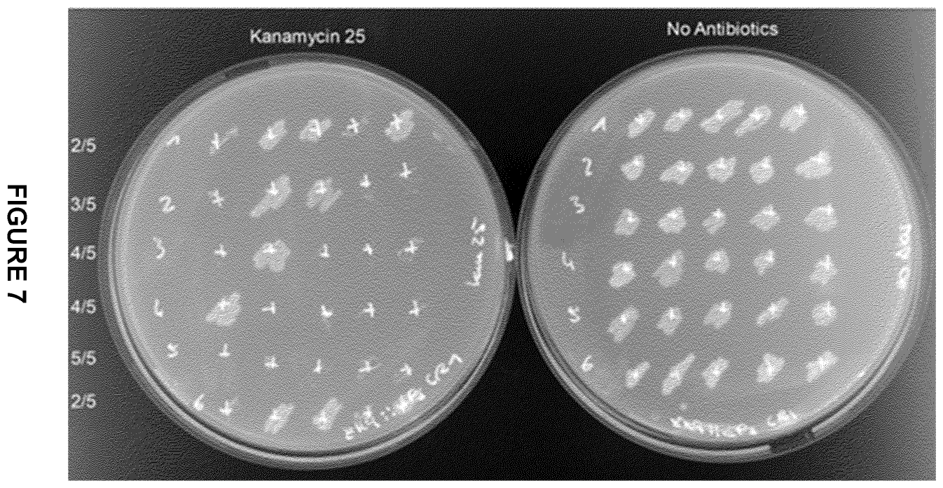

In order to eliminate the auxiliary plasmid after recombination, cells were grown at 42 °C for 16 h and then replica plated in antibiotic (Kanamycin) or non-selective media. Cells where the auxiliary plasmid were successfully removed were shown to grow only in the “No Antibiotics” plate (see FIG. 7).

### Example 3: Landing Pad Effect on Salmonella Growth

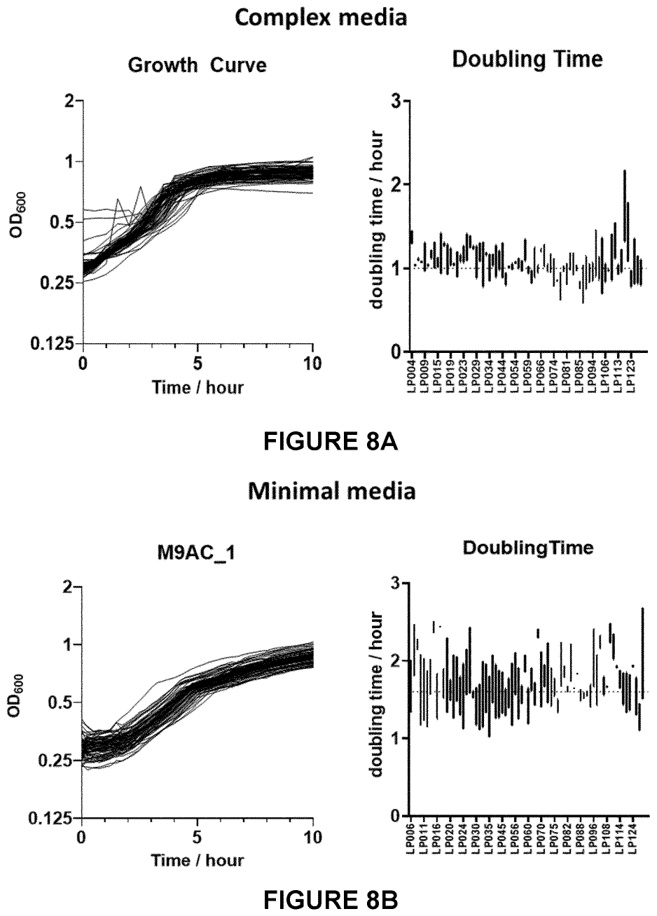

Samples were grown in either complex media (vegan Lysogeny Broth) or minimal media (M9 media) for 12 h at 37 °C. No significant growth differences were observed in the growth curves of all the strains in either rich or minimal media. The rate of growth and the maximum cell density was comparable between all landing pad-containing strains (see FIG. 8A and FIG. 8B).

### Example 4: Creation of a Shuttle Vector to Introduce Cargo into the Chromosomally Integrated Landing Pad

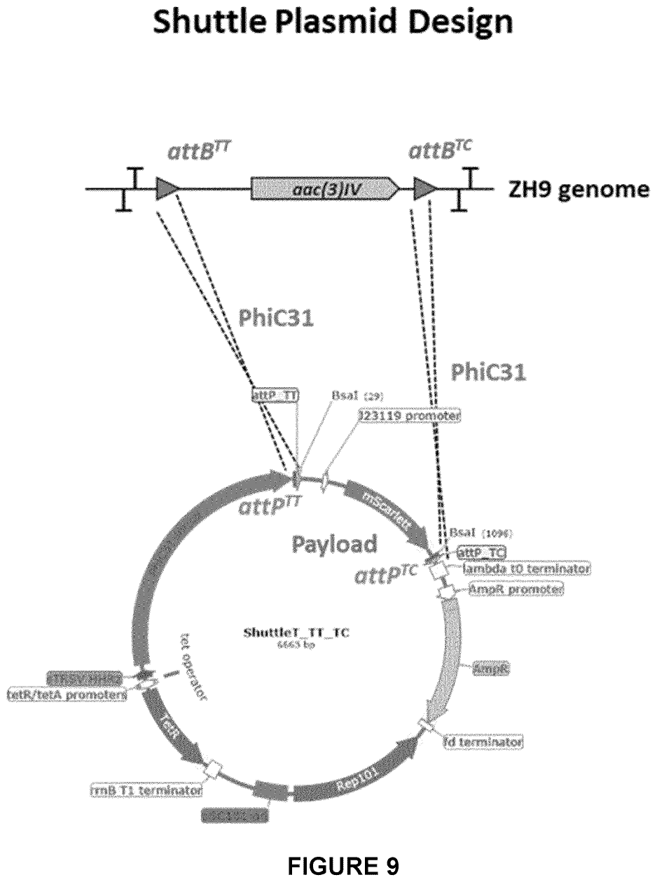

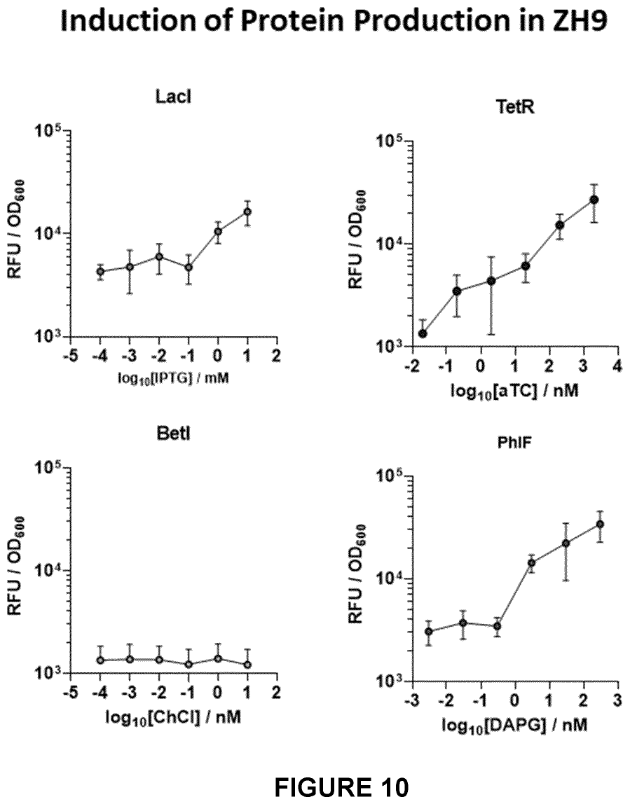

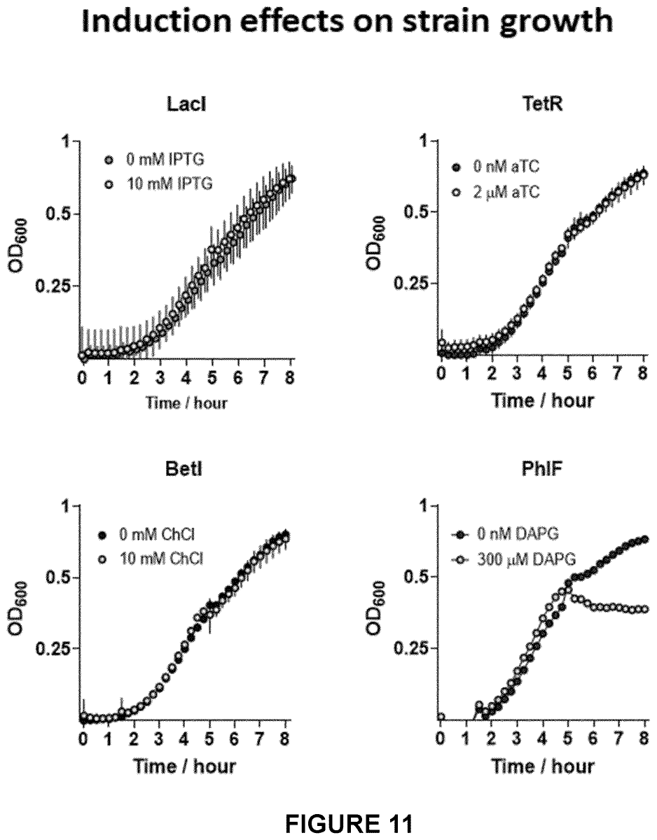

A Shuttle vector, containing a serine recombinase (e.g., PhiC31) was used to introduce cargo into the landing pads in a targeted manner (see FIG. 9). Once the cargo was introduced, the vector was removed from the therapeutic strain. Expression of PhiC31 must be tightly controlled to prevent leaky expression of the recombinase and robust to allow for reproducible, high-throughput loading into strains. To this end, four different expression systems were tested: Lacl, TetR, Betl, and PhIF. The best expression system was found to be TetR, which offered tight repression of expression whilst minimizing effects on strain growth upon induction (see FIGS. 10 and 11).

### Example 5: Expression of Cargo as a Function of Genomic Position

Expression of cargo as a function of its genomic position was assessed in a total of 70 different ZH9 strains, each containing one landing pad, with two independent assays.

The first assay was performed using chloramphenicol acetyl-transferase (CAT), an antibiotic resistance gene against chloramphenicol. Landing pad-containing strains were challenged against the antibiotic and the OD₆₀₀ measured during exponential growth. Samples with higher expression contain more copies of CAT and thus grow faster.

The second assay was performed using mScarlet, a bright red fluorescent protein. Landing pad-containing strains were grown in rich media and the fluorescence output measured at different growth stages (OD₆₀₀~0.5 shown here). Samples with higher expression have higher fluorescence levels.

For the characterisation of landing pads using mScarlet, samples were electroporated with 5 nM pShutTeR. Positive clones (as determined by coloPCR) were re-streaked 4× in in vLB devoid of antibiotics (2× single clones per round). Plasmid curing was determined by replica plating in media supplemented with Kanamycin (or without, 12× clones each strain).

Final clones were isolated and stored as glycerol stocks. Cells were then grown overnight in vLBaro and sub-cultured in triplicate. Growth measurements were monitored every 15 min and mScarlet fluorescence at the following thresholds: OD₆₀₀~0.3, 0.5, 1.0. Fluorescence was normalised per cell unit and samples compared (normalisation done using GraphPad built-in algorithm, where samples are normalised as (x−xmin)/xmax*100).

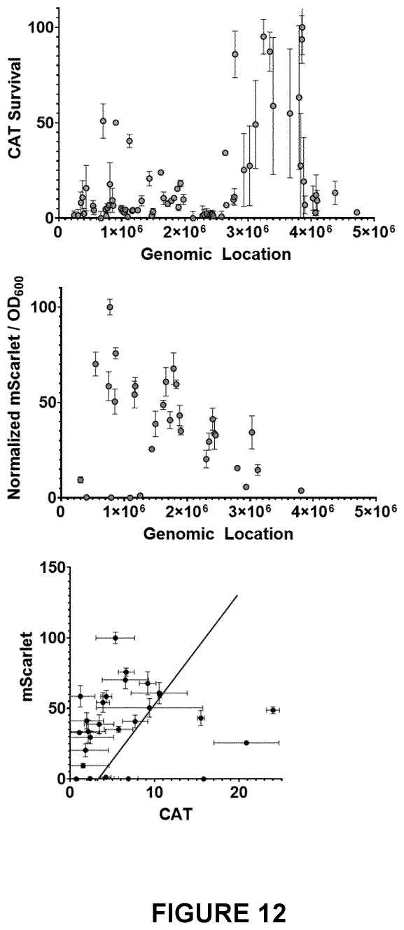

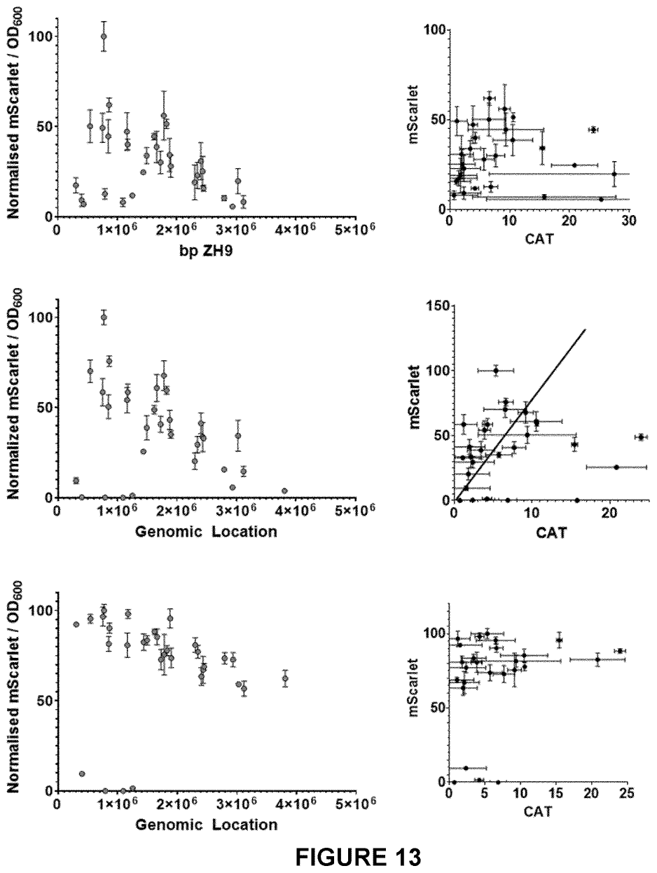

A total of 70 different ZH9 strains, each containing one landing pad, were validated using the two independent assays described above (see FIGS. 12 and 13).

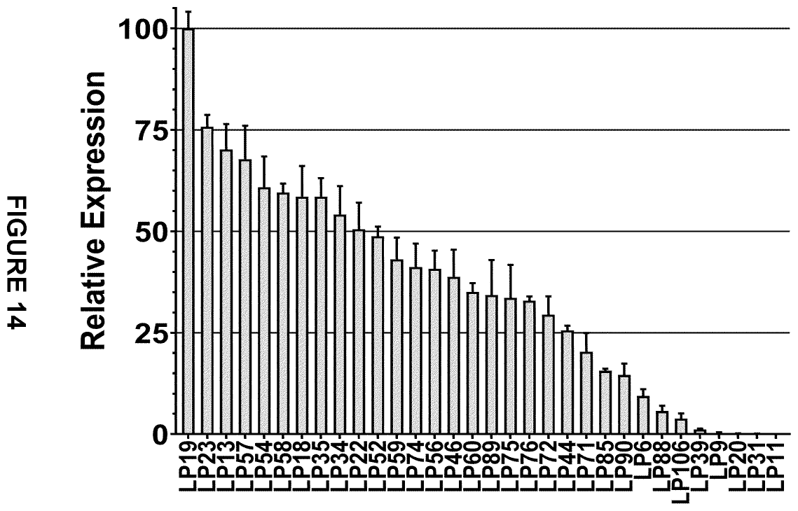

The above experiments allowed ranking of several of the landing pad strains by their relative transcriptional strength (see FIG. 14), where samples LP19 (t0687) and LP23 (t0771) are considered best candidates for integration of cargo with high relative expression levels.

### Example 6: Creation of Multi-Landing Pad Strains

S. enterica ZH9 strains containing one landing pad, two lading pads, or three landing pads were constructed using sequential integration rounds via recombineering.

Initially, ZH9 strain containing a landing pad LP1 (SEQ ID NO: 63) into the t0687 locus (SEQ ID NO: 5) was constructed. The antibiotic resistance cassette was removed using FLP. Afterwards, the auxiliary pCP20 plasmid expressing FLP was cured from the resulting strain (Strain A).

Strain A was used as template to generate strains with two landing pads by integrating landing pad with LP2 (SEQ ID NO: 64) into the t0771 locus (Strain B1) or LP3 (SEQ ID NO: 65) into the t1716 locus (Strain B2). As previously done, the antibiotic resistance marker was removed using FLP and the final strains were cured from all auxiliary plasmids. Finally, a single strain containing three landing pads was generated by using either strain B1 or B2 as a template and integrating the remaining landing pad into the corresponding position (e.g., integrating LP2 into the locus t0771 when using strain B2 as the template). The final strain was removed from antibiotic resistance marker and auxiliary plasmids (Strain C).

All four strains were confirmed by genome sequencing.

### Example 7: Assessing Genome Stability of Strains Containing Up to Three Landing Pads

S. enterica ZH9 strains containing one landing pad, two lading pads, or three landing pads were grown in 10 mL vLB media overnight from glycerol stocks. Samples were then diluted 1:100 in fresh 10 mL of vLB media and growth was restarted. The procedure was repeated to up to four days.

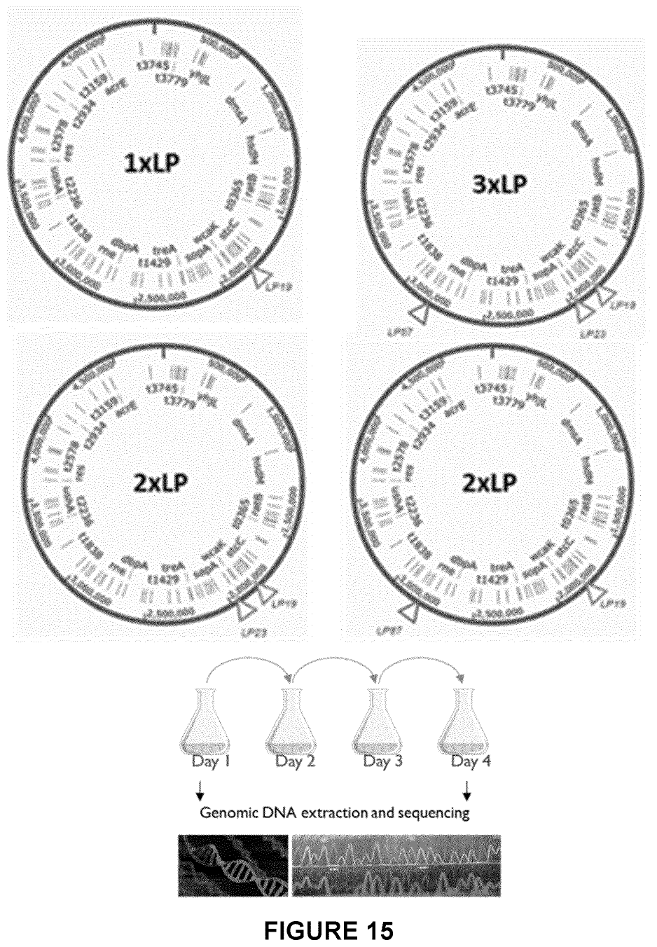

Both on day 1 and day 4, an aliquot was taken, and the genomic DNA of each sample was extracted and sequenced. Samples were compared between both days and landing pad locations were screened for mutations or rearrangements. No instability was observed in any of the landing pads and in any of the strains after four days of continuous growth in rich media (FIG. 15).

### Example 8: Loading of a Cargo in a Strain Containing Three Landing Pads

A DNA sequence encoding for mScarlet (red fluorescent protein) was loaded into a shuttle vector after introducing the corresponding attachment sites attP allowing the loading of the cargo LP1 position of Strain C. This shuttle vector contains a l-Scel cleavage site, a chloramphenicol resistance marker, and a pSC101 origin of replication.

An auxiliary plasmid containing PhiC31 circuit as described in example 4 was introduced into Strain C. This plasmid contains a carbenicillin resistance gene and a p15A origin of replication. Strain C with the auxiliary plasmid was then transformed with the shuttle vector containing mScarlet in TT/TC attachment sites (locus t0687).

The auxiliary plasmid containing the serine recombinase was cured spontaneously after overnight subculturing at 37 °C in vLB media. The Shuttle vector, however, was only cured after transformation of Strain C-mScarlet with a I-Scel expression plasmid that cleaved the Shuttle vector and promoted it's curing. Intriguingly, while two variants of the I-Scel expression plasmid were tested, curing was more efficient when lambda repressor was placed upstream i-scei. This auxiliary plasmid was then spontaneously cured by subculturing at 37 °C overnight.

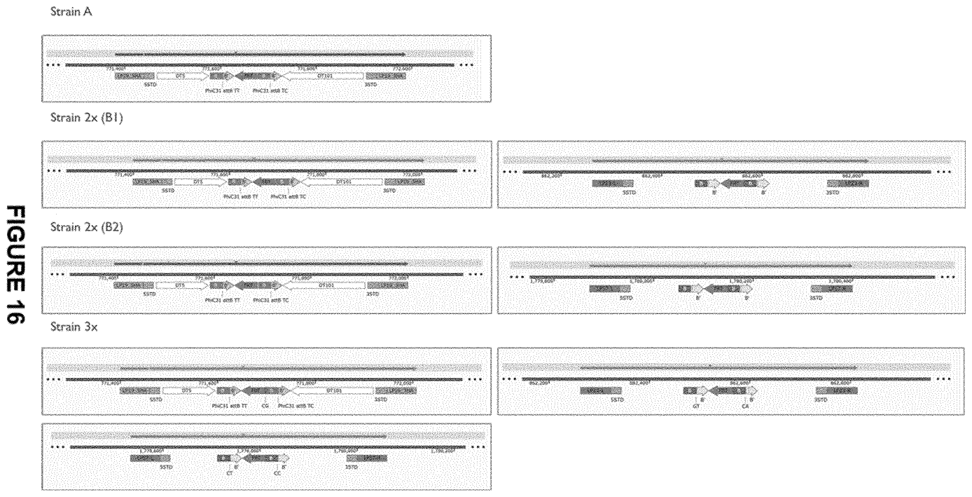

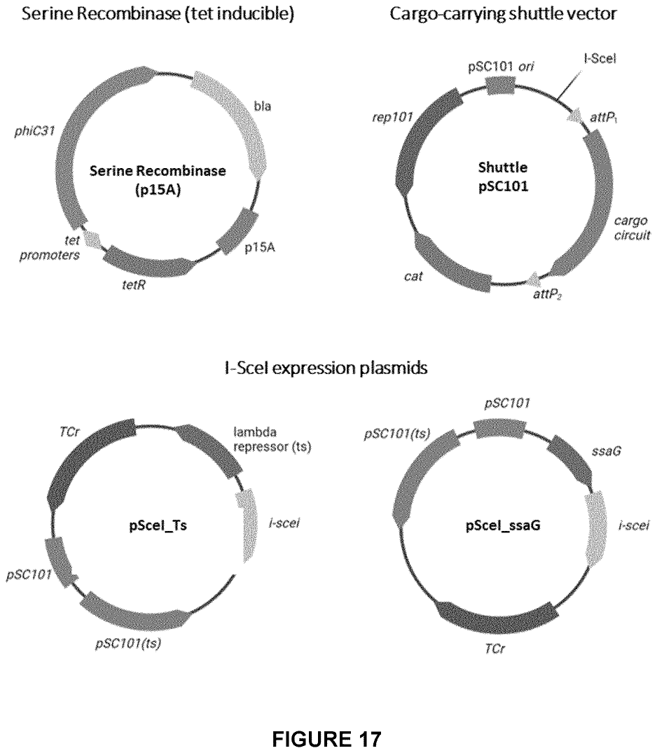

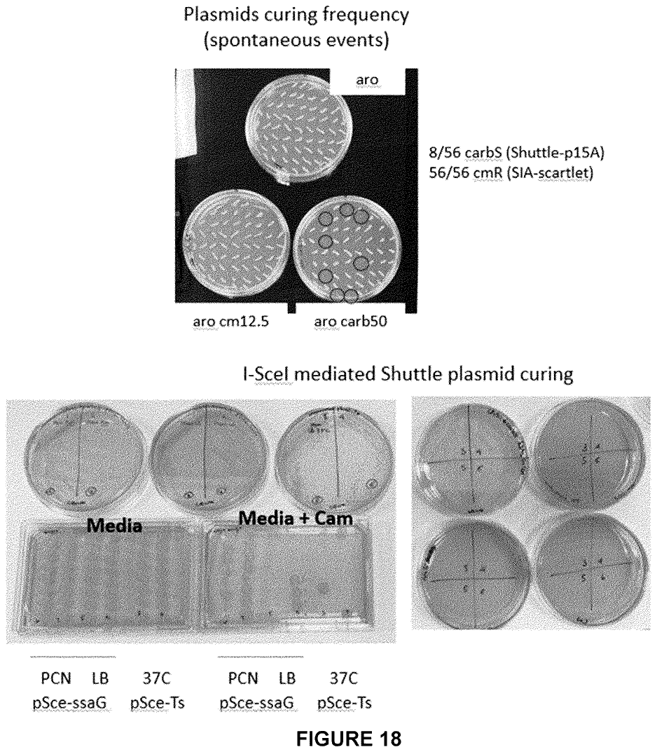

Final strain (Strain C-mScarlet) was unable to grow in any antibiotics and contained mScarlet integrated in position LP1 of Strain C (FIG. 16).

SEQUENCES FORMING PART OF THE DESCRIPTION
SEQ ID NO: 1:
TTGAAACTGAAAACGAATACCCATCTTTTATCCGGGCTACAGGCATGTCTGTTTTGTGT
TACCGCTTTTTTTGCCCAGGCGGCGCAAAGCAGCGCCCCATGGCAGGAGCCTGACGTCT
CAACTGCCGCCATTAACGGCACGCCGCCGCTGGCTGACGGCGTGACGATCCCGGTTTAT
CAGGGCAGCGTTCAGTTGCGATCTGATGCAGCGAATCCCGTCGATTATTCCGCGAAACC
CAGCCAGTTCAGCACCAGTGATGTCGGCAGCGCGCTGACCTTAACCAATCCGCGCGATG
CCGAAGGCGATATTTTCGCCGAGCCGCCGCTGGTCTGGCAGAGTGAACGGATACCCTCC
GTCACGCTGGTATGGGCCGACGCCGCCACGCCGGAGACGCCGCTGTCTCCACAATCGTC
GGCAAACCTGACGTTCTGTGAGCAAAATATGGCAGGCCGCCATCTGGTAGTCTGGTCGC
AACTGGATACCTCAACCGCTATGCCGCCGCTGTGGTTGCTTACCCGCACCGGCGTGCCT
TATAACACGGCGGTCGAAGTGCTTGAACAAAAGTTTGCCGTGGATATCGCGCCCGCCGT
GGGTGACCCGGTAACCCTTACCGCCGATCATCTGGATGAGTCGCTTAACGCCGCGAAGG
TGAAAGCGGGCGAAAGCATCACGCTGACGGTGAATACCAAAGGTTGTAACGGTGAGCCA
GCGGGCAATATCGCGTTTGTGATTACCCGCGGCGATGCGCAAAACCGCCAGGGCGTGGT
GAACAATACCGCGCCGGTACGCGTCGGCAACACCGAACTCACCACCACTGCCACCGAAT
ACCACGGCACGACCAACGCGGAAGGGGTGGCGACGGTGACCGTCACCCAGGCTAACGGG
CCGGGCGTTAAAACCCCGCTGATGGCGCACCCGTCGAACGCGCCTGCGCTGAAAGCCAG
CGCGGATGTGATTTTTACCACCCTCACCAGCCCGGACAGCAGCAGCGCCAACATGTACG
GCCATATGGCGGACAGTTCCACCGCCACGGTGGACGGCGCAAGCTACACTTTTGACCGC
CCCAAACTGGCGGCGGAAACCGAGGGCGAAGACCGCGTCGCCAGTATCAATAATGAAAA
CTGGGCGCAGTTCACCTGGGGCCATGCGGATAAACACTGCGATATTCTGCCGGATGCGC
GCCAACTGGAAGGGTTAAAAATCGAGCGCGGCGATTTAGCCACCACTCTCGGCTGGCCG
GTGGGGTGACATCCGGTGACGAAGAGCACTGGTCCTCCTCACAGGGGGCGAGCGCAACT
GACCACATCAGCATTGATATGCGCAGCCGCGCCCTTACGCAGATGCCGGACGCCACGCA
ATCGCTGGTGAGCTGTGTGGATAAAGCCTCGCCTGCGGTCACGCCGAAATTAGTGATTA
GCACCGATAATTTTGATTCGACGGCCAACGCGGCGAAAGTGAAGGTGGGCGAAGAGATC
AACATGAAGATCGCCGTCACCGACAGCGCCACCAATAAACCGCTGCCTTATCGCTACTT
TAATGTCTACCTGGGCGACGAGCAAAATCGCCAGAATCAGAAGAACGCCGATCTTGATG
CCGCGCATCAGTGGACGGATGAACCGGTAGTTATCGCCAATCTGGACGGCGGCGACGGT
CACTATCACGGCGTCACCGACGCCAACGGCCAGTTTTCGCTGGCGCTGACCCAGGACAA
GGGCGCTGGCGTATTAACCCCGGTGCGCGTGGTGCTGTTCGACGGCACGGAAGCGACGC
AGAATGTGATTTTTACCGTCGTCACCAGCCCGGACGTGACGCAGGCCCGTATGTGGGGC
CATATGCAGGGCGTGGTGGAAGCGGGCAATATTTACAAACGTCCGCTGCTGGCGGAAGA
GGCCGCGCAGGATACCGGTTCCGAATTTGAGAATAACGAGTACTGGGCCACGTTTAACT
CAGTGACGGCAGCGACAAACCAGTGCGGCGCAGGCCAGGTGCCGGGACAACTGCTGCTG
GATACGCTGTATGAAGCCCATTCCGGCAATACGATGGAAACGACTTACGGCTGGCCGAC
ACAGAAACATAGCTATATTGCGGCGGATACCGATGGTTCCACCACTGCGCACGTTAACC
TGGCGACCGGCGCGGACAGCCTGTTCAGCGGCGCTGAACCGAACTATCTCTCCTGCTCC
GGTAACGAGTTGGTGACCAGCCTGGACGTGTACTTTGATGGCAACGAGTCGTTACGTAA
TGCGGTAGCCAAAGTGGGCGAAAAAATTACGATGAACGTCCATTCCGTCAACGCGCTGA
ACGGGTTAAGCGTGCCCAACGCCAGCTTTACCGTCACCATGTCGCACGGTAAAAATCGC
GCCAACGCCACCACGGGCTTTACCGATCCCAGCGACGGAACGCTGGTGATGGGCGGAAC
GTCTTTCGGCTCTTCCCTGGCGTCGATGACGTACCAGGGCATGACCGATGCCGCAGGCA
ACGCCACGCTGGTCATTGAACAACCGCAGGGTGTCGGCTTGCTGACGCCGCTGACCGTA
CTGCCGGTTAACTCGCTGATCACCACCCCCGTTAACCGCAGCGTGAAGTTTACCGTTCC
CACCAGCCCCGATACGCCGGATGCGCAAATGTGGGGCCATATGTCCGACGCCATCACGG
TGGGCGATATGACCTTTGAGCGACCGAAACTGGCGGCGGAGGTTGCCGCGACCCGCACC
CAAACCGAGGCCAACGAGAGCTGGGCGCGCGCTACCCATGCCGATGCGGTAGGCAATAC
GGCGGCGGGCGGCTGCGCCGCTAACCGATTGCCGCGCGCCGATCAACTGGAGGCGCTTT
ACGCCGCTAACCGCGACGGGGCTATCAATAGCACGCACGGCTGGCCGGTGCTGATTAAT
TACTGGACCTCCACCTGGCAAAGCGCCACAACGTGGAAACTGATAGCGCTTTCTAACGG
TAGCGAATTTCCGGGCGGCGCTGGCGCATCCGATTACGTCAGTTGCCTTGCCAGCGATA
ACCCGACGGCAGCCTCCATCACCATTGAGCCGGTGAATACGTCATTGTGGTATGACGAG
AACAGCGAACACGCGGTGAAGGTGAAAAAGGGAGATACGCTCCAGCTTAAAGTGACGGT
AAAAGACGCCAGCGGCAATCCGCTGCCGCAAGCGCCGTTCGTGCTCAGCCGCGGCGACG
GCATCGTCTCGGCGGTAGTGATTGACGGCGATTCGCTCAACGACACCGCCACCAAAATC
GGCGGCATGACCGGTGAAAACGGCAGCAAAATCATCAACGTCACCCGCCCGGATGCCCA
CGGGACGAAAGTCGCGATTACGGCGGCGCTGTACGATAACGCCAGCGCGACGGCCAGCA
TCGACACTATTTTTACTGTGGTCACCAGCCCTAATAGCGACAAAGCCAAAATGTGGGGC
CATATGCCGGAAACGACCACGGCGGCCAACGGCGTGGTGTTTAAGCGCCCGCTGTTATC
GGCTGAAATCGCCAGCGGCTTTACCCACGGCGACAATACGGAAAACAACGAAGCCTGGG
GAATCGTCGATTTTGAAGTGGCGAACGACGCCTGTGGCGCGGGATACGTACCGACACTT
GCCGATCTGCAATCGCTGTACGACGCCAGGCCCGGCGGCACCATGAATACGCAGCAGGG
CTGGCCGCTGGATGGTAAAAACTACCAGAACAGCACCGCTGATTTGAGCAGGAGTACGG
AAAACCGCTACGTTAAGTCGATTGATCTGCGCGATGGCGGTATTAGCTCGCTGGCGTGG
AGTGAACAACTCTATTTCGTCTGCTTGCAAAACGCCCATCCGGCGGCGACGCAGATAAC
GCTCACTTCGCCTTTGTATAACGACAGCGACGGATTTGCCAAAGCGAAAGTCGGCGAGA
CCATCCCGGTCATCATCACCACGCGGGACGCCCAGGGCAACCTGGCCGCCGACACGCCG
GTTATCTTTACGCGCGGCGACAGCGTCGGGCGCGCGAATCAGGAAGTGAATAGCGCGTC
GGCGGCGGAGATACAGATAAACCACAGCGACGGGCGTAGCAGCGGCGTTAAATATTACA
CCGCCACCGGCGCTGACGGCACGCTGACGCTGAATATCAGCCAGGACAGCGGCGCGGGC
TTTAAAACGCCGCTGACGGCGGTGATAGAGCACAACGGCGTAACGAGCGCGCTGCTACC
GGTCATTTTTACCGTCGTCACCAGCCCGGACACGCCGAAAGCCAACTACTGGGGCCATA
TGGCGGAAACGCTGACCGACAGCAGCGGCGTGGTGTACAAGCGCCCGTTGCTGGCCAGC
GAGTTCAGCGTCACGCCCGGCAAATCGCTGACCATCGCCAACGGTTACTACGACAAAGG
CGAAACCTGGGGCATGATAACCGTCGATAAAGCCTAGAACGGCGCCGGCGGCGGCTGCG
GGCGCAATTATCTGCCCACCGTCAGCAACCTCCAGACGCTGTACAGTACCTGGCCTGAT
AACGCCATGCGCAGTTGTAACGGCTGGCCGATGACCAGTTCTGGCAACAATAACGTCAG
CCGCTACTGGTGGGCGGGGGATTACGTGATGTCGTCAGACGGCGCGCAGTCGCTCTACG
CGGCGGTCAACCTGTTTAACGACGGCAATGATGTCAAAACGACGACCAGCACCACGACG
TACTATATGCAGACCTGCCTTACCTCGCCACGCAGCGCCGCCGCCAGCCTGACGCTGAC
GCTGGCAGGCCAGGATGCAGCCACCGGGATCGCAAAAGCGAAAAAGGGCGAGCAGATGG
CCGCCACCGTCACGGTGAAAGACGCCGCAGGTCAGCCGATGAAAAACGTGATGGTGAAA
ATCAGCCGGGGATCTTCGTACAACCGCGTCAACAGCGCCACATCGTCCTCAAGCGCTGC
AGACGATATTACGCTGCGTAATGTGATGCCGTCCGGCCCGGCGACATACCTGCTGGATA
CCTCCGCGAAATATCTGTATGCGCAAACCGATGCCCAGGGACAGGTGACATTTACCCTT
GCGCAGGACAGTACCGCAGGTTTAAAGACCACCATCAGCGCCGCCACGATGGACGGCAG
CAACCTGACAGACAGCAAAGACGCTATCTTTACCGTTATCACCAGCCCGGATAGCGACA
AGGCCTCATACTGGGGCCATATGCCGGAAACCTTTACCAACAGTAAGGGCGTTGAGTTT
GCGCGGCCGCTGCTGCGCGCCGAGCTTTCTTCAACGACGGATACCACGTCCTTCCTGTC
AAATAATGAGCACTGGTACACCTGGAACCGCTATCCAAACCTGTATCAGGACTCCGCCA
GCCCGTGCGATCGGCTGGGATTGCCGACGCTGGATGATTTAAAAACGTTGTATAACGAC
TATCCGAATGGTGGACTAACGGCCGCCTTTGGGTTGCCTGTAGACGCGGGGAAATACTG
GGGAGCAGGGGATTCGAAAGTCAACGATACGCACTCGACGAATAATTTCCAGTATATCA
GGCTAAATAACGGCGTTACGCAGGTCACAAATACGAATACCTCAACGGCGCAGCTCTGT
CTGGCGAAGCGCAGGGTGTTGGCGATAGCGTTGACCTCCAGCGCCATGAATGCCGAAAA
ATCCGCCGCGCTGGCGAAAAAAGGGGATAAAATCCCGCTGACGGTGACCGTCACGGACG
GCGCCGGCACGCCGTAG
SEQ ID NO: 2:
ATGAGTGTGTTGAGAAAACATTACTTGAAAGGGTATACCGCGCGGCAAATTGTACAGCG
AGCCATGAAAATTATTCCTTACTCGGTAAACGTCATGGATGAGCATGGCGTCATTATCG
CGTCTGGCGAACCTTCGCGGCTTCGCCAGCGTCACGAAGGGGCCATTCTGGCGCTGAAG
GAAAACCGTATTGTAGAAATTGATTCCGCTACCGCCAATCAGCTTAAAGGCGTGCGATC
CGGCATTAATCTTCCCATCTCTTTTCATGAACAGCTTATCGGCGTGGTCGGCATTACCG
GCGAACCGGAGGAGGTTCGCGCGTATGCCGAGCTGGTTAAAATGGCGGCGGAGCTGGTG
ATCGAGCATATGGTGCTGATCGAACAGTGACAATGGGATAAACGCTATCGCGAAGAGCT
GATCAACCAATTGATTTTGCGAGAAAATTCAACAGAGTCGTTGCGCTCCATGGCGGCCT
ATCTGGGCATTGATCTGGCGGTTCCCAGAGTGGTGCTGATTATTGAACTTTCCCAGCCG
GATCGCGAAGCGCTGCGCAATGTAATGGATTATTTCGAGAATCACGCGCGCAACCATTT
GGTGACGTTTACCGAATTTAATGAATTAATCATTATTAAGCCTATCACGTTAAAAGAGG
GAAAGTGGAATACCCGCCAGGAAATGGGCGAATTGCAGATTTTTAAATCATGGGCTGCA
TCATCGGGCTTTAGCCGTATTTTGGTTGGGGGCTATTTTGCCGGCGAGACGGGATTGCA
CCGATCTTTGCTCACAGCGAGAGCCACGCAGGCGATGGCGAAAAGACAAAAGCTGCGCA
GCCAGTACATTTTTTATCATGACCACGCGCTTCCCGCGCTGCTAAGCGGGCTGTCTGAA
AGTTGGCAAGTGCAGGAGTTATCGCGTCTGTGGCTGCAACTGGTGCAACATGACGCGAA
AGGCGTATTGCAACAGACGCTGCGGACTTGGTTTGAACATAATTGTGACCTGACGCAAA
CGGCCAAAGCATTGCATATTCATGTGAATACATTGCGCTATCGCTTACAGCGCTGTGAG
GATATTACGCACATAAAAATCAACGAGTTAAAAAGTACGCTTTGGCTTTATATCGGTAT
GGAGCTTCAGGCCGAATCTGTATCGTCCGACAAGTTACCGCTGCCTGGTCGAATCGAAA
TTTGTTGA
SEQ ID NO: 3:
ACCGATAAAGTCATCCCTGAACTAAAACAGTGGCAACAGAGCCCGCTGGAGAAGGTTTA
TCCCGTCGTCTGGCTGGACGCTATTCATTATAAAAACCGTGAGGATGGCCGTTATCAGA
GCAAGGCGGTTTATACCGTTCTGGCACTGAATCTAGAAGGCAAAAAAGAAGTTCTGGGC
CTATATCTGTCGGAAAGTGAAGGTGCTAACTTTTGGTTA
SEQ ID NO: 4:
AGTGCCAGAACGGTATAAACCGCCTTGCTCTGATAACGGCCATCCTCACGGTTTTTATA
A
SEQ ID NO: 5:
ATGCTGAAATTCTGAGCTTCATTCCTGAGCCTTGCTCTGATGTTGGCCGTTCCTTTTGC
CCCGCAGCCAGATCGTTCCTCAGGTGACCTCGAGTCCGGCAATTAAAAAAGCGGCTAAC
CACGCCGCTTTTTTTACGTCTGCACTCGGTACCAAATTCCAGAAAAGAGGCCTCCCGAA
AGGGGGGCCTTTTTTCGTTTTGGTCCCCGCGGTGCGGGTGCCAGGGCGTGCCCTTGGGC
TCCCCGGGCGCGTACTCCACGAAGTTCCTATACTTTCTAGAGAATAGGAACTTCGGAAT
AGGAACTTCATGCCTTTAATTAAGGGCGACCGTCTTCTATCGGTAATAACAGTCCAATC
TGGTGTAACTTCGGAATCGTCCCCAATTATTGAACACCCTTCGGGGTGTTTTTTTGTTT
CTGGTCTACCATCTCGTTGTGATAATAGACCTGAAGTGCCTACTCTGGAAAATCTTTGA
CAGCTAGCTCAGTCCTAGGTATAATGCTAGCAGCTGTCACCGGATGTGCTTTCCGGTCT
GATGAGTCCGTGAGGACGAAACAGCCTCTACAAATAATTTTGTTTAAAAAGAGGAGAAA
TAGTCCATGCGCTGATAGTGCTAGTGTAGATCGCTACTAGAGCCAGGCATTTTATATAC
TGGCTCGGGTAAGAACTCGCACTTCGTGGAAACACTATTATCTGGTGGAAGACGTGAGC
ACTAGTCTTGGACTCCTGTTGATAGATCCAGTAATGACCTCAGAACTCCATCTGGATTT
GTTCAGAACGCTCGGTTGCCGCCGGGCGTTTTTTATTGGTGAGAATCCAGGGGTCCCCA
ATAATTACGATTTAAATTGGCGAAAATGAGACGTTGATCGGCACGTAAGAGGTTCCAAC
TTTCACCATAATGAAATAAGATCACTACCGGGCGTATTTTTTGAGTTATCGAGATTTTC
AGGAGCTAAGGAAGCTAAAATGGAGAAAAAAATCACTGGATATACCACCGTTGATATAT
CCCAATGGCATCGTAAAGAACATTTTGAGGCATTTCAGTCAGTTGCTCAATGTACCTAT
AACCAGACCGTTCAGCTGGATATTACGGCCTTTTTAAAGACCGTAAAGAAAAATAAGCA
CAAGTTTTATCCGGCCTTTATTCACATTCTTGCCCGCCTGATGAATGCTCATCCGGAAT
TTCGTATGGCAATGAAAGACGGTGAGCTGGTGATATGGGATAGTGTTCACCCTTGTTAC
ACCGTTTTCCATGAGCAAACTGAAACGTTTTCATCGCTCTGGAGTGAATACCACGACGA
TTTCCGGCAGTTTCTACACATATATTCGCAAGATGTGGCGTGTTACGGTGAAAACCTGG
CCTATTTCCCTAAAGGGTTTATTGAGAATATGTTTTTCGTCTCAGCCAATCCCTGGGTG
AGTTTCACCAGTTTTGATTTAAACGTGGCCAATATGGACAACTTCTTCGCCCCCGTTTT
CACCATGGGCAAATATTATACGCAAGGCGACAAGGTGCTGATGCCGCTGGCGATTCAGG
TTCATCATGCCGTTTGTGATGGCTTCCATGTCGGCAGAATGCTTAATGAATTACAACAG
TACTGCGATGAGTGGCAGGGCGGGGCGTAATTTGACTTTTGTCGGCTCGACCCACGACT
ATTGACTGCTCTGAGAAAGTTGATTGTTACGATTAGTCCGGCCGGCCGAAGTTCCTATA
CTTTCTAGAGAATAGGAACTTCGGAATAGGAACTACCGCGGTGCGGGTGCCAGGGCGTG
CCCTCGGGCTCCCCGGGCGCGTACTCCACGTCAGAGGAGCCAAATTCAAAAAAGCCTGC
TTTCTAGCAGGCTTTTTGCTTTCTAATGGAAGCATAAAAAAATGGCGCCGATGGGCGCC
ATTTTTCACTGCGGCAAGAATTACTTCAGACAGAGTATCAAAAGGCGAAACCTCCGCAA
TGCGGAGGTTTCTTTTTAAAGACTGCAGCAGCGATTGAGACTCAGCGAACCAACACCAA
TAAAGTGATCTATTCGAACCACCCGGATCTGGTGCGTCCGATTGCCTCGATAACCAAAT
TAATGACAGCGATGGTCGTGCTCGACGCGCGATTGCCGCTGGATGAAATACTGAAGGTC
GATATCAGCCAGACGCCGGAAATGAAAGGGGTTTACTCCCGCGTGCGCCTGAACAGCGA
AATTAGCCGCAAAAATATGTTGCTGCTGGCGTTGATGTCATCCGAAAACCGTGCGGCGG
CAAGCCTGGCGCACTATTATCCCGGCGGTTATAACGCATTTATTAAAGCGATGAATGCG
AAGGCGAAAGCGCTGGGCATGACGCATACCCGCTTTGTTGAGCCGACGGGGCTGTCGAT
TCATAACGTCTCGACCGC
SEQ ID NO: 6:
ATGTCTCGTTCTATCAGAATCTGTAGCTATCTGCTGCTGCCGCTGATCTACCTACTGGT
CAATGTCAAGATTGCCCAACTGGGGGAAAGCTTTCCCATTACCATCGTCACTTTTTTAC
CGCTGTTGCTGCTGTTATTTGTGGAACGCATTAGCGTAAAAAAATTGATGATCGCCTTA
GGCATCGGCGCGGGGCTTACGGCATTTAACTTCCTGTTCGGCCAGTCGCTGAATGCCGG
TAAATATGTCACGTCCACGATGCTGTTTGTCTATATTGTGGTCATTATCGGGATGGTCT
GGAGTATCCGATTCAAAACCATTTCCGCGCATAACCACCGAAAGATTTTGCGTTTTTTT
ATCTGGTGGTGGGTATAGTGGTCGCGCTCGCCGCGGTTGAGATGGCGCAAATTATCCTT
ACCGGCGGCAGCAGTATCATGGAAGGAATTTCGAAATATCTCATTTACAGTAATAGCTA
CGTACTGAACTTCATAAAATTTGGCGGTAAGCGTACCACTGCGCTTTATTTTGAACCGG
CATTTTTCGCTTTGGCACTAATCTCAATTTGGCTGAGCATCAAACAGTTTGGTATCAAA
ATACCGAAAAGCGATGCTATGATTCTGGCAGGGATAATATTATCAGGATCATTTTCAGG
GGTAATGACCTTTATCCTGTTTTACCTTCTGGAGTGGGCGTTCCAATATTTGAATAAGG
ATGCGATAAAGAAAAAACTTCCACTGGCGCTGGTATCATTAACCCTGTTTTTGGTTGGG
GTAATTATTGCATTTCCTTATATCGCGACACGACTTGGCGATTTAGGGACGGAAGGATT
ATCTTCTTATTATCGTATTGTGGGCCCGTTAGTCATGGTCGGATATTCCTTGACCCATA
TTGATGGTGTAGTCAGATTTGGCTCACTTTATGAATATGTCGCATCATTCGGAATCTTT
AACGGTGCGGATGTCGGGAAAACCATAGACAATGGATTGTATCTGCTGATTATTTATTT
TTCCTGGTTCGCAGTGCTAATGACGCTGTGGTATATGGGGAAAGTTTTAAAAATGGCGC
TAAATGCGTTTGGCGATAATCGCAATTTTCGGGTGCAGCTCTATCTTTTTACGCCGGTG
TCGCTGTTTTTTACCGGTTCAATATTTAGCCCGGAATATGCTTTTTTAATCGTCTGTCC
GTTCATTTTGCGCAAGGCGTTAAAAATTTCATAA
SEQ ID NO: 7:
ATGAAATTATTAATTTTAGGCAACCACACATGCGGCAACCGTGGTGATAGCGCCATTAT
GCGCGGTTTGCTTGATGCCATCCGTCAACAGGCGCCAGAGGCGGAGATGGATGTGATGA
GCCGTTTCCCGGTGAGTTCCTCCTGGTTGCAGGGGCGACCGATTATTGCCGACCCGCTG
TATCAATTAAGCCAGAAACAGCAGGCGGCGGCAGGGCTTAACGGTCGGGTGAAAAAGTA
TTACGCCGTCGTTTTCAGCATAAAATTTTATTGTCTAAAGTCGCCCAGGAAGGATCGCT
GCGTAATTTTGCTATCGCGCCGGAATTTGCCGAATTCGCGCAATTTATTGCGCAGTATG
ACGCCGTCATTCAGGTGGGCGGTTCAAACTATGTCGACTTATACCCAGATCGTTCCTCA
GGTGACCTCGAGGTGAAGTGAAAAATGGCGCACATTGTGCGCCATTTTTTTTGTCTGCC
GTTTACCGCTTCTCTGAAAATCAACGGGCAGGTCACTGACTTGCCCGTTTTTTTATCCC
TTCTCCACACCGCGGTGCGGGTGCCAGGGCGTGCCCGTGGGCTCCCCGGGCGCGTACTC
CACGAAGTTCCTATACTTTCTAGAGAATAGGAACTTCGGAATAGGAACTTCTTGTGTCT
CAAAATCTCTGATGTTACATTGCACAAGATAAAAATATATCATCATGAACAATAAAACT
GTCTGCTTACATAAACAGTAATACAAGGGGTGTTATGAGCCATATTCAGCGTGAAACGA
GCTGTAGCCGTCCGCGTCTGAACAGCAACATGGATGCGGATCTGTATGGCTATAAATGG
GCGCGTGATAACGTGGGTCAGAGCGGCGCGACCATTTATCGTCTGTATGGCAAACCGGA
TGCGCCGGAACTGTTTCTGAAACATGGCAAAGGCAGCGTGGCGAACGATGTGACCGATG
AAATGGTGCGTCTGAACTGGCTGACCGAATTTATGCCGCTGCCGACCATTAAACATTTT
ATTCGCACCCCGGATGATGCGTGGCTGCTGACCACCGCGATTCCGGGCAAAACCGCGTT
TCAGGTGCTGGAAGAATATCCGGATAGCGGCGAAAACATTGTGGATGCGCTGGCCGTGT
TTCTGCGTCGTCTGCATAGCATTCCGGTGTGCAACTGCCCGTTTAACAGCGATCGTGTG
TTTCGTCTGGCCCAGGCGCAGAGCCGTATGAACAACGGCCTGGTGGATGCGAGCGATTT
TGATGATGAACGTAACGGCTGGCCGGTGGAACAGGTGTGGAAAGAAATGCATAAACTGC
TGCCGTTTAGCCCGGATAGCGTGGTGACCCACGGCGATTTTAGCCTGGATAACCTGATT
TTCGATGAAGGCAAACTGATTGGCTGCATTGATGTGGGCCGTGTGGGCATTGCGGATCG
TTATCAGGATCTGGCCATTCTGTGGAACTGCCTGGGCGAATTTAGCCCGAGCCTGCAAA
AACGTCTGTTTCAGAAATATGGCATTGATAATCCGGATATGAACAAACTGCAATTTCAT
CTGATGCTGGATGAATTTTTCTAATAATTAATTGAGAAGTTCCTATACTTTCTAGAGAA
TAGGAACTTCGGAATAGGAACTACCGGTGCGGGTGCCAGGGCGTGCCCCAGGGCTCCCC
GGGCGCGTACTCCACGGACCAAAACGAAAAAAGGCCCCCCTTTCGGGAGGCCTCTTTTC
TGGAATTTGGTACCGAGTGCAGACGTAAAAAAAGCGGCGTGGTTAGCCGCTTTTTTAAT
TGCCGGACTGCAGCAGCGATTGAGACTCAGCGAACGGAGCGGGCGAAAGGGTTCACGAT
GATCAAATCTGCTCTCGACAGAATTCGGGAGGCGCAATGA
SEQ ID NO: 8:
AAGGCGGGCAACGGCATAGACATGAGTAACCTGTGCATCTTCGTTCTCACCCGGACGGT
GTACCAGCTTCTCTTCCAGACCAAACTCGAAGCTGAAATCGTCACCTTCACGGACAACG
CGCGCGGAAAGGCTGGCAATCTGTCCGGAACGGCGGGCAAGGTCGATCATTCCCCGGTA
GCCAATAATTAACTGAACGTTTTTTTGCCTGACTTTTCGTTTTTGTTTCCGAACGGCAG
CAGATAGGCATGACCGAGCGCGCCGCCGGGTTCCAGCCCAAGCTGGGAACACTGAACGA
TGGCGCTGACAAAACTCATGGTGTCACAGTCACCCAGCGCCGGAACTTTTCGGATTTCC
GTTGTGGC
SEQ ID NO: 9:
TTACTGCGTCGCTGCAGACGGCGTTTTTGTCGGCGTTGCGCTTAACGATGCCGGACGAG
TAGACGGTACGCTATCGCACGGTTTTTCCTGCGGACAAATCAGATCGAGCATTTTCAGC
GTCACGCCGTTGCTCCAGCCAAAGCCGTCCTGAAGGGGATATTCGCCGCCGCCACCGCC
GGTTCCGGTACTGCTGACGTCATATTTTTCGACCAGTTTTTTCTCGCGATCGTAGGTGT
GCTGCACATTGGTTAAAAAGCGCCAGGTGACTTCCATTGCCACGTCATCCTGCCCATAA
TTTTGCAATCCTTCGGCAGCGACCCATTGTAACGGCGCCCAGCCATTTGGCGCATCCCA
CTGCTGTCCGCTTTTAACCGAGGTGGTAGCCAGCCCGCCAGGCTGTAGCAGATGCGCCT
GGGCCGCCGCTGCCACTTTCGCGGCGCGATCTTTCGCGGCGGCGTTTACATAGAGCGGG
AACAGCGCGGCAGCGGTGAGTTGGTCACGGATTTTATTGTTCTGCAGATCGTAGTCGGC
ATACCAACCCTCTTTGTTATTCCACAGATGCATTTCAATGGCTTTTTGCCGCGCGTTGG
CCAGCGCGTCATATTGCGAGGCTTTGGCCCGATCGCCCGCCGCAGCGCTGGCGCGGGCG
AGGGTTTTCTCCAGCTGATACAGCAGAGCGTTAAGATCGACAGGGACAATCGTGGTGGT
ACGAATGGTACTGAGCTGCTGCGGATTATCCATCCAGCGGGAGCTGAAATCCCAGCCGG
AGGCGGCAGCAGATCGGAGGTCTCGATAGATCTCCGTTGCCGGGCGGCTGGGGTTGCTT
TTGGCGGTAGCGATATCTTCAACCCAGGATTCAGGGCGGGGCGTATCCCGATCGTCCCA
GTAGCGGTTGAGAACGCTGCCGTCCTCCAGTTTGACGACGCGTTGGTTTTGTTGCCCTG
GCTGCAATGTCTCAACGCCCTCCATCCAGTAGGCGTACTCTTTTTGCAGTTGCGGCAGG
TATTCTTTCAGCGCATCGTCACCTTCATGTTGCACCAGTAATTCAACCATAAACGCAAA
GAAAGGCGGCTGTGAACGACTCAGGTAGTAGGTACGGTTGCCGTTAGGAATATACCCCC
AGGCGTCAATTTCGTAACCAAAGTTCGCTACCATATCCGCCACCTTATCCCAGTGCCCG
CTTTCCGCCAGCCCCAGCATCGTAAAGTAGCTGTCCCAGTAGTAAATCTCCCTGAATCG
CCCACCCGGTACGACATAGGATTCAGGCAACGGCAAGAGCGAGTCCCACTTTTCGACGT
TTTTAGTTGAACGGGTCAGCACCGGCCACAGGCCATCAATATGTTCACGCAATGATTGC
CCGGCAGGCGGGACATATTTTTCACCCGCTTTCGGCAGGGTGAAGTTAACATCAACAAA
ATGACGCAAATCGAAGCCGGACTAGTTCCGCTGCATACGATAATCCGCAAGAATCATGA
GCGGATCGCTATTAGGTATGGCGTCAGCAAAGGTTTTCTGATCGGGGAAGAGTTTTGCA
TTCTGGACATCATTAAAGAGCGGGCCAAGCAAAATATCCGGCGGCTGCGGAGTTTCAGT
GTCGGAAGACGGGTCTGCGGCAGTCGCCGAAAACGATGCAAACGTCAGCAGCGTCCCTG
CCAGCGCCAGTTTTATGGCTTTCTGTAGTAGAACAGAACGGCGAATCTCTGGGGGTATC
AT
SEQ ID NO: 10:
TCATGCCTGTTTATCCTCATTGTCCGGCTGTTGTTCGGCTTGCGCTAACTGTTGTTTGT
GGCGTTTACGTTGGTTGAGCGCGCTGGCGACAGCGTGACCGCCAACGCCCGCAGCGACC
ACGCCCAGCGCGGTCAGCCCCACCGTATCGGCGGTTGAATGGGTACCCATCTGGGGAAT
ATCCACCACGCGGCTATAAAACGAGCCGCGATCCCAGAAACCATTTTCTGAACATCCCA
GACATCCGTGACCGGACTGGATAGGAAAGGAGACGCCGTCATTCCAGCGTGTGGAGGAG
CAGGCGTTATAGGTGGTTGGCCCTTTACAGCCCATCTTGTACAGGCAGTATCCCTTGCG
GGCGGCGTCATCATCCCAGCTCTCGACAAATTCACCGGCATCAAAATGGGCGCGACGGT
AGCATTTATCGTGGATACGCTGACCATAGAACATCAGTGGACGGCCCATGCGATCGAGT
TCCGGCAGACGATCAAACGTCACCATATAGGTGATAATGGCGCTCATGACATCCGGGAT
TGGTGGACATCCAGGGACTTTCACGATCGGCTTGTCGGTGATCACTTTATCGATAGGCG
TTGCCTGGGTCGGATTGGGGCGGGCGGCCTAGACGCAACCCCAGGAGGCGCAGTTTCCC
CAGGCGATAATAGCGCTGGCGCCCGCGGCGGCTTTCTTCAGTTTTTCAATAAACGGGCG
GCCGCCGCTGATACAGAACATTCCTTGCTCTCCTAACGGCGGATTGCCTTCCACCGCCA
GAATGTATTTCCCGGCGTAGCGAGTGGTAATATCGTCAAAGACTTCTTCGGCCTGTGCG
CCGGCGGCGGCCATCAGGGTGTCGTCATAATCGAGGGAAATCAGCGAGAGGATCACATC
TTTGGCTAGCGGGTGCGAGGAACGGATAAAGGATTCGGTACAGCAGGTGCATTCCAGTC
CATGAATCCAGACCACCGGAATCCGCGGTTTATTCTCCAGCGCCCAGGCGATCTTTGGC
GTCATTCCGGCGCCCAGTCCCAGCGATGTGGCGGCAAGGCTACAGAATTTGAGAAAGCT
GCGTCGGGTCACTCCCTTACGACGCATGGCTTGATAAAAGGTCTCCTCGTTATTCAT
SEQ ID NO: 11:
ATGCGGGTGATTCGTCCTGTCGAACATGCGGATATCGCCGCGCTGATGCAGCTTGCAGG
CAAAACGGGCGGCGGCTTAACCTCGCTACTGGCAAATGAAGCGACGCTGGCGGCCCGCA
TTGAACGCGCGCTAAAAACCTGGTCCGGCGAGCTGCCAAAAGGCGAACAAGGATATGTA
TTTGTTCTCGAGGACAGTGAAACGGGTGAGGTCGGCGGGATCTGCGCCATTGAGGTCGC
GGTCGGCCTTAACGACCCTTGGTATAACTATCGTGTCGGTACGCTGGTTCATGCTTCCA
AAGAGCTGAATGTATATAACGCGTTGCCAACATTATTCCTGAGTAATGATCATACCGGC
AGCAGTGAGCTTTGCACGCTGTTTCTCGATCCCGAATGGCGTAAAGAGGGTAACGGCTA
CTTGTTGTCAAAATCGCGCTTTATGTTTATGGCCGCGTTTCGCGATAAATTCAATGAGA
AAGTGGTGGCGGAAATGCGCGGTGTCATTGACGAGCATGGTTATTCTCCCTTCTGGCAA
AGTCTCGGCAAACGCTTTTTTCTATGGATTTCAGCCGGGCGGATTTTTTATGCGGTACC
GGGCAAAAAGCGTTTATCGCCGAGCTGATGCCCAAACATCCTATTTATACCCACTTCTT
ATCTGAAGAGGCACAGGCCGTGATTGGCGAAGTCCACCCGCAAACGGCGCCAGCCCGCG
TGGTGCTGGAAAAGGAAGGCTTTCGCTATCGCCACTATATCGACATCTTCGACGGTGGG
CCGACGCTGGAGTGTGATATTGACCGCGTGCGGGCTATTCGTAAAAGCCGACTGGTGGA
AGTCGCGGAAGGGCAACCCGCGCTTGGCGACTATCCTGCATGTCTGGTCGCCAATGAAA
ACTATCACCACTTTCGGGCCGCGCTGGTGCGTGCCGATCCGCAGACTTCACGACTTGTA
CTTACCGCCGCGCAGTTGGATGCGCTGAAATGTCGCGCGGGCGACCACGTTCGGCTGGT
ACGCCTTTGCGCTGAGGAGAAAACCGTATGA
SEQ ID NO: 12:
ATGCTGTACAGGGCGCGGCCACGCCCCGTACAGCGGGTACCACAGATAAAACGAGCCTC
AGTGAGGAAGAGAATATGTTACTCAAAAAACAGGTGTTATTTCCAGCAACCAAAAAAGC
GTTTGGTCTTTTCCGTGACCCGTTCGCCGACGAAGCCATGCAGGGTTCTGATGATGTGT
TCACCACACCAGACATTCGCTACGTGCGTGAGGCGTTGTACCAGACAGCCCGTCATGGT
GGGTTTATGGCCGTCATCGGTGAGTCCGGTGCGGGTAAATCCACGCTGCGCCGTGACCT
GACTGAACGTATCAACCGCGAGAATGCGCCAGTGATTGTTATCGAGCCATACATCATCG
CTATGGAAGACAACGATGTGAAAGGGAAAACTCTGAAGGCAGCAGCGATTGCCGAAGCC
ATTATCAGTACCATCGCACCACTGGAAAGCATCAGACGCAGTCAGGACGCCCGCTTTCG
CCAGTTGCATCGCGTCCTGAAAGACAGCAACCAGGCGGGGTTCAGCCACGTTCTGGTGA
TTGAGGAGGCTCACAGTTTGCCCATTCCGACACTGAAACACCTCAAACGCTTTTTTGAG
CTGGAGTCCGGTTTCAAAAAGCTGCTGTCCATCGTGCTGATTGGCCAGCCGGAACTGGC
GACAAAACTCTCTGAACGCAACATGGAAGTCCGCGAAGTCGTTCAGCGCTGTGAGGTGG
TCGAACTTCTGCCTCTGGACAATCACCTTGAAGAATTTCTGACGTTCAAACTGCAACGG
GCCGGTAAACAACTGACGGACATTATGGACGCCAGCGCAGTGGATGCCATACGTACCCG
CCTGAGCAATCCGGGAAGTCATCGTAAAAATATGGTCAGCCTGCTGTATCCGCTGGCCG
TCAGTAACCTGGTAATAGCCGCCATGAATCTGGCCGCTGAAATCGGGGTTCCACAGGTC
AACGCTGACGTTGTCAAAGGGGTTTAA
SEQ ID NO: 13:
ATGAAAAAAGTAGTGGTGTTATCTGCGGTAGCCGCAGCCGTGATGATGGCTGGAGCCGC
CAACGCAGCAGAAATCTATAACAAGGATGGCAATAAACTGGATTTGTATGGCAAGGTCG
ACGGCCTGCATTATTTTTCCAGTAATCATAGTACGGATGGCGATCAATCTTATATCCGT
ATGGGTATTAAAGGCGAGACTCAAATTACCGATCAACTAACCGGTTTTGGACAGTGGGA
GTATCAGGTCAACGCCAATCGTCCGGAAGATGGTGACTCCAGCGGTTCCCCGCAAAGCT
GGACGCGTCTCGGTTTTGCTGGTTTGGCATTTGCCGATATGGGGTCTGTTGATTATGGT
CGTAACTATGGCGTGTTATACGACATTGGTTCATGGACTGACGTACTGCCTGAATTTGG
TAATATTACCTGA
SEQ ID NO: 14:
TTTTAATAGCCGTACCCGGCATGACTTGCCTTTGATCTTCCCGTTTTGCAACTGCTTCC
AGGCTTTTTGCGCTACTGCTTGACGTACGGCGACGTAAACGTGCATGGGATGCACGTTA
ATTTTGCCAATATCCGCCCCGTCCAATCCAATATCGCCGGTCAGCGCGCCTAAAATATC
TCCCGGACGCATTTTCGCTTTTTTGCCGCCGTCAATGCATAGGGTAGCCATCTCTGCGG
CCAGAGGGAGTAACGGCTGCCGGGCGGGCGCATTCAGCCAGTTCAGCTTGAGTTGCAGC
ATTTCTGAAAGAATATTCGCCCGCTGCGCCTCTTCCGGCGCACAGAAACTGATCGCCAG
GCCGCTGCTTCCCGCGCGCGCCGTACGGCCAATACGATGGACATGCACCTCCGGGTCCC
AGGCCAGCTCATAGTTAACCACCAGTTCGAGCGATTTAATGTCTAACCCTCGCGCGGCA
ACGTCGGTGGCAACCAGAATGCGCGCGCTGCCATTTGCAAAACGCACCAACGTCTGGTC
GCGGTCGCGTTGTTCCAGATCGCCGTGGAGCGCCAACGCGCTTTGTCCTACCGCATTAA
GCGCATCACAAACGGCCTGACAATCTTTTTTGGTATTGCAAAACACCACGCAGGACGCT
GGCTGATGCTGGCTAAGCAACGTTTGTAGCAGCGAAATTTTTTCATGCGCCGACGTTTC
GAAGAACTGCTGTTCGATAGCCGGTAGCGCATCTACCGTATCGATTTCAATACGTATTG
GCTGCTGCTGTACACGACCGCTAATCGCCGCAATGGCCTCAGGCCAGGTTGCTGAAAAC
AACAACGTCTGGCGCGTCGCAGGCGCAAAGCGGATCACCTCATCAATGGCGTCACTGAA
TCCCATGTCCAGCATTCGGTCTGCTTCATCCATTACCAGAATGTGCAGCGCATCCAGCG
ATACGGTTTCTTTTTGTAAATGATCCAGCAGGCGCCCCGGCGTCGCGACAATGATATGC
GGAGCGTGCTGAAGCGAGTCGCGCTGTGCGCCAAAGGGCTGCCCGCCACACAAGGTCAG
AATTTTGGTATTTGGCAGAAAACGGGCCAGGCGACGTAACTCCCCGGCTACCTGATCCG
CCAGCTCCCGCGTCGGGCACAGCACTAATGCCTGTGTCTGGAACAGAGTGACATCAATT
CGATGCAAGAGCCCAAGACCAAACGCCGCCGTTTTACCGCTACCGGTCCTGGCCTTGCA
CACGCACATCATTACCCGCCAGAATGACGGGTAATGCTGCGGCCTGGACAGGCGTCATC
TCAAGATAGCCCAGCTCAGTAAGGTTATTGAGCTGGGCGGCGGGCAAAACATTCAGGGT
TGAAAAAGCGGTCAC
SEQ ID NO: 15:
TTCTTTTACGGTTAACGCTTCTGTTTTTTTCTTCTTCTCCAGCACCATCTGGCGACGAG
GATCGAGTCCCTCAGTTAACCATGATCTAAAGTACTGACGGCGTTCCCTGGCATGAGAT
AGTGATATGGTTGGATAATCGCCAATCGTCAGTTGAGCAGCGTTCCCGTTCCATCTGTA
TCGGTAAAAGAATGTTATACTGCCGGATGTAGAGAGCCTGACATTGAGACCATGAGCGT
CTGAGATGACCTTGATTTGGTCTCTTTTTTTGCCAGGAGCCTTTCTTAATTTTGTATCG
GCAAGCAATGTGTACACTCCATAAGAGGATATACACATCTGTGTATACAT
SEQ ID NO: 16:
GTGTCTCAGCCTTGTCCCTGTGGTAGCGCTGACGAGTATAGCCTATGTTGTGGTCGTAT
TGTGTCCGGAGAACGAGTAGCACCCGATCCGTCACATCTCATGCGCTCTCGTTACTGCG
CTTTTGTGATGAAAGACGCAGATTACTTAATTAAGAGCTGGCATCCAACTTGCAATGCG
GCCGCGTTTCGTGATGATATCATCGCCGGATTTGCCAATACCAGGTGGCTCGGCCTGAC
TATTTTTGAACATACCTAGTCAGAAGCAGAAAATACAGGGTATGTTAGTTTTATCGCGC
GTTTTTCCGAACAAGGGAAAAACGGGGCGATTATCGAACGTTCTCGTTTTATCAAAGAA
AACGGTCAGTGGTATTATATTGACGGTACCCGCCCGCAGTTGGGTCGAAATGATCCCTG
CCCGTGCGGTTCAGGCAAAAAATTTAAAAAGTGCTGCGGCCAGTGA
SEQ ID NO: 17:
ATGTCTTTCATTCAATACCGCCGGGACAAACACCTTCCGTCCACCGCCGCACCGTCGCT
GTTAGCTATGGGTATGGCGATGGCATTTATGCCTGCCGCCTTCGCGGCTGAGGATACCG
TTATTGTCGAAGGCGCGACGACCGCTGACGCCATAAACCGTGAAGAACAGGATTACAGC
GTGAAAACGACCGCAGCGGGCACCAAAATGCCGATGACTCAGCGCGATATCCCGCAGTC
GGTCAGTATTGTCAGCCAACAGCGTATGGAAGACCAGCAGTTGCAAACCCTGGGCGAGG
TGATGACCAATACGCTGGGGATCAGCGGAAGCCAGGCTGACTCCGATCGCATCAGCTAT
TACTCGCGCGGGTTTGAAATTGACAACTATCCAGATCGTTCCTCAGGTGACCTCGAGAG
TTAACCAAAAAGGGGGGATTTTATCTCCCCTTTAATTTTTCCTCGCAGATAGCAAAAAA
GCGCCTTTAGGGCGCTTTTTTACATTGGTGGCGGTGCGGGTGCCAGGGCGTGCCCCTGG
GCTCCCCGGGCGCGTACTCCACGAAGTTCCTATACTTTCTAGAGAATAGGAACTTCGGA
ATAGGAACTTCTAGTAGCCCGCCTAATGAGCGGGCTTTTTTTTAATTCCCCTATTTGTT
TATTTTTCTAAATACATTCAAATATGTATCCGCTCATGAGACAATAACCCTGATAAATG
CTTCAATAATATTGAAAAAGGAAGAGTATGAGCATTCAGCATTTTCGTGTGGCGCTGAT
TCCGTTTTTTGCGGCGTTTTGCCTGCCGGTGTTTGCGCATCCGGAAACCCTGGTGAAAG
TGAAAGATGCGGAAGATCAACTGGGTGCGCGCGTGGGCTATATTGAACTGGATCTGAAC
AGCGGCAAAATTCTGGAATCTTTTCGTCCGGAAGAACGTTTTCCGATGATGAGCACCTT
TAAAGTGCTGCTGTGCGGTGCGGTTCTGAGCCGTGTGGATGCGGGCCAGGAACAACTGG
GCCGTCGTATTCATTATAGCCAGAACGATCTGGTGGAATATAGCCCGGTGACCGAAAAA
CATCTGACCGATGGCATGACCGTGCGTGAACTGTGCAGCGCGGCGATTACCATGAGCGA
TAACACCGCGGCGAACCTGCTGCTGACGACCATTGGCGGTCCGAAAGAACTGACCGCGT
TTCTGCATAACATGGGCGATCATGTGACCCGTCTGGATCGTTGGGAACCGGAACTGAAC
GAAGCGATTCCGAACGATGAACGTGATACCACCATGCCGGCAGCAATGGCGACCACCCT
GCGTAAACTGCTGACGGGTGAGCTGCTGACCCTGGCAAGCCGCCAGCAACTGATTGATT
GGATGGAAGCGGATAAAGTGGCGGGTCCGCTGCTGCGTAGCGCGCTGCCGGCTGGCTGG
TTTATTGCGGATAAAAGCGGTGCGGGCGAACGTGGCAGCCGTGGCATTATTGCGGCGCT
GGGCCCGGATGGTAAACCGAGCCGTATTGTGGTGATTTATACCACCGGCAGCCAGGCGA
CGATGGATGAACGTAACCGTCAGATTGCGGAAATTGGCGCGAGCCTGATTAAACATTGG
TAAAGAAGTTCCTATACTTTCTAGAGAATAGGAACTTCGGAATAGGAACTACCGGTGCG
GGTGCCAGGGCGTGCCCCCGGGCTCCCCGGGCGCGTACTCCACGGACCAAAACGAAAAA
ACACCCTTTCGGGTGTCTTTTCTGGAATTTGGTACCGAGCCTTTGGTCGAAAAAAAAAG
CCCGCACTGTCAGGTGCGGGCTTTTTTCTGTGTTTCCCTGCAGCAGCGATTGAGACTCA
GCGAACGGTTGACGGTATTCCAACGTATTTTGAGTCCCGCTGGAATCTGGGCGATGCGC
TAACGGATACCGCGCTGTATGAGCGCGTGGAGGTGGTTCGCGGCGCGAATGGGCTGATG
ACCGGAACCGGCAATCCTTCTGCCTCAATTAATATGATCCGTAAACACGCCACCAGTCG
GGAGTTCAAAGGTAACGTCTCCACAGAATACGGTAGCTGGAATAAGCAGCGTTACGTCA
TGGATCTGCAAAGTCCGCTCACCGCAGACGGTAATGTCCGTGGGCGCATCGTGGCGGGT
TATCAGAATAATGATTCCTGGCTGGACCGCTATAACAGTGAGAAAGCGTTCTTTTCCGG
AATTATCGATGCCGATCTGGGTGCAACTACCAACCTTTCAGCCGGTTATGAATATCAAA
AAATTGATGTCAACAGCCCGACCTGGGGCGGTCTGCCGCGCTGGAATACCGATGGCAGC
AAAAATAGCTATGACCGCGCGCGTAGTACCGCCCCGGACTGGGCTTATAACAATAAAGA
GATAAATAAATTCTTCGTCACGCTTAAGCAGCGTTTTGCCGAAAGCTGGCAGGCGACCC
TGAATGCGACCCATACTGAGGGTCAAATTCGACAGTAAAATGATGTATATCGATGCTTT
AGTGGATAAAGAGACAGGTACGCTGGTGAGTCCTTATGGCGCAAGCTACCCCGTGGTCG
GCGGTACTGGCTGGAACAGCGGCAAGCGCAAAGTAGATGCCATAGATCTCTTTGCTGAT
GGCGCTTATGAGCTGTTTGGCCGTCAGCACAATATGATGTTTGGCGGCAGTTATAGTAA
ACAGAATAACCGCTATTTTAGCGCCTGGGCGAACGTCTTTCCGGACGACATTGGCAACT
TCGGCGCCTTTAACGGTAATTTCCCCCAAACCCACTGGGCGCCACAAAATCTGGCGCAG
GACGATACCACGCATATGAAATCGCTGTATGCCGCTACGTGCATTTCGCTGGCCGACCC
GCTGCATCTGATTCTTGGCGCTCGTTATACCAACTGGCGTGTTGATACACTGACCTACA
GCATGGAGAAAAACCATACCACGCCGTATGCCGGGCTGATTTATGATATCAACGACAAC
TGGTCCGCTTATGCCAGTTATACCTCTATCTTTCAGCCGCAGAATAAACGCGACAAAGC
GGGTCAATATCTGGCTCCGATTACCGGTAATAACTATGAAGCGGGCCTGAAGTCCGACT
GGATGAACAGCCGCCTCACCACTACGTTGTCCGTGTTCCGCATTGAGCAGGATAATGTC
GCTCAAGCAACCACTATTCCCATCCCTGGCAGTAATGGGGAATTTGCCTGGAAGTCTAC
TGACGGAACGGTGAGCAAAGGCGTCGAATTTGAAGTGAACGGCGCAATTACCGATAACT
GGCAGATGACTTTCGGGGCTACCCGGTATGTTGCCGAGGACAATGAAGGTAACGCCGTG
AACCCTAATCTCCCGCGCACCAGCGTGAAGTTATTCACCCGCTATCGTCTGCCGGCGAT
ACCGGAGCTGACGGTCGGCGGCGGCGTGAACTGGCAGAATCGGGTTTACAAAGATACCA
CGACGCCATACGGCACCTTCCGCGCCGAACAGGGCAGCTATGCGCTGGTCGATCTGTTT
ACCCGCTATCAGGTGACGAAAAATTTCTCCGTACAGGGAAACATCAACAACCTGTTCGA
TAAAACCTACGACACCAATATTGATAGTTCCATTGTTTACGGCGCGCCGCGTAACGTCA
GCCTCACCGCCAACTATCAGTTCTGA
SEQ ID NO: 18:
ATGCAATTTAAAAATACTCCACAACGTTATGGCGTAGTTTCCGCCGCCCTCCACTGGCT
GACCGCCCTAGTGGTCTATGGCATGTTTGCGTTGGGTTTATGGATGGTCACGCTCAGTT
ATTACGACGGCTGGTATCACTAGGCGCCGGAAATACATAAAAGTATTGGCATGTTACTG
ATGATGGCGCTGATCGTGCGTATTATCTGGCGGCTTTATTCTCCGCCGCCCGTTGCGTT
GACCAGCTATTCCCGTTTAACGCGCATTGGCGCCGCCGCGGGTCATATCCTTCTGTATC
TCCTGCTCTTTGCGATAATCATTAGCGGCTACCTGATTTCCACCGCCGACGGTAAACCG
ATTAGCGTCTTTGGCTGGTTTGAGATTCCGGCCACGCTTACGGACGCGGGCGCGCAGGC
TGACATCGCCGGAACACTGCATCTGTGGTTTGCCTGGTCGCTGGTCATTATCTCGCTCT
CGCATGGGGTTATGGCGCTAAAACACCATTTCATCGATAAAGACGACACACTGAAACGT
ATGACAGGAATGTCGTCATCTGACTATGGAGCTCAAAAATGA
SEQ ID NO: 19:
TCAAACTCCCCATTTTGGGATAGGATGGTCGCCCATTGAGATGCAGACGTTTTTCATTT
CCGCAAACACTTCGAAGCTATATTCGCCGCCTTCGCGCCCGGTACCGGAGGCTTTCACG
CCGCCGAACGGCTGGCGCAGGTCGCGGACGTTCTGGGTGTTGACGAAGACCATGCCGGC
TTCAATCCCACGCGCCAGGCGCAACACTTTGCTCACGTCCTGGGTCCAGATATAAGAGG
CCAGACCGTATTCCACATCGTTCGCCAAACGTAACCCTTCCGCTTCGTCTTTGAATGGC
AGCAGGCAGGCGACCGGCCCAAAGATCTCTTCCTGCGCAACGCGCATACGGTTGTCGAC
ATCGGCCAGCACGGTTGGGCGCAGGAAGTTACCGCCTTTCAGATGCGCAGGCAGGTCAG
TGGGTTTTTCCGCACCGCCCGCCAGCAGCGTTGCCCCCTCTTCAATGCCGAGGCGGATA
TAACCGGAGACTTTCTCCCAGTGCTGTTGGCTAATCAGCGCGCCGACCTGGGTGTTCGG
GTCGGTCGGATCGCCGACACGCAGGCGATTCGCGCGTTCGGCAAAGCGCTTCACGAACT
CAGGGTAAATGCTCTGCTGGATAAAGATGCGCGACCCAGCGGTGCAGCGTTCGCCGTTG
ATCGAGAAGATGGTGAACAGCGCGGCGTCCAGCGCGCGCTCAATGTCGGCGTCTTCAAA
AATCAGCACCGGCGATTTGCCGCCCAGCTCCATCGAGTATTTTTTCAGCCCGGCATTTT
TCATGATATTGCGACCGGTGGCGGTACCGCCGGTAAACGACACCGCACGCACGTCATGG
TGGCGTACCAGCGCATCGCCCGCCGTCGCGCCGTAGCCCTGCACCACGTTCAGCACGCC
TGCCGGAATTCCTGCCTCCAGTGCCAGCTCGCCCAGCCTGTCGGCAGTCAGCGGCGACA
GCTCGGACATTTTGAGCACCGCGGTGTTACCCAGCGCCAGGCACGGCGCAACTTTCCAA
GTCGCGGTCATAAACGGCACGTTCCACGGCGACACCAGCGCGCAGACGCCGACGGGCTG
CACCAGCGTATAATTGAGCATTTTATCGTCAACCGGATAGGTCTTGCCGTTCATCTGCT
GGCACACTTCGGCGAAGAATTCGAAGTTATGCGAGGCGCGCGGGATCAGCACGTTTTAG
TCTGGTGAATAGGCAGGCCGGTGTCGGCGGTTTCCATCGCCGCGATTTCCGGCACATGC
TGGTCAATCAGGTCGCCAAGGCGGCGCATCAGGCGCGCGCGCTCTTTCATCGGCAGGTT
GGCCCATTTCGGGAACGCCTCTTTTGCCGCCGCGACAGCCTGGTTCACTTCTGCTTCAC
CGCCGGAGGCTACTTCCGCCAGCACATCACCGGTCGCCGGGTTAGTGGTCTGGAAGTAG
TCGTTACCTGCAACGTTTTTGCCGTTAATCCAATGATTTATTTTCTTCAT
SEQ ID NO: 20:
TTAACGTACCTGACTGGGCAGACACCCATAATGTTGGCGGAATCTGGCCGTAAATCGCG
ACCCCGACAGGTAGCCGCACCGTAGCGCTATTTCGCCGATCGGCAGCGTCGTCGATTGC
AGTTGAGAAAGCGCGCAGGACATGCGTACCTCTTCAATAATTTGCCGATAACTCTGCGA
TTCGCGCTGCAAACGCCGACGCAGCGTCGATGTTCCCATAGCAAGGCGGCGGGCTATCT
CCTGGGCTGTCCATAGCTTAGCAGGTGAGAGCATAATCAGTTGCCGCACCTGCTCCGTG
AGGGTGTAACGCCGTTCAATAAGCAGCGGTCCCGCCGCGCCATCATGTAAAAGCGCTAA
CAGTAACCCCATCGCCTGATGTTCCTGTAGCCCAACGGGCAACCCCTGGCGCACAGCAT
CCAACACGTTCTCCCACATAAATGTCAGGCTACGGCTCATCGGCGTACACAGCGATGTC
AGGTTTGCCGGTGGATAATCCTGAACATACATCGTTTTAAAGCGAGCAATAATCTCCGG
TGAAAGTAACAACAGGTCGGAGCGAAAACCGTTCTGCGCAGGCTGATTAATAAATATCC
TCCGGCATAGCCGGAGGTTTTTCTGATGCGCCTGTAAGGCTCTCTTACCAGCCGCGCCC
TAACAGGCGCATACGATCTGACATTTGCATCAAACTTCGTTACTTACGGCCCGTAAACA
GGCTGCCCGGATACGGGATCGATAATTGCTCACCCATTTTATCCTCTTCAAGCTGGTGC
TTTATGTAGTCCTGTATCTTCGCCGTATTCTTACCCACCGTATCGACATAGTACCCTCT
GCACCAGAACTCCCTGTTCCTGTATTTGAATTTTAGATCCCCAAACTGCTCGTAAAGCA
TCAGACTACTTTTACCCTTCAGATATCCCATGAAACTCGACACACTCATCTTCGGCGGG
ATCTCCAGAAGCATGTGAATATGATCTGCACAACATTCCGCTTCCAGAATTCGTACGTT
TTTCCATTCACACAATTTTCTTAATATGCTGCCTACTGCCCTACGCTTCTCTCCATAGA
ACGCTTGTCTTCGGTATTTGGGCGCGAAAACTATGTGATATTTACAGTTCCATCGGGTG
TGCGCTAAGCTCTTTTCGTCCCCCATTGGGACCCCCTTTTGATTTCTTGTTGAACTTTT
GCAGTTGCCAGACCGCAAGATGTTTTAACAAATCAAAAGGGGTTTTAATAACTGGCTTA
AAGCTGAAAGCTTTCCGGAACCCCCAGCCTAGCTGGGGGTTTTCCATAGACAATAATCT
CCAGCGGCGTGTTCGCCGGAATAATAATTAACTCGCCAGGCCCGGCGACAAGGCGGCTA
TCATCCTGAATGATGACTTTACTGCCCTGCGTAATATGGCAGATCGCGGCGGAAAAAAG
CGTAACACGATGCAAACGGTGCAGATGACTGGAACGCACCTCTGCCGTCGTAAGATCCT
GATGTCGAATATGCAT
SEQ ID NO: 21:
TCATAATAATTGCGGTGGCGCAAATCGTCGTTGCTGCAAAAGAGGAAGTTGCTCCCGCA
CCTGGTCTATTCGTTCTCTGAATATTTCCGCGACGATCAGCGCCGGACGATCGGCTGCC
GCGGCTATCGTTACCCCCAACGGTCGATAATACGACTTTGACCAATATTACGATTACCA
CACTCCCCGGCCGCAATCATATAGCAGGTGGTATCCAGCGCTCTCGCCGCCAGCAGCGT
CGACCACTGCTGCTCTTTCAACGGGCCGCGAACCCAGCCCGTCGGCAGCGCCAATACGT
CAGCCCCCTGTAAAGCCAGCGCTAACGCCATATCCGGAAAGCGCAGGTCATAACAGGTC
ATAAGACCCACCTTAACCCCCTCCACGTCCAGCACAGGCGCGATAACGGTTCCGGCATC
AATACTTTGGGATTCTTGCATCGAAAACGCATCATAGAGATGCAGCTTCGCGTAACGCG
CGACAATGTGACCCGCCCGTAGCGCCACCAGCATATTAACCGCCCGTCCAGGCGTTGAC
GGGACAAGGATAGTGAATATCGTCGTCATATTATTATGAGCGCTTTCTTCCAGAAGCCG
CGTCATAAACGCGCCATCCAGCGGCTGCGCGGCGCGAATCGGCAGGTCAAGGTCGATGT
CATCTCGCGCTAATATCCCCTCAGGCAACACCAGAAGCGATACGCCGCGGCCCGCCGCC
TGAGCCATTAACGAAACGCAAACTTGCACATTCTCTTCCCATACAGAACTCACCACAAA
CTGCCCGGCTGCAACGAACAT
SEQ ID NO: 22:
TCAGACTAACCGTTCATCTATCGCCAGCGCGTTTTCCATTTTACGCGAGACCATCGCGT
GACGCAGTAATACGCCGCCGCAGGCCATCATACCGCCAATCAACGCCGCCAGGATAATA
ATGATCGCTTTACCCGGGCCATCTTTTTTCACTGGCAGAGACGGCGACAGTTGATATTT
AAACGGGGTAAACTTCACGTCGCTCACATTCATTGCCGCCAGTTGTTCAACATAGTATT
GACGGTTACGCAAATCACCGTCGATCTCGGCCACGTCCGTTACCCCTTTTTCAATTTCC
AGTTTGCGGGAAATACCATCCGCGCCGAGGGAAATAGAAAAATCCGGATCATCTTTTAC
CGCCTGACCATTACTGTAAACCGGTCTCTTAATGCCAGCGGCGTTGGCGATTTCCAGCG
AATAATGAAGACGTTGAATATTGGCATCAAGCTGATTTTTGAGACGCACCCGATCCATC
GCCAGCTTTTCCTGCTCGTAGCGGGTTTTGATTTCCAGCTGGTTACGAATATTTTCCAG
CGTCTCTTTCACGACGATATCGGAGATGTACTGAATATAGCCAGCCAACACTTTTTGCG
CTTCTTCCCGCGTCGGCGCGGTAAAACTCAGCGTCCACGACGTGAATAACGACGTTTCA
TTTTTCTTGCCGGCATTGCTGTCCACCGCTTTCATTTTTTCGCTCAGCACGACAATCGC
CCGGTGAAGATCCTGCTCGTCTATTTGCGCGCCTTTTAATTGATCCATGACATACGGAG
AAGAACGAAGATATTCTTCCAGCAGCGAGGGCGAGCTAAACTTTTTAATAAACAGATTA
AATACGCTGGCCCGATCAACGCTTACCTCCATATCCAACACGCGCAGCGCGGTCAACGT
TCTCTCCAGCCCCTGCTACTGTACCGACTCCGCCGGTGTGACAATCGCCTGGCTGGTCC
ATTTTTGCGGCAGCAGAAAGGACAGAAGCAACCCCACGCACGCAAAGGCGAAAACGGTA
GCAAGAATACGACGTTTCGCCTGCCATAACACCTCTATAAGGCTAAACAAATCGATTTC
ATGACTGTTGGCGGGCGGCAGTGAGTAACCTGCAAATGACTGATTTTTCTCTTGTTTTA
CATTAAGAGATGGCAT
SEQ ID NO: 23:
TTAACCTGCGACTTTTTGCTCTCCTGTATTTTCAAAACCCGTGCGTTTCTTCAGTAGCG
CATAAGCAACGAACGCAACTATTACGCCTACAAACCAGGACACGCGGGATAAAGGCTCC
ATAAATGGTATAAACTTGCCGCCTAATGACAGAATGACTGCGACCAGGGTGACTGAAAA
TGCAGTCAGGTTAAATCCATTATCATAATATTTGTAATCACCGCTGGCGGTGTATAGCT
CATCAAGATTAATTTTTCCACGCATTACCACAAAATAATGAGCTAACATTACGCCGATT
ACCGGGCCAAGCATACCGCCGATAATATCGAGGAACAGATAAATACTGTCCTGATTCTC
CATTAATTTCCATGGACAAATCAGTAGACTGATAATACTGGCAATCATTACGCCATTTT
TATAGTTAAGCTTTGTCGGGGTAAGCGCCGCAATTTGATACCCCGCAGGTATAATATTA
CCGGTGGCGTTGGTTGAAATTGTCGTCATCAGAATCACCAGCACTGCAAAGAATGAAGC
AAACAGGCTGTCCCAGCGCTGCACAATATCCAGCACGTTCCAGGTATCCATACCATAAT
GAATACTGGCTCCGGCAATAATGCACACGCTGGCTACGGCGAATAATATATACGCTACG
ATAAGCCCCAATGTTTGTCCCAATGCCTGAGCGCGAAATGAATGCGCGTTTTGCGTGAA
ATCGGACGCGCTCACCGCTGGCGCAGCCCAGACGGCGACTACGGCGTTAATCACCACTA
GGAACAGAAAGCCGCTGTGCTCTGCTTTTTGCACGCCTGAAGGCAGATAGTCCAAAATC
GGGCCAATGCCGACCAGCGATATTGCCCAAATAGCCATGCCGCCAAAGACAATATAAAT
ACATGGATTGAGGATAGCGGTAAATTTATTTAATACTTTACCACCGCCAAAACCGATGC
CAACGTTAATGATCCAAAAAATTAGAAAAGTAATTAGCCCTGGCAGTGAAAAACCCAGC
AGCTTGAAATCTCCGCCTAATGTCTAAAATCCTGGCCAGATCTTCCCAATTAAAATAAG
AAATGCCAGCGATCCCGCGTAACACTGTAAGCCGAACCACATAATTGCCGCGATTCCCC
CTCGTAATAATCCAGGGAATAGCGCGCCGCGGACGCCGTAAGAACCTCGCAATATCATA
GCAAAAGGAACGCCATATTTGCTGCCTGCCGCGCCATTCATTACCATCGCCGCCGCAAT
AAATAATGCGCTGATAATAATGGCCAACATGATATTAAATGTTGATAGCCCCAGTATAA
AAAAACCGCCGACCATAACGTAATTTGGCACGTTATGTACAGATCCCATCCATAAGGTG
AAATAGTTAAATGCTTTCCAGTTTCGTTGGGTTTCTGTTTTAGGTAATAAGTCTTCGCT
ATAACCGCGTTGCTGATATAGCTCTCTTTGATGTTCCAT
SEQ ID NO: 24:
CTACAGCCAGTTCACCTCGCCATTTGGCGTAAACGCCGCCGCATCCAACGGTGAATTCT
GCTGAATAAACTCTTTCAGCACTTCCGCGTCAATAAACCCGGTATTCACGTAGCCCGGT
TTGTTATCGATGCGCGGATAACCATCGCCGCCCGTGGCGTTGAAACTCAGCGTCGCCAT
GCGGTAGGTTTTGGCCGGATCAACAGGCTCGCCTTTGATTTTCAGATCGGTGAGCTTGC
CCTCTTTGGCGACAAAGCTCACATTGGCGAACTGTGGATAGGCGCCGGAGTCCGGTTTC
ATCTGCGCTACGGCGGTGAGATAATCAACCACCTCTTTGCCGCTCATATCGGCATACAC
CACAATGTTGCCGAACGGCTGTACCTTGAGCACGCTTTTATAGGTAATATCTCCCGCCT
CAATCGAGTCGCGAATACCGCCGCCGCTCATCACGCCAAAATCGGCACCGGTGCGCGCG
ATCTGCGCAGCCAGAATCACCCGTCCCATATTGGTCTGGACAAATCTGACCTTACTGCG
ATCGCCTTCAAGCAGGCCATTCACGCTACCAATTTTCACCTCCAGTTGCGCTTTACCTT
TATTCTGGAACGGCGTTAATAACGAGAGCATTTGCGGATTTTCGGCGATTTCCGGCGTG
TAAAGTACACGCTCGCTTTTCCCGTTATCCCAGGTCACTTTTTTCTTGAGATTTACCGG
AATAAGCTGGTAGTTAACCATTTTCATCTCGCCGTTACGGAATTCGAAATCCGCGTGGC
CCACATATTTACCCCACTCATGCGCCTGCACGCTCCAGATGCCATTTTGCTTATCCGGC
GCGCAGGGCGTTCCCGTTACGTAATTCACCTGTTTTTATTTTCCGACGCCATGCATACC
GGGTCTTGTGAGTGACCGCCCACAATCATCGCCAGCGAACCGGCAGGCAGACTACGCGC
CATCTCAACGTCGCCCGGCGCATTCGAACCGTGATCGCCGTTGTCATAATGTCCCATAT
GCGTGGTCGCGATAATCACGTCCGGTTTTTCATTCATATTAAGTTCCTGAATCACCACC
TTTGCTTCTTCAGCAGGTTTACGAAACTCAATATCGGTGAAATATTCCGGGTTGCCTAT
TTTCGCCGTGTCATCGGTGGTTAAGCCGATTACCGCGATTTTTATATCCTGGCGTGTAA
AAATAGCCCACGGCTTAAACAGACGCTCGCCGGTACTTTTTTGATAAATATTGGCGGAA
AGAAAGGGAAACTTCGCCCACTTTTCCTGCTGGCGCAATACGGTGAGCGGATTATCAAA
TTCATGATTACCGACGGCCATAGCGTCGTAGCCAATCAGATTCATCCCGCGGAAATCGG
GCTCCACATCCTGGAGATCGGATTCCGGCACTCCGGTATTAATGTCGCCGCCGGATAAC
AACAGGACGCTTCCCCCCTCTTGCGCCACCTCTTTACGGATACTGTCCACCAGCGTTTT
TTGCGCCGCCAGACCATATTCGCCATATTCGCTGCGCCAGAAGTGACCGTGGTGATCGT
TGGTATGCAGGATAGTAATTTTATAGGTTTTATCTTTTTCGTAAGCCTGTGCAGGCTGA
GTCGTCAGCGCGAACGCCGCCAGTAACGCCAGCGCCACACCCCGTTTCAAAAATTTCAT
SEQ ID NO: 25:
ATGCTGTTACAGACGCTGGAAGAGAAGCTGGCGACGCTACGCCAACGCTGCGCGCCGCT
GGCGTAACATGCGACGTTAAGCGCGCGCTTTGACCGACACCTTTTCCGTACCCGCAGTA
CTCTGTTGCAGGGATATCTGGAAGAGGCAGACGCCAATCTCGCCGCGTTGCGTCAGGCG
GTAAAACATGAGCAACTGCCGCAAGTCGCCTGGCTGGCGGAACATCTGGCTTCGCAACT
GGAGGCGATATCGCGTGAAACCGCCGCCTGGTCGCTGCGCCAGTGGGATGCCGCCGCAC
CAGGGCTTGGCCGTTGGCAGCGCAGACGAATACAGCATCAGGAGTTTGAATGTCGACTG
CTGGCGATGACACAGGAGCGCAAAATTCGTCTGGCGCAGGCGACCGGCCTTGTCGAACA
ACAAACGCTGCAAAAGGAAGTCGAGATCTATGAAGGACGGCTGGCGCGCTGCCGACATG
CGCTGGAGAAAATAGAAAACGTACTGGCGCGTTTACCCCGTTAA
SEQ ID NO: 26:
ATGGCAATTAAATTAGAAGTGAAGAATCTGTATAAAATATTTGGAGAGCATCCGCAGCG
TGCCTTCAAATATATTGAAAAGGGACTATCGAAAGAGCAAATACTGGAAAAAACGGGGC
TATCGCTTGGCGTTAAAGACGCCAGTCTGGCCATTGAAGAAGGCGAGATATTTGTCATC
ATGGGATTATCCGGCTCGGGTAAATCCACAATGGTACGCCTTCTCAATCGCCTGATTGA
ACCCACCCGCGGGCAGGTACTGATTGACGGTGTTGATATTGCCAAAATATCAGACGCTG
AGCTTCGCGAGGTGCGCAGGAAAAAGATTGCGATGGTCTTCCAGTCATTTGCGCTCATG
CCGCATATGACCGTGCTGGATAATACGGCATTCGGTATGGAATTAGCGGGCATCGCGGC
GCAAGAGCGTCGCGAAAAGCGCTGGACGCCTTGCGTCAGGTGGGGCTTGAGAATTACGC
TCACGCCTATCCGGATGAACTTTCCGGTGGGATGCGTCAGCGTGTTGGGCTTGCCCGCG
CGCTGGCAATCAACCCTGATATCTTATTAATGGATGAAGCGTTTTCCGCCCTCGATCCA
TTAATTCGTACCGAAATGCAGGATGAGCTGGTGAAATTACAGGCGAAACATCAGCGCAC
CATTGTCTTTATTTCCCACGATCTTGATGAGGCTATGCGTATTGGCGACAGGATTGCCA
TTATGCAAAATGGCGAGGTCGTACAGGTTGGTACGCCGGATGAGATCCTGAATAATCCG
GCAAATGATTATGTCCGCACGTTCTTCCGTGGCGTGGATATTAGTCAGGTCTTTAGCGC
CAAAGATATTGCCCGTCGCAGTCCGGTCGGCTTAATTCGTAAAACGCCAGGTTTTGGTC
CCCGTTCGGCACTGAAATTATTACAGGATGAAGACCGTGAATATGGTTACGTCATTGAG
CGTGGCAATAAATTCGTGGGCGTCGTGTCCATCGACTCATTAAAAGCGGCATTAAGCCA
GGCGCAAGGGATTGAAGCGGCGCTTATCGACGACCCTTTAGTCGTTGATGCGCAAACCC
CACTCAGCGAGTTGCTCTCTCACGTCGGCCAGGCGCCCTGCGCGGTGCCGGTTGTCGAT
GAAGAACACCAGTATGTTGGCATTATTTCAAAACGTATGTTGCTACAGGCTTTAGATCG
CGAGGGGGGTAACAATGGCTGA
SEQ ID NO: 27:
ATGAAGTCATCTCATTTTTGTAAACTGGCAGTAACTGCATCTTTAGTTATGGGAATTGT
CTCCGGCGCTCAAGCCGCGGGTAGCAACACAGCAAAGGTTACTTTCCTTGGTAATATTG
TTGATTCCCCCTGCTCTGTCACATTGGATACGGAAGATCAAACAGTCAATATGGGCTCA
AGTATCGGTAATGGCACGCTGAGTAATGGTAAAACGACCATCAACAATGCCCGTACCTT
TCATATCGATCTTGAGGGTTGTACCTAGGCTACCGAGAAAAATATGAATGTGGTATTCA
CTACAGGTAGTGGAACCACAGCGGCTACAGGCGCCACGGATAATCTCGCGCTGATGAAG
ACTGACGGCACTGGCGCTATTAGCAACATAAGCCTGGCAATCGGCGATGCAGGCAAAAA
CAATATCAAACTGGGCGATACCTATACACAGGCCATTGCGGACCTGGACAGAGATACCA
TCCTTGATGAGAAGCAAAGCCTGAATTTCACCGCCTGGCTGGTTGGCGCAGCAACCGGC
ACCGTAGGCACAGGTGAATTCAGCAGCGCCGCCAACGTCACTATCTCTTACCTGTAA
SEQ ID NO: 28:
ATGGGTGAATTTTCGACACTTCTTCAGCAAGGAAACGGCTGGTTCTTCATTCCCAGCGC
CATTTTATTAGGTATTTTGCACGGGCTTGAACCAGGGCACTCCAAAACCATGATGGCGG
CTTTTATCATTGCCATTAAAGGTACGGTTAAACAGGCTGTCATGCTCGGTCTGGCAGCA
ACGCTTTCTCATACCGCGATCGTCTGGTTAATCGCGCTGGGTGGGATGTATCTTAGCCG
GGCATTCACCGCACAATCAGTGGAACCATGGCTGCAGTTAATTTCTGCGATCATTATTC
TGAGCACCGCGTGCTGGATGTTCTGGCGGACATGACGAGGCGAGCAGCAGTGGCTGGCG
GGAAACCACCATCACGACCACCATCACGACCACCATTACGACCATGACCATGACCATGA
CCATGACCATGACCATGACCATGACCATGACCATGACCATCATGGTCACATACATCCGG
AAGGCGCAACGTCAAAAGCGTATCAGGATGCCCATGAACGCGCCCATGCTGCCGATATT
CAACGCCGTTTTGATGGTCAAACAGTGAATAATGGACAGATCCTGCTGTTCGGCCTGAC
CGGAGGGCTTATCCCCTGTCCGGCTGCGATCACCGTTTTACTGATTTGTATCCAGCTTA
AAGCGTTTACGCTGGGTGCCACGATGGTGCTGAGCTTTAGTCTTGGCCTGGCATTAACG
CTGGTGACGGTAGGCGTTGGCGCGGCGATAAGCGTTCAACAGGCAGCAAAGCGCTGGAG
TGGTTTTTCGACGCTTGCCCGGCGGGCGCCCTATTTTTCGAGCATTCTGATTGGTCTGG
TCGGCGTGTATATGGGAATTCATGGCTATACCGGGATCATGCAGTAA
SEQ ID NO: 29:
ATGCCGCACTGCCAGGACAACACCAAACGTGAGTTCACACATCTGGTTAGAGTTTCTCT
GGCTTACCGCAAAATTGAGTGGGAACACGTTTCAACAGGCACTTCAGGTGCTGATGACT
GACGTGCGCCGCTGGAAGCATAA
SEQ ID NO: 30:
TCAATACACCTCCGAGGCTAAAAAGTAGCCTTCGCCATGCTGTGTGACCAGCAGTTCCG
GCGTAATTTTATGACGTAAGCGGCGAACCAGGACATCAATCGTGCGGAGATCCGGGGTC
TCTACGCGGCGGGCGGAAAGCATTCGCAGCAGACGTTCGCGATGCAGCACTTTGCCTGG
GTTGGTCACAAAGGCCAGCAGCAGCTCGTACTCCGCGCGCGTAAGCTTAATGGCTTCAC
CGTTGTGCTCCAGCGTGTGATTCATCACGTTCAGGCAGTAGCCGGAAAACATATAGCAG
TTTTCACTGGCATTTTGCGGCGTGGGGCGGGCCAGATCGATACGCCACAAAAGATTTTT
CACCCGCACTACCAGCTCGCGCAGTTCCAGCGGCTTGGTGACGTAGTCGTCCGCGCCCA
TCTCCAGCCCGACGATGCGGTCGATTTGGTCGCAACGCCCCGTCACCAGAATAATGCCC
ACCGTGGAGCGTTCGCGTAGCGCCCTGGTCAGCATCAACCCGTTTTCATCGGGGAGGTT
GATATCCAGCAGGATCAGCGAAACGTGCTCATGCTCCATGATGTCACGCGCAGGCCCGC
GCCGCTGTCGGTCACCGAAACGCGATACCCCTCCTGCTCAAAATAGGCCTGTAACCTGG
CCTGAGTAACAGGTTCATCCTCAACAATAACAATGTGATGTGACAT
SEQ ID NO: 31:
CCGCGGTGCGGGTGCCAGGGCGTGCCCTTGGGCTCCCCGGGCGCGTACTCCAC
SEQ ID NO: 32:
CCGCGGTGCGGGTGCCAGGGCGTGCCCGCGGGCTCCCCGGGCGCGTACTCCAC
SEQ ID NO: 33:
CCGCGGTGCGGGTGCCAGGGCGTGCCCCTGGGCTCCCCGGGCGCGTACTCCAC
SEQ ID NO: 34:
CCGCGGTGCGGGTGCCAGGGCGTGCCCTAGGGCTCCCCGGGCGCGTACTCCAC
SEQ ID NO: 35:
CCGCGGTGCGGGTGCCAGGGCGTGCCCATGGGCTCCCCGGGCGCGTACTCCAC
SEQ ID NO: 36:
CCGCGGTGCGGGTGCCAGGGCGTGCCCCCGGGCTCCCCGGGCGCGTACTCCAC
SEQ ID NO: 37:
GGCCGGCTTGTCGACGACGGCGTTCTCCGTCGTCAGGATCAT
SEQ ID NO: 38:
GGCCGGCTTGTCGACGACGGCGGCCTCCGTCGTCAGGATCAT
SEQ ID NO: 39:
GGCCGGCTTGTCGACGACGGCGCTCTCCGTCGTCAGGATCAT
SEQ ID NO: 40:
GGCCGGCTTGTCGACGACGGCGTACTCCGTCGTCAGGATCAT
SEQ ID NO: 41:
GGCCGGCTTGTCGACGACGGCGATCTCCGTCGTCAGGATCAT
SEQ ID NO: 42:
GGCCGGCTTGTCGACGACGGCGCCCTCCGTCGTCAGGATCAT
SEQ ID NO: 43:
CCGGCTTATCGGTCAGTTTCACCTGATTTACGTAAAAACCCGCTTCGGCGGGTTTTTGC
TTTTGGAGGGGCAGAAAGATGAATGACTGTCCACGACGCTATACCCAAAAGAAAAAAAA
AAAACCCCGCCCCTGACAGGGCGGGGTTTTTTTT
SEQ ID NO: 44:
TCCGGCAATTAAAAAAGCGGCTAACCACGCCGCTTTTTTTACGTCTGCACTCGGTACCA
AATTCCAGAAAAGAGGCCTCCCGAAAGGGGGGCCTTTTTTCGTTTTGGTCC
SEQ ID NO: 45:
TTCAGCCAAAAAACTTAAGACCGCCGGTCTTGTCCACTACCTTGCAGTAATGCGGTGGA
CAGGATCGGCGGTTTTCTTTTCTCTTCTCAACTCGGTACCAAAGACGAACAATAAGACG
CTGAAAAGCGTCTTTTTTCGTTTTGGTCC
SEQ ID NO: 46:
GCTGATGCCAGAAAGGGTCCTGAATTTCAGGGCCCTTTTTTTACATGGATTGCTCGGTA
CCAAATTCCAGAAAAGAGACGCTTTCGAGCGTCTTTTTTCGTTTTGGTCC
SEQ ID NO: 47:
GATCTAACTAAAAAGGCCGCTCTGCGGCCTTTTTTCTTTTCACTGTAACAACGGAAACC
GGCCATTGCGCCGGTTTTTTTTGGCCT
SEQ ID NO: 48:
AGTTAACCAAAAAGGGGGGATTTTATCTCCCCTTTAATTTTTCCTCGCAGATAGCAAAA
AAGCGCCTTTAGGGCGCTTTTTTACATTGGTGG
SEQ ID NO: 49:
GGAAACACAGAAAAAAGCCCGCACCTGACAGTGCGGGCTTTTTTTTTCGACCAAAGGCT
CGGTACCAAATTCCAGAAAAGACACCCGAAAGGGTGTTTTTTCGTTTTGGTCC
SEQ ID NO: 50:
TACCACCGTCAAAAAAAACGGCGCTTTTTAGCGCCGTTTTTATTTTTCAACCTTCCAGG
CATCAAATAAAACGAAAGGCTCAGTCGAAGACTGGGCCTTTCGTTTTATCTGTTGTTTG
TCGGTGAACGCTCTC
SEQ ID NO: 51:
ACATTTAATAAAAAAAGGGCGGTCGCAAGATCGCCCTTTTTTACGTATGACACAGTGAA
AAATGGCGCCCATCGGCGCCATTTTTTTATG
SEQ ID NO: 52:
TGCTCGTACCAGGCCCCTGCAATTTCAACAGGGGCCTTTTTTTATCCAATTCCATCGGG
TCCGAATTTTCGGACCTTTTCTCCGC
SEQ ID NO: 53:
CTTATTCCATAACAAAGCCGGGTAATTCCCGGCTTTGTTGTATCTGAACAATAAATGGA
TGCCCTGCGTAAGCGGGGCATTTTTCTTCCT
SEQ ID NO: 54:
AGCGTCAAAAGGCCGGATTTTCCGGCCTTTTTTATTAGGCAGCATGCTGCCAGGTGATC
CCCCTGGCCACCTCTTTT
SEQ ID NO: 55:
TAATCATTCTTAGCGTGACCGGGAAGTCGGTCACGCTACCTCTTCTGAAGAAACAGCAA
ACAATCCAAAACGCCGCGTTCAGCGGCGTTTTTTCTGCTTTTCT
SEQ ID NO: 56:
GTGAAGTGAAAAATGGCGCACATTGTGCGCCATTTTTTTTGTCTGCCGTTTACCGCTTC
TCTGAAAATCAACGGGCAGGTCACTGACTTGCCCGTTTTTTTATCCCTTCTCCACACCG
SEQ ID NO: 57:
TCTTTAAAAAGAAACCTCCGCATTGCGGAGGTTTCGCCTTTTGATACTCTGTCTGAAGT
AATTCTTGCCGCAGTGAAAAATGGCGCCCATCGGCGCCATTTTTTTATGCTTCCATTAG
AAAGCAAAAAGCCTGCTAGAAAGCAGGCTTTTTTGAATTTGGCTCCTCTGAC
SEQ ID NO: 58:
AAAGTTCTGAAAAAGGGTCACTTCGGTGGCCCTTTTTTATCGCCACGGTTTGAGCAGTG
CACTTGCTTAAAATCCCGCCAGCGGCGGGATTTTTTATTGTCCGGTTTAAGACA
SEQ ID NO: 59:
GCAGACAAAAAAAATGGCGCACAATGTGCGCCATTTTTCACTTCACAGGTACTATTGTT
TTGAATTGAAAAGGGCGCTTCGGCGCCCTTTTTGCATTTGTTGACGGCATATATTTGTA
TATCGAAGCGCCCTGATGGGCGCTTTTTTTATTTAATCGATAACCAGA
SEQ ID NO: 60:
CCAGATCGTTCCTCAGGTGACCTCGAGTCCGGCAATTAAAAAAGCGGCTAACCACGCCG
CTTTTTTTACGTCTGCACTCGGTACCAAATTCCAGAAAAGAGGCCTCCCGAAAGGGGGG
CCTTTTTTCGTTTTGGTCCCCGCGGTGCGGGTGCCAGGGCGTGCCCTTGGGCTCCCCGG
GCGCGTACTCCACGAAGTTCCTATACTTTCTAGAGAATAGGAACTTCGGAATAGGAACT
ACCGCGGTGCGGGTGCCAGGGCGTGCCCTCGGGCTCCCCGGGCGCGTACTCCACGTCAG
AGGAGCCAAATTCAAAAAAGCCTGCTTTCTAGCAGGCTTTTTGCTTTCTAATGGAAGCA
TAAAAAAATGGCGCCGATGGGCGCCATTTTTCACTGCGGCAAGAATTACTTCAGACAGA
GTATCAAAAGGCGAAACCTCCGCAATGCGGAGGTTTCTTTTTAAAGACTGCAGCAGCGA
TTGAGACTCAGCGAAC
SEQ ID NO: 61:
CCAGATCGTTCCTCAGGTGACCTCGAGGTGAAGTGAAAAATGGCGCACATTGTGCGCCA
TTTTTTTTGTCTGCCGTTTACCGCTTCTCTGAAAATCAACGGGCAGGTCACTGACTTGC
CCGTTTTTTTATCCCTTCTCCACACCGCGGTGCGGGTGCCAGGGCGTGCCCGTGGGCTC
CCCGGGCGCGTACTCCACGAAGTTCCTATACTTTCTAGAGAATAGGAACTTCGGAATAG
GAACTACCGGTGCGGGTGCCAGGGCGTGCCCCAGGGCTCCCCGGGCGCGTACTCCACGG
ACCAAAACGAAAAAAGGCCCCCCTTTCGGGAGGCCTCTTTTCTGGAATTTGGTACCGAG
TGCAGACGTAAAAAAAGCGGCGTGGTTAGCCGCTTTTTTAATTGCCGGACTGCAGCAGC
GATTGAGACTCAGCGAAC
SEQ ID NO: 62:
CCAGATCGTTCCTCAGGTGACCTCGAGAGTTAACCAAAAAGGGGGGATTTTATCTCCCC
TTTAATTTTTCCTCGCAGATAGCAAAAAAGCGCCTTTAGGGCGCTTTTTTACATTGGTG
GCGGTGCGGGTGCCAGGGCGTGCCCCTGGGCTCCCCGGGCGCGTACTCCACGAAGTTCC
TATACTTTCTAGAGAATAGGAACTTCGGAATAGGAACTTCCGGTGCGGGTGCCAGGGCG
TGCCCCCGGGCTCCCCGGGCGCGTACTCCACGGACCAAAACGAAAAAACACCCTTTCGG
GTGTCTTTTCTGGAATTTGGTACCGAGCCTTTGGTCGAAAAAAAAAGCCCGCACTGTCA
GGTGCGGGCTTTTTTCTGTGTTTCCCTGCAGCAGCGATTGAGACTCAGCGAAC
SEQ ID NO: 63:
AAATTCTGAGCTTCATTCCTGAGCCTTGCTCTGATGTTGGCCGTTCCTTTTGCCCCGCA
GCCAGATCGTTCCTCAGGTGACCTCGAGTCCGGCAATTAAAAAAGCGGCTAACCACGCC
GCTTTTTTTACGTCTGCACTCGGTACCAAATTCCAGAAAAGAGGCCTCCCGAAAGGGGG
GCCTTTTTTCGTTTTGGTCCCCGCGGTGCGGGTGCCAGGGCGTGCCCTTGGGCTCCCCG
GGCGCGTACTCCACGAAGTTCCTATACTTTCTAGAGAATAGGAACTTCGGAATAGGAAC
TTCATGCCTTTAATTAAGGGCGACCGTCTTCTATCGGTAATAACAGTCCAATCTGGTGT
AACTTCGGAATCGTCCCCAATTATTGAACACCCTTCGGGGTGTTTTTTTGTTTCTGGTC
TACCATCTCGTTGTGATAATAGACCTGAAGTGCCTACTCTGGAAAATCTTTGACAGCTA
GCTCAGTCCTAGGTATAATGCTAGCAGCTGTCACCGGATGTGCTTTCCGGTCTGATGAG
TCCGTGAGGACGAAACAGCCTCTACAAATAATTTTGTTTAAAAAGAGGAGAAATAGTCC
ATGCGCTGATAGTGCTAGTGTAGATCGCTACTAGAGCCAGGCATTTTATATACTGGCTC
GGGTAAGAACTCGCACTTCGTGGAAACACTATTATCTGGTGGAAGACGTGAGCACTAGT
CTTGGACTCCTGTTGATAGATCCAGTAATGACCTCAGAACTCCATCTGGATTTGTTCAG
AACGCTCGGTTGCCGCCGGGCGTTTTTTATTGGTGAGAATCCAGGGGTCCCCAATAATT
ACGATTTAAATTGGCGAAAATGAGACGTTGATCGGCACGTAAGAGGTTCCAACTTTCAC
CATAATGAAATAAGATCACTACCGGGCGTATTTTTTGAGTTATCGAGATTTTCAGGAGC
TAAGGAAGCTAAAATGGAGAAAAAAATCACTGGATATACCACCGTTGATATATCCCAAT
GGCATCGTAAAGAACATTTTGAGGCATTTCAGTCAGTTGCTCAATGTACCTATAACCAG
ACCGTTCAGCTGGATATTACGGCCTTTTTAAAGACCGTAAAGAAAAATAAGCACAAGTT
TTATCCGGCCTTTATTCACATTCTTGCCCGCCTGATGAATGCTCATCCGGAATTTCGTA
TGGCAATGAAAGACGGTGAGCTGGTGATATGGGATAGTGTTCACCCTTGTTACACCGTT
TTCCATGAGCAAACTGAAACGTTTTCATCGCTCTGGAGTGAATACCACGACGATTTCCG
GCAGTTTCTACACATATATTCGCAAGATGTGGCGTGTTACGGTGAAAACCTGGCCTATT
TCCCTAAAGGGTTTATTGAGAATATGTTTTTCGTCTCAGCCAATCCCTGGGTGAGTTTC
ACCAGTTTTGATTTAAACGTGGCCAATATGGACAACTTCTTCGCCCCCGTTTTCACCAT
GGGCAAATATTATACGCAAGGCGACAAGGTGCTGATGCCGCTGGCGATTCAGGTTCATC
ATGCCGTTTGTGATGGCTTCCATGTCGGCAGAATGCTTAATGAATTACAACAGTACTGC
GATGAGTGGCAGGGCGGGGCGTAATTTGACTTTTGTCGGCTCGACCCACGACTATTGAC
TGCTCTGAGAAAGTTGATTGTTACGATTAGTCCGGCCGGCCGAAGTTCCTATACTTTCT
AGAGAATAGGAACTTCGGAATAGGAACTACCGCGGTGCGGGTGCCAGGGCGTGCCCTCG
GGCTCCCCGGGCGCGTACTCCACGTCAGAGGAGCCAAATTCAAAAAAGCCTGCTTTCTA
GCAGGCTTTTTGCTTTCTAATGGAAGCATAAAAAAATGGCGCCGATGGGCGCCATTTTT
CACTGCGGCAAGAATTACTTCAGACAGAGTATCAAAAGGCGAAACCTCCGCAATGCGGA
GGTTTCTTTTTAAAGACTGCAGCAGCGATTGAGACTCAGCGAACCAACACCAATAAAGT
GATCTATTCGAACCACCCGGATCTGGTGCGTCCGATTGCCTCGAT
SEQ ID NO: 64:
TTTATTGCGCAGTATGACGCCGTCATTCAGGTGGGCGGTTCAAACTATGTCGACTTATA
CCCAGATCGTTCCTCAGGTGACCTCGAGGTGAAGTGAAAAATGGCGCACATTGTGCGCC
ATTTTTTTTGTCTGCCGTTTACCGCTTCTCTGAAAATCAACGGGCAGGTCACTGACTTG
CCCGTTTTTTTATCCCTTCTCCACACCGCGGTGCGGGTGCCAGGGCGTGCCCGTGGGCT
CCCCGGGCGCGTACTCCACGAAGTTCCTATACTTTCTAGAGAATAGGAACTTCGGAATA
GGAACTTCTTGTGTCTCAAAATCTCTGATGTTACATTGCACAAGATAAAAATATATCAT
CATGAACAATAAAACTGTCTGCTTACATAAACAGTAATACAAGGGGTGTTATGAGCCAT
ATTCAGCGTGAAACGAGCTGTAGCCGTCCGCGTCTGAACAGCAACATGGATGCGGATCT
GTATGGCTATAAATGGGCGCGTGATAACGTGGGTCAGAGCGGCGCGACCATTTATCGTC
TGTATGGCAAACCGGATGCGCCGGAACTGTTTCTGAAACATGGCAAAGGCAGCGTGGCG
AACGATGTGACCGATGAAATGGTGCGTCTGAACTGGCTGACCGAATTTATGCCGCTGCC
GACCATTAAACATTTTATTCGCACCCCGGATGATGCGTGGCTGCTGACCACCGCGATTC
CGGGCAAAACCGCGTTTCAGGTGCTGGAAGAATATCCGGATAGCGGCGAAAACATTGTG
GATGCGCTGGCCGTGTTTCTGCGTCGTCTGCATAGCATTCCGGTGTGCAACTGCCCGTT
TAACAGCGATCGTGTGTTTCGTCTGGCCCAGGCGCAGAGCCGTATGAACAACGGCCTGG
TGGATGCGAGCGATTTTGATGATGAACGTAACGGCTGGCCGGTGGAACAGGTGTGGAAA
GAAATGCATAAACTGCTGCCGTTTAGCCCGGATAGCGTGGTGACCCACGGCGATTTTAG
CCTGGATAACCTGATTTTCGATGAAGGCAAACTGATTGGCTGCATTGATGTGGGCCGTG
TGGGCATTGCGGATCGTTATCAGGATCTGGCCATTCTGTGGAACTGCCTGGGCGAATTT
AGCCCGAGCCTGCAAAAACGTCTGTTTCAGAAATATGGCATTGATAATCCGGATATGAA
CAAACTGCAATTTCATCTGATGCTGGATGAATTTTTCTAATAATTAATTGAGAAGTTCC
TATACTTTCTAGAGAATAGGAACTTCGGAATAGGAACTACCGGTGCGGGTGCCAGGGCG
TGCCCCAGGGCTCCCCGGGCGCGTACTCCACGGACCAAAACGAAAAAAGGCCCCCCTTT
CGGGAGGCCTCTTTTCTGGAATTTGGTACCGAGTGCAGACGTAAAAAAAGCGGCGTGGT
TAGCCGCTTTTTTAATTGCCGGACTGCAGCAGCGATTGAGACTCAGCGAACGGAGCGGG
CGAAAGGGTTCACGATGATCAAATCTGCTCTCGACAGAATTCGGGAGGCGCA
SEQ ID NO: 65:
AGCCAGGCTGACTCCGATCGCATCAGCTATTACTCGCGCGGGTTTGAAATTGACAACTA
TCCAGATCGTTCCTCAGGTGACCTCGAGAGTTAACCAAAAAGGGGGGATTTTATCTCCC
CTTTAATTTTTCCTCGCAGATAGCAAAAAAGCGCCTTTAGGGCGCTTTTTTACATTGGT
GGCGGTGCGGGTGCCAGGGCGTGCCCCTGGGCTCCCCGGGCGCGTACTCCACGAAGTTC
CTATACTTTCTAGAGAATAGGAACTTCGGAATAGGAACTTCTAGTAGCCCGCCTAATGA
GCGGGCTTTTTTTTAATTCCCCTATTTGTTTATTTTTCTAAATACATTCAAATATGTAT
CCGCTCATGAGACAATAACCCTGATAAATGCTTCAATAATATTGAAAAAGGAAGAGTAT
GAGCATTCAGCATTTTCGTGTGGCGCTGATTCCGTTTTTTGCGGCGTTTTGCCTGCCGG
TGTTTGCGCATCCGGAAACCCTGGTGAAAGTGAAAGATGCGGAAGATCAACTGGGTGCG
CGCGTGGGCTATATTGAACTGGATCTGAACAGCGGCAAAATTCTGGAATCTTTTCGTCC
GGAAGAACGTTTTCCGATGATGAGCACCTTTAAAGTGCTGCTGTGCGGTGCGGTTCTGA
GCCGTGTGGATGCGGGCCAGGAACAACTGGGCCGTCGTATTCATTATAGCCAGAACGAT
CTGGTGGAATATAGCCCGGTGACCGAAAAACATCTGACCGATGGCATGACCGTGCGTGA
ACTGTGCAGCGCGGCGATTACCATGAGCGATAACACCGCGGCGAACCTGCTGCTGACGA
CCATTGGCGGTCCGAAAGAACTGACCGCGTTTCTGCATAACATGGGCGATCATGTGACC
CGTCTGGATCGTTGGGAACCGGAACTGAACGAAGCGATTCCGAACGATGAACGTGATAC
CACCATGCCGGCAGCAATGGCGACCACCCTGCGTAAACTGCTGACGGGTGAGCTGCTGA
CCCTGGCAAGCCGCCAGCAACTGATTGATTGGATGGAAGCGGATAAAGTGGCGGGTCCG
CTGCTGCGTAGCGCGCTGCCGGCTGGCTGGTTTATTGCGGATAAAAGCGGTGCGGGCGA
ACGTGGCAGCCGTGGCATTATTGCGGCGCTGGGCCCGGATGGTAAACCGAGCCGTATTG
TGGTGATTTATACCACCGGCAGCCAGGCGACGATGGATGAACGTAACCGTCAGATTGCG
GAAATTGGCGCGAGCCTGATTAAACATTGGTAAAGAAGTTCCTATACTTTCTAGAGAAT
AGGAACTTCGGAATAGGAACTACCGGTGCGGGTGCCAGGGCGTGCCCCCGGGCTCCCCG
GGCGCGTACTCCACGGACCAAAACGAAAAAACACCCTTTCGGGTGTCTTTTCTGGAATT
TGGTACCGAGCCTTTGGTCGAAAAAAAAAGCCCGCACTGTCAGGTGCGGGCTTTTTTCT
GTGTTTCCCTGCAGCAGCGATTGAGACTCAGCGAACGGTTGACGGTATTCCAACGTATT
TTGAGTCCCGCTGGAATCTGGGCGATGCGCTAACGGA

### REFERENCES

Petrovska L, Aspinall R J, Barber L, Clare S, Simmons C P, Stratford R, Khan S A, Lemoine N R, Frankel G, Holden D W and Dougan G. Salmonella enterica serovar Typhimurium interaction with dendritic cells: impact of the sifA gene. Cellular Microbiology, 2004; 6:1071-1084.
Hindle Z, Chatfield S N, Phillimore J, Bentley M, Johnson J, Cosgrove C A, Ghaem-Maghami M, Sexton A, Khan M, Brennan F R, Everest P, Wu T, Pickard D, Holden D W, Dougan G, Griffin G E, House D, Santangelo J D, Khan S A, Shea J E, Feldman R G, and Lewis D J. Characterization of Salmonella enterica derivatives harboring defined aroC and Salmonella pathogenicity island 2 type III secretion system (ssaV) mutations by immunization of healthy volunteers. Infect Immun. 2002; 70(7): 3457-67.
Lehouritis P, Hogan G, and Tangney M. Designer Bacteria as Intratumoural Enzyme Biofactories, Advanced Drug Delivery Reviews. 2017.
Kimura H, Zhang L, Zhao M, Hayashi K, Tsuchiya H, Tomita K, Bouvet M, Wessels J, and Hoffman R M. Targeted therapy of spinal cord glioma with a genetically modified Salmonella typhimurium. Cell Proliferation. 2010; 43:41-48.

1. A modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

2. The modified strain of claim 1, wherein said chromosomally integrated synthetic polynucleotide sequence is located within at least one genomic locus defined by any one of SEQ ID NOs: 1-30, or a sequence comprising at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs: 1-30.

3. The modified strain of claim 1 or 2, wherein the defined recombination site is an attB recombination site.

4. The modified strain of claim 2, wherein the attB recombination site is a PhiC31 attB recombination site and/or a Bxbl attB recombination site.

5. The modified strain of claim 1, wherein the chromosomally integrated synthetic polynucleotide sequence further comprises insulator regions flanking the defined recombination site.

6. The modified strain of claim 5, wherein each insulator region is selected from any one of SEQ ID NOs: 43-59, or a sequence comprising at least 70% identity to any one of SEQ ID NOs: 43-59.

7. The modified strain of claim 6, wherein each insulator region is selected from any one of SEQ ID NOs: 43-59, or a sequence comprising at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs: 43-59.

8. The modified strain of claim 1, wherein said live attenuated strain comprises two, three, four, five or six chromosomally integrated synthetic polynucleotides inserted into pre-determined genomic locations.

9. The modified strain of claim 8, wherein said live attenuated strain comprises three chromosomally integrated synthetic polynucleotides.

10. The modified strain of claim 1, wherein the chromosomally integrated synthetic polynucleotide sequence has a sequence according to SEQ ID NO: 60, 61, 62, or a sequence comprising at least 70% identity thereof.

11. The modified strain of claim 10, wherein the wherein the chromosomally integrated synthetic polynucleotide sequence has a sequence according to SEQ ID NO: 60, 61, 62, or a sequence comprising at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NO: 60, 61, 62.

12. The modified strain of claim 1, wherein the live attenuated strain is a Salmonella enterica strain.

13. The modified strain of claim 12, wherein the live attenuated strain is a Salmonella enterica serovar Typhi strain or a Salmonella enterica serovar Typhimurium strain.

14. The modified strain of claim 13, wherein the live attenuated strain is selected from the group consisting of Ty21a, CVD 908-htrA, CVD 909, Ty800, M01ZH09, x9633, x639, x9640, x8444, ZH9PA, DTY88, MD58, WT05, ZH26, SL7838, SL7207, VNP20009, A1-R, or any combinations thereof.

15. The modified strain of claim 1, wherein the live attenuated strain is a genetically engineered strain.

16. The modified strain of claim 12, wherein the live attenuated strain comprises an attenuating mutation in a Salmonella Pathogenicity Island 2 (SPI-2) gene and/or an attenuating mutation in a second gene.

17. The modified strain of claim 1, wherein the pre-determined genomic location is a genomic location of low or high transcriptional strength.

18. The modified strain of claim 1, wherein the heterologous polynucleotide sequence encodes for an immunogenic compound and/or a cancer therapeutic.

19. The modified strain of claim 1, wherein the heterologous polynucleotide has a size in the range of 1 Kbp to 10 Kbp

20. The modified strain of claim 19, wherein the heterologous polynucleotide has a size in the range of 1 Kbp to 2 Kbp.

21. A vaccine composition comprising the modified live attenuated strain of claim 1.

22. The vaccine composition of claim 21, further comprising a pharmaceutically acceptable carrier, excipient, or adjuvant.

23-30. (canceled)

31. A method of treating, inhibiting or controlling a neoplastic disease, or an infectious disease, in a subject, wherein the method comprises administering to the subject a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

32. The method of claim 31, wherein said chromosomally integrated synthetic polynucleotide sequence is located within at least one genomic locus defined by any one of SEQ ID NOs: 1-30, or a sequence comprising at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs: 1-30.

33. The method of claim 31, wherein the modified live attenuated strain is a modified live attenuated strain of Salmonella, said strain comprising at least one chromosomally integrated synthetic polynucleotide sequence inserted into a pre-determined pseudogenomic location, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

34. A method of modifying a live attenuated strain of Salmonella, said method comprising inserting a synthetic polynucleotide sequence into a pre-determined pseudogenomic location of the live attenuated strain of Salmonella, said sequence comprising at least one defined recombination site for the introduction of a heterologous polynucleotide sequence encoding a polypeptide, wherein said chromosomally integrated synthetic polynucleotide sequence is located within at least one genomic locus defined by any one of SEQ ID NOs: 1-30, or a sequence comprising at least 70% identity to any one of SEQ ID NOs: 1-30.

35. The method of claim 34, wherein said chromosomally integrated synthetic polynucleotide sequence is located within at least one genomic locus defined by any one of SEQ ID NOs: 1-30, or a sequence comprising at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs: 1-30.

36. (canceled)

37. The modified strain of claim 16, wherein the SPI-2 is a ssaV gene and the second gene is an aroC gene.

38. The method of claim 31, wherein the method is a method of treating, inhibiting or controlling a neoplastic disease.

39. The method of claim 38, wherein the neoplastic disease is a cancer.

40. The method of claim 39, wherein the cancer is selected from prostate cancer, liver cancer, renal cancer, lung cancer, breast cancer, colorectal cancer, bladder cancer, pancreatic cancer, brain cancer, hepatocellular carcinoma, lymphoma, leukaemia, gastric cancer, cervical cancer, ovarian cancer, thyroid cancer, melanoma, carcinoma, head and neck cancer, skin cancer or sarcoma.

41. The method of claim 40, wherein the cancer is selected from lung cancer, renal cancer, bladder cancer, gastric cancer, colorectal cancer, head and neck cancer or breast cancer.

42. The method of claim 31, wherein the method is a method of treating, inhibiting or controlling an infectious disease.

43. The method of claim 31, wherein the method is a method of treating, inhibiting or controlling an autoimmune disease or disorder.

44. The vaccine composition of claim 21, wherein the modified strain is formulated for oral administration, intratumoral administration, peritumoral administration, intradermal administration, subcutaneous administration, or intraperitoneal administration.

45. The method of claim 31, wherein the modified strain is to be administered in combination with an immunotherapy, chemotherapy, radiotherapy, anti-viral therapy, antibacterial therapy, antifungal therapy, or antiparasitic therapy.

46. The method of claim 45, wherein the immunotherapy is a checkpoint inhibitor, an antigen specific T cell, a therapeutic antibody, or a cancer vaccine.

47. The method of claim 31, wherein the modified strain is formulated for oral administration, intratumoral administration, peritumoral administration, intradermal administration, subcutaneous administration, or intraperitoneal administration
